(19) United States
(12) Patent Application Publication
(10) Pub. No.: US 2024/0216554 A1
(43) Pub. Date: Jul. 4, 2024

(54) RADIOIMMUNOCONJUGATES DIRECTED TO NKG2D LIGANDS FOR THE TREATMENT OF CANCER

(71) Applicant: ACTINIUM PHARMACEUTICALS, INC., New York, NY (US)

(72) Inventors: Dale L Ludwig, Rockaway, NJ (US); Eileen GEOGHEGAN, New Rochelle, NY (US); Sandesh SETH, New York, NY (US); Jesse HWANG, Forest Hills, NY (US); Paul DIAMOND, Fort Lee, NY (US)

(73) Assignee: ACTINIUM PHARMACEUTICALS, INC., New York, NY (US)

(21) Appl. No.: 18/557,988

(22) PCT Filed: May 3, 2022
(86) PCT No.: PCT/US22/27479

§ 371 Date: Oct. 30, 2023

Related U.S. Application Data
(60) Provisional application No. 63/184,017, filed on May 4, 2021.

(51) Int. Cl. A61K 51/10; A61K 45/06; C07K 16/28

(52) U.S. Cl. CPC A61K 51/1027; A61K 45/06; A61K 51/1093; C07K 16/2833; C07K 16/2851; A61K 2121/00; A61K 2123/00

(57) ABSTRACT

Provided are compositions and methods for treating a solid or hematological cancer in a subject by administering an effective amount of a radioconjugated agent that targets one or more natural killer group 2, member D (NKG2D) ligands, alone or in combination with additional therapeutic agents or modalities. The radioconjugated NKG2DL-targeting agent targets NKG2DL-positive cells, such as tumor cells, depleting those cells and bystander NKG2DL-negative cells to effect overall tumor reduction. Moreover, radiation from the radioconjugated NKG2DL-targeting agent may increase expression of the target NKG2DL, leading to a feed-forward mechanism that further enhances the accumulation of the NKG2DL-targeting agent within the tumor.

### CROSS-REFERENCE TO RELATED APPLICATIONS

This application claims priority to U.S. provisional application Ser. No. 63/184,017 filed May 4, 2021 which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on May 2, 2022, is named ATNM-020PCT_SL.txt and is 80,135 bytes in size.

### FIELD OF THE INVENTION

The present disclosure relates to the field of radiotherapeutics.

### BACKGROUND OF THE INVENTION

Surface expression of natural killer group 2, member D (NKG2D) ligands, which include MHC class I-related chains A and B (MICA and MICB) and UL16-binding proteins (ULBP1-6), are induced by stressors such as viral infection, irradiation, or malignant transformation (Liu, 2019). Despite some level of conservation among these eight ligands, each of these stress-inducible proteins is regulated uniquely and independently (Schmiedel, 2018), and some of the important transcription factors that induce NKG2D ligand expression include p53 (DNA damage and infections), heat shock factor-1 (temperature stress), and Sp1/3 (proliferative state; Schmiedel, 2018; Raulet, 2013).

As stress-inducible proteins, NKG2D ligands signal that the cell is in an abnormal state and are detected by the homodimeric NKG2D receptor expressed on the surface of immune cells (NK cells and some T cell subsets). Engagement of NKG2D ligands by the cognate NKG2D receptor causes immune effector cell activation, ultimately resulting in the killing of the abnormal cell (via perforin and granzymes) and/or cytokine release (Liu, 2019). To avoid immune surveillance and subsequent destruction, tumor cells proteolytically cleave NKG2D ligands, with the released ectodomains being detectable in the sera of cancer patients (Xing, 2020).

Unlike the highly diverse T cell receptors generated from somatic recombination where each T cell receptor recognizes a single antigen, the NKG2D receptor is germline-encoded and can recognize multiple NKG2D ligands, representing a key component of the innate immune repertoire (Zingoni, 2018). While NKG2D ligands are maintained at low levels or are absent in normal cells, many cancers, including both solid and hematopoietic cancers, are known to induce expression of these proteins (Liu, 2019; Xing, 2020; Baragano Raneros, 2015). This differential expression profile between normal and cancerous cells makes NKG2D ligands suitable for therapeutic targeting, and there are a number of strategies currently being investigated, including chimeric antigen receptor (CAR) T cells, monoclonal antibodies, and recombinant NKG2D proteins (Barber, 2007; Ding, 2018; De Andrade, 2018).

Commonly, CAR T cells recognize a specific tumor marker through an engineered scFv expressed on the cell surface (Demoulin, 2017). Thus, application across multiple cancer types is limited unless the marker is broadly distributed. However, because NKG2D is a receptor that recognizes multiple ligands present on a wide variety of tumors (e.g., bladder cancer, cervical cancer, ovarian cancer, acute myeloid leukemia, breast cancer, colorectal cancer, gastric carcinoma, hepatocellular carcinoma, melanoma, multiple myeloma, non-small-cell lung cancer, pancreatic cancer, prostate cancer, renal cell carcinoma, sarcoma; Demoulin, 2017), the extracellular ligand-binding portion of NKG2D can be exploited as part of a CAR T cell construct. Such an approach was first demonstrated in 2005, whereby NKG2D was fused to the CD3zeta chain. The resulting engineered protein displayed antitumor activity against a T cell lymphoma expressing an NKG2D ligand but was ineffective against the parental tumor lacking NKG2D ligand expression, demonstrating the specificity of the NKG2D moiety (Zhang, 2005).

Since this study, additional preclinical studies have shown that CAR T cell-based NKG2D ligand-targeting therapy confers activity against ovarian cancer, multiple myeloma, melanoma, and pancreatic cancer (Demoulin, 2017). However, in a Phase 1 study (CM-CS1, NCT02203825) conducted in eleven patients with AML/MDS or multiple myeloma, none of the patients exhibited objective tumor response, which may be related to the lack of long-term persistence of the adoptively transferred CAR T cells (Nikiforow, 2016). Despite the lack of clear clinical efficacy observed in the first-in-human trial, multiple Phase 1/2 trials are being conducted in both hematological and solid tumors: NCT03018405 (THerapeutic Immunotherapy with NKR-2 [THINK] study), NCT03466320 (LymphoDEPLEtion and THerapeutic Immunotherapy With NKR-2 [DEPLETHINK] study), NCT03370198, NCT03310008 (Standard cHemotherapy Regimen and Immunotherapy with NKR-2 [SHRINK] study), NCT03692429 (Standard cHemotherapy Regimen and Immunotherapy With Allogeneic NKG2D-based CYAD-101 Chimeric Antigen Receptor T-cells [alloSHRINK]).

Clinical efficacy of these CAR T cells has yet to be demonstrated, but no dose-limiting toxicities were observed in the Phase 1 CM-CS1 trial (Nikiforow, 2016), suggesting that therapeutic reconfiguration of the NKG2D moiety may be safe but requires further modification or optimization to elicit durable response (e.g., progression-free survival, overall survival).

Accordingly, an object of the present disclosure is to provide novel agents that target NKG2D ligands, such as those expressed on solid and hematopoietic cancers, and improved therapeutic methods for the treatment of patients with such cancers.

### SUMMARY OF THE INVENTION

The present disclosure provides therapeutic and diagnostic methods for the treatment of cancer or precancerous proliferative disorders by targeting NKG2D ligands (NKG2DLs), for example through administration of compositions including a radioconjugated NKG2DL targeting agent. The radioconjugated NKG2DL targeting agents may, for example, include radioconjugated NKG2D receptor proteins, radioconjugated antibodies that recognize NKG2DLs, or radioconjugated small domain proteins such as a DARPin, anticalin, or affimer, or a peptide, aptamer, or small molecule that binds to an NKG2DL and thus delivers DNA-damage inducing radiation to cells expressing NKG2DLs.

The radioconjugated NKG2DL targeting agents useful for therapeutic interventions may include a radioisotope (radionuclide) such as ¹³¹I, ¹²⁵I, ¹²³I, ⁹⁰Y, ¹⁷⁷Lu, ¹⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, or ²¹²Pb, or any combination thereof. In one variation, the radioconjugated NKG2DL targeting agents may include ¹³¹I, ⁹⁰Y, ¹⁷⁷Lu, ²²⁵Ac, ²¹³Bi, ²¹¹At, ²²⁷Th, or ²¹²Pb, or any combination thereof.

Therapeutic methods of the present disclosure generally include administering to a patient an effective amount of the radioconjugated NKG2DL targeting agent. The effective amount may, for example, be a maximum tolerated dose (MTD) or a fractioned dose wherein the total amount of radiation administered in the fractioned doses is the MTD.

A composition including or quantity/amount of the radioconjugated NKG2DL targeting agent may, for example, include a radiolabeled fraction and a non-radiolabeled fraction of the NKG2DL targeting agent. As such, an effective amount of the radioconjugated NKG2DL targeting agent may include a total protein dose of less than 100 mg, such as from 1 mg to 60 mg, or 5 mg to 45 mg. The total protein dose may be from 0.001 mg/kg to 3 mg/kg body weight of the subject, such as from 0.005 mg/kg to 2 mg/kg body weight of the subject. The total protein dose may be less than 2 mg/kg, or less than 1 mg/kg, less than 0.5 mg/kg, or even less than 0.1 mg/kg.

An effective amount of a radioconjugated NKG2DL targeting agent, such as an ²²⁵Ac-NKG2D receptor, or ²²⁵Ac-anti-NKG2DL antibody, peptide, or small molecule, may, for example, include a radiation dose of 0.1 to 50 μCi/kg body weight of the subject, such as 0.1 to 5 μCi/kg body weight of the subject, or 5 to 20 μCi/kg subject body weight, or a radiation dose of 2 μCi to 2 mCi, or 2 μCi to 250 μCi, or 75 μCi to 400 μCi in a non-weight-based radiation dose.

An effective amount of a radioconjugated NKG2DL targeting agent, such as an ¹⁷⁷Lu-NKG2D receptor, or ¹⁷⁷Lu-anti-NKG2DL binding antibody, peptide, or small molecule, may, for example, include a radiation dose of 1 to 1000 μCi/kg body weight of the subject, such as 5 to 250 μCi/kg body weight of the subject, or 50 to 450 μCi/kg body weight, or a radiation dose of 10 mCi to 30 mCi, or 100 μCi to 3 mCi, or 3 mCi to 30 mCi in anon-weight-based radiation dose.

An effective amount of a radioconjugated NKG2DL targeting agent, such as an ¹³¹I-NKG2D receptor, or ¹³¹I-anti-NKG2DL antibody, peptide, or small molecule, may, for example, include a dose of below 1200 mCi in a non-weight-based radiation dose, such as from at least 1 mCi to below 100 mCi, or at least 10 mCi to below 200 mCi.

The effective amount of the radioconjugated NKG2DL targeting agent, may, for example, depend on the configuration of the targeting agent, i.e., full length protein or antibody, antibody fragment, minibody, nanobody, etc. For example, when the NKG2DL targeting agent includes an ²²⁵Ac-anti-NKG2DL targeting agent that is a full-length antibody, the dose may, for example, be below 5 μCi/kg body weight of the subject, such as 0.1 to 5 μCi/kg body weight of the subject. Alternatively, when the NKG2DL targeting agent includes an ²²⁵Ac-anti-NKG2DL targeting agent that is an antibody fragment such as a Fab or Fab₂ fragment, small domain protein such as a DARPin, anticalin, affimer, or aptamer, or small molecule, the dose may, for example, be greater than 5 μCi/kg body weight of the subject, such as 5 to 20 μCi/kg body weight of the subject.

The radioconjugated NKG2DL targeting agent may, for example, be administered according to a dosing schedule selected from the group consisting of one dose every 5, 7, 10, 12, 14, 20, 24, 28, 35, and 42 days throughout a treatment period, wherein the treatment period includes at least two doses.

The radioconjugated NKG2DL targeting agent may, for example, be administered according to a dose schedule that includes 2 doses, such as on days 1 and 5, 6, 7, 8, 9, or 10 of a treatment period, or days 1 and 8 of a treatment period.

The radioconjugated NKG2DL targeting agent may, for example, be administered as a single bolus or infusion.

Each administration of the radioconjugated NKG2DL targeting agent may, for example, be as a subject specific dose (a patient-specific dose), wherein each of a protein dose and a radiation dose are selected based on subject specific characteristics (e.g., weight, age, gender, health status, nature and severity of the cancer or tumor, etc.).

The methods may, for example, further include administration of one or more further therapeutic agents, such as a chemotherapeutic agent, an anti-inflammatory agent, an immunosuppressive agent, an immunomodulatory agent, an antimyeloma agent, a cytokine, an HDAC inhibitor, an LSD1 inhibitor, or any combination thereof. Exemplary chemotherapeutic agents include at least radiosensitizers that may synergize with the radioconjugated NKG2DL targeting agent, such as temozolomide, cisplatin, and/or fluorouracil.

The methods may, for example, further include administration of one or more immune checkpoint therapies. Exemplary immune checkpoint therapies include an antagonist antibody against CTLA-4, PD-1, TIM3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, A2aR, or CGEN-15049, or any combination thereof. The immune checkpoint therapy may, for example, include an antibody against an immune checkpoint protein selected from the group consisting of one or more antibodies against PD-1, PD-L1, CTLA-4, TIM3, LAG3, or VISTA, and any combination thereof. The immune checkpoint therapy may, for example, be provided in a subject effective amount including a dose of 0.1 mg/kg to 50 mg/kg of the subject/patient's body weight, such as 0.1-5 mg/kg, or 5-30 mg/kg.

The methods may, for example, further include administration of one or more DNA damage response inhibitors (DDRi). An exemplary DDRi includes at least one or more antibodies or small molecules targeting poly(ADP-ribose) polymerase (i.e., a poly(ADP-ribose) polymerase inhibitor or PARPi). The PARPi may be a small molecule therapeutic selected from the group consisting of olaparib, niraparib, rucaparib, talazoparib, and a combination thereof. The PARPi may, for example, be provided in a subject effective amount including 0.1 mg/day-1200 mg/day, such as 0.100 mg/day-600 mg/day, or 0.25 mg/day-1 mg/day. Exemplary subject effective amounts include 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, and 1000 mg, taken orally in one or two doses per day. Another exemplary DDRi includes an inhibitor of Ataxia telangiectasia mutated (ATM), Ataxia talangiectasia mutated and Rad-3 related (ATR), or Wee1. Exemplary inhibitors of ATM include KU-55933, KU-59403, wortmannin, CP466722, and KU-60019. Exemplary inhibitors of ATR include at least Schisandrin B, NU6027, NVP-BEA235, VE-821, VE-822, AZ20, and AZD6738. Exemplary inhibitors of Wee1 include AZD-1775 (i.e., adavosertib).

The methods may, for example, further include administration of one or more CD47 blockades. The CD47 blockade may, for example, include a monoclonal antibody against either of CD47 and SIRPα which blocks binding of CD47 to SIRPα or a SIRPα-Fc fusion protein that blocks CD47 binding to endogenous SIRPα. The CD47 blockade may, for example, include magrolimab, lemzoparlimab, AO-176, AK117, IMC-002, IBI-188, IBI-322, BI 766063, ZL-1201, ALX148, RRx-001, ES004, SRF231, SHR-1603, TJC4, TTI-621, or TTI-622, or any combination thereof. Exemplary effective doses for the CD47 blockade include 0.05 to 5 mg/kg patient weight. The CD47 blockade may, for example, include agents that modulate the expression of CD47 and/or SIRPα, such as a nucleic acid approach, for example, an antisense agent. An exemplary agent includes phosphorodiamidate morpholino oligomers (PMO) that block translation of CD47, such as MBT-001 (Morphiex).

The methods may, for example, further include administration of a therapeutic modality such as radiation therapy. Exemplary radiation therapies include external beam radiation and brachytherapy.

The methods may, for example, further include administration of any combination of the further therapeutic agents or modalities. Exemplary combinations include at least one or more DDRi, one or more immune checkpoint therapies, one or more CD47 blockades, one or more chemotherapeutics, one or more radiation therapies (e.g., external beam radiation or brachytherapy), or any combination thereof.

The radioconjugated NKG2DL targeting agent and the one or more further therapeutic agents may, for example, be administered simultaneously or sequentially. When more than one additional therapeutic agent is administered, the agents may, for example, be administered simultaneously or sequentially.

The radioconjugated NKG2DL targeting agent may, for example, be a portion of a multispecific antibody. Thus, the methods may include administering to the subject an effective amount of a multispecific antibody, wherein the multispecific antibody includes: a first target recognition component that specifically binds to a NKG2DL, and a second target recognition component that binds to a different epitope of the same NKG2DL or a different NKG2DL than the first target recognition component. The NKG2DL targeting agent may, for example, be a multispecific antibody against an NKG2DL and a second antigen that is not a NKG2DL such as a different cancer-associated antigen.

Additional features, advantages, and embodiments of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

The present disclosure provides compositions and methods for treating cancer or precancerous proliferative disorders by targeting cytotoxic radiation to one or more NKG2DLs on cancer cells and/or precancerous cells. Prior therapies envisioned to target such ligands have included cell-based therapies, such as CAR-T cell-based NKG2DL-targeting therapies. An alternative to such cell-based therapies is the use of biologics to target tumor-specific surface proteins. Within the context of the NKG2D-NKG2DL axis, this approach may incorporate either a soluble NKG2D receptor or an agent (e.g., monoclonal antibody, peptide, synthetically designed protein, small molecule) that recognizes NKG2DLs. A soluble NKG2D receptor by itself may not have any cytolytic activity even after binding to a cancer cell, but fusion to the IgG1 Fc domain would permit ADCC- or CDC-mediated tumor cell lysis. By initiating an innate immune response against the tumor, NKG2D-Fc or antibodies against NKG2DLs may confer clinical benefit even among patients for whom T cell-based therapy has become ineffective due to immunosuppressive signaling events e.g., engagement of the PD-L1/PD-1 or CTLA-4 pathways (Quezada, 2013), wherein immune checkpoint inhibitors can re-activate T cells, but a durable response may still not be observed in patients (Wang, 2019; Yoon, 2018).

While ADCC and CDC may potently ablate cancerous cells, these cytotoxic mechanisms may be ineffective against tumor cells that lack sufficient levels of cell surface NKG2DLs (e.g., due to proteolytic cleavage; Xing, 2020). The presently disclosed invention overcomes this shortfall and enhances the cell-killing capability of these tumor-targeting agents through activation of cell death pathways to which cancer cells cannot easily develop resistance.

A strategy of the presently disclosed invention includes radioconjugation (radiolabeling) of NKG2DL-targeting agents to deliver radiation specifically to tumor cells that bear NKG2DLs on the cell surface. Multiple types of targeting agents, such as a soluble recombinant NKG2D receptor or NKG2DL-binding proteins, e.g., antibodies, peptides, or small molecules, can be labeled with radionuclides to deliver DNA damaging radiation and subsequent tumor destruction. These radionuclides include, but are not limited to, Actinium-225, Astatine-211, Bismuth-213, Iodine-131, Lead-212, Lutetium-177, Radium-223, Thorium-227, Yttrium-90. Of these, Actinium-225 (²²⁵Ac) displays characteristics that render this radionuclide particularly suitable for anticancer therapy.

²²⁵Ac emits four high linear energy transfer alpha particles during its decay profile over a very short distance of about 3-4 cells' thickness (Pouget, 2011), making this payload very potent in effecting lethal double-strand DNA (dsDNA) breaks by direct ionizing radiation. This short path length also makes ²²⁵Ac much safer to handle compared to beta-emitting isotopes that have longer ranges (Nelson, 2020). By conjugating a radioactive payload to the NKG2DL-targeting agent, such as via a stable metal chelator, radiation can be delivered specifically and systemically to both primary tumors and metastatic tumors, which often remain undetected and are not amenable to treatment by external beam radiation, while minimizing exposure of healthy tissues that do not express (or do not overexpress) NKG2DLs. Moreover, epithelial-mesenchymal transition (EMT), which is characterized by the loss of cell-cell adhesion and contributes to metastatic potential, has been shown to be associated with higher expression of NKG2DLs (López-Soto, 2017, 2013). Thus, a targeted radiation approach using NKG2DL-binding moieties may be especially suitable to ablate these aggressive cells before metastatic tumors may be established in distant niches.

Radioconjugation has several advantages over drug conjugation approaches. Unlike antibody-drug conjugates, radioconjugates do not require internalization because, for example, alpha particles can cross multiple cellular membranes to reach the nuclei, causing clusters of dsDNA breaks that cannot be easily repaired (Nelson, 2020). Furthermore, whereas antibody-drug conjugates require high surface density of the targeted molecule to deliver sufficient quantities of the toxic payload (Sadekar, 2015), radioligands are less sensitive to target expression level since a single alpha particle is capable of inducing cancer cell death (Neti, 2006). “Cross-firing” is another advantage of radioconjugates, whereby radiation is delivered to both the targeted cancer cells and to adjacent malignant cells (Haberkorn, 2017). In this way, radioimmunotherapy can exert clinical/therapeutic efficacy even if the target expression profile is heterogeneous within the tumor.

Lastly, targeting the NKG2D-NKG2DL axis using radioimmunotherapy makes clinical sense for another important reason: the potential to activate a feed-forward mechanism of tumor destruction. Ionizing radiation is known to upregulate NKG2DL levels on the surface of multiple cell lines derived from cancers (Weiss, 2018; Son, 2014; Heo, 2015; Lee, 2018), and in the case of the glioma cell line LN-18, radiation-induced upregulation of the NKG2DLs was shown to depend on the ATM signaling pathway (Weiss, 2018). Importantly, NKG2DL upregulation post-irradiation was recapitulated in vivo in mice with orthotopic glioma, and by using near-infrared fluorescent protein-expressing glioma cells, detection of NKG2DLs could be pinpointed specifically to cancer cells, excluding contaminating signals from normal cells. Since external radiation is documented to induce NKG2DLs on malignant cells, internal administration of a targeted radioconjugated agent (e.g., ²²⁵Ac-NKG2D-Fc, ²²⁵Ac-NKG2DL-specific antibody) would lead to similar induction of NKG2DLs within tumors, causing further accumulation of the radioconjugated therapeutic agent within the malignant tissues. Thus, by eliciting a radiation stress response, the presently disclosed targeted radiation approach induces the therapeutic target itself, leading to a feed-forward mechanism of tumor eradication.

Accordingly, the presently disclosed agents and methods for deposition of a radioconjugated NKG2DL targeting agent to tumor cells expressing one or more NKG2DLs, such as by administration of an ²²⁵Ac-labeled agent would safely and efficaciously ablate tumors and prolong the survival of cancer patients. Moreover, lower amounts of total antibody and/or targeting agent may be needed and used to achieve clinical efficacy with the radioconjugate approach.

As such, the present disclosure provides novel compositions and methods for treating cancer by targeting cells expressing NKG2DLs. Specifically, the present disclosure relates to NKG2DL targeting agents that are radioconjugated and their use in treating cancers or precancerous proliferative disorders in mammalian subjects, such as human patients, in need of treatment therefor, either alone or in combination or conjunction with other therapeutic agents and/or modalities. Related methods for treating a cancer or precancerous proliferative disorder are also provided which generally include administering to the subject/patient an effective amount of a radioconjugated NKG2DL targeting agent, such as a radiolabeled anti-NKG2DL antibody, soluble NKG2D receptor, NKG2DL-binding peptide, or NKG2DL-binding small molecule, alone or in combination with one or more additional therapeutic agents or modalities.

The additional therapeutic agents or modalities may, for example, include any one or more immune checkpoint therapies, one or more inhibitors of a component of the DNA damage response pathway (i.e., a DNA damage response inhibitor, DDRi, such as one or more agents against poly(ADP-ribose) polymerase, i.e., PARPi), one or more CD47/SIRPα axis blockades, one or more HDAC inhibitors, one or more LSD1 inhibitors, one or more small molecule anti-cancer agents, one or more chemotherapeutic agents such as radiosensitizers, of these agents, one or more anti-inflammatory agents, one or more immunosuppressive agents, one or more immunomodulatory agents, one or more antimyeloma agents, one or more cytokines, external beam radiation, brachytherapy, adoptive cell therapy or any combination thereof.

The present disclosure further provides methods for diagnosing patients having NKG2DL-positive cancer, such as NKG2DL-positive hematological (liquid) cancers or NKG2DL-positive solid tumor cancers, followed by treating those patients according to any of the methods disclosed herein.

Prior to setting forth the invention in greater detail, it may be helpful to an understanding thereof to set forth definitions of certain terms to be used hereinafter.

### Definitions and Abbreviations

The singular forms “a,” “an,” “the” and the like include plural referents unless the context clearly dictates otherwise. Thus, for example, reference to “an” antibody includes both a single antibody and a plurality of different antibodies.

The term “about” when used before a numerical designation, e.g., temperature, time, amount, and concentration, including a range, indicates approximations which may vary by ±10%, ±5%, or ±1%.

As used herein, “administer”, with respect to a targeting agent such as an antibody, antibody fragment, Fab fragment, or aptamer, means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in International Pub. No. WO 2016/187514 or U.S. Pat. No. 10,736,975, each incorporated by reference herein.

In addition, in this disclosure, antibodies may be formulated using one or more routinely used pharmaceutically acceptable carriers or excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, scFv, diabodies, minibodies, nanobodies, sdAb; (d) naturally occurring and non-naturally occurring, such as wholly synthetic antibodies, IgG-Fc-silent, and chimeric; and (e) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies used may be human, humanized or nonhuman. When a specific aspect of the present disclosure refers to or recites an “antibody,” it is envisioned as referring to any of the full-length antibodies or fragments thereof disclosed herein, unless explicitly denoted otherwise. Any such types of antibodies may, for example, be used in or embodied in the various aspects of the invention.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

A “complementarity-determining region”, or “CDR”, refers to amino acid sequences that, together, define the binding affinity and specificity of the variable region of a native immunoglobulin binding site. There are three CDRs in each of the light and heavy chains of an antibody. CDRs, framework regions and variable regions in an antibody chain may, for example, be delineated/defined according to the Kabat or IMGT numbering systems.

A “framework region”, or “FR”, refers to amino acid sequences interposed between CDRs, typically conserved, that act as the scaffold between the CDRs.

A “constant region” refers to the portion of an antibody molecule that is consistent for a class of antibodies and is defined by the type of light and heavy chains. For example, a light chain constant region can be of the kappa or lambda chain type and a heavy chain constant region can be of one of the five chain isotypes: alpha, delta, epsilon, gamma or mu. This constant region, in general, can confer effector functions exhibited by the antibodies. Heavy chains of various subclasses (such as the IgG subclass of heavy chains) are mainly responsible for different effector functions.

As used herein, a “NKG2DL targeting agent” may, for example, be an antibody as defined herein, e.g., full length antibody, monoclonal antibody, NKG2DL-binding antibody fragment, minibody, nanobody, etc., that binds to any NKG2DL with a high immunoreactivity. A NKG2DL targeting agent may, for example, include or be a soluble recombinant NKG2D receptor, e.g., including or consisting of the extracellular domain of NKG2D protein fused (directly or via a linker amino acid sequence) to the Fc domain of an antibody such as an IgG1 (which Fc may, for example, be ablated for immune effector function or enhanced for immune effector function), a small domain protein such as a DARPin, anticalin, or affimer, or a peptide, aptamer, or small molecule that binds to one or more NKG2DLs.

As used herein, the term “DARPin” refers to a designed ankyrin repeat protein (a type of antibody mimetic protein) having high selectivity and high affinity for a specific protein. DARPins have a molecular weight of 14 to 21 kDa, consist of 2 to 5 ankyrin repeat motifs. They include a core region having a conserved amino acid sequence that provides structure and a variable target binding region that resides outside of the core and binds to a target. DARPins may, for example, further include an immune cell modulation motif, such as any described hereinabove.

As used herein, the term “Anticalin” refers to a scaffold protein that is a single-chain-based antibody mimetic capable of specifically binding to an antigen and has a size of about 20 kDa. Anticalin molecules are generated by combinatorial design from natural lipocalins, which are abundant plasma proteins in humans, and reveal a simple, compact fold dominated by a central β-barrel, supporting four structurally variable loops that form a binding site.

As used herein, an “Affimer” is a small, highly stable protein engineered to display peptide loops which provide a high affinity binding surface for a specific target protein. It is generally a protein of low molecular weight, 12-14 kDa, derived from the cysteine protease inhibitor family of cystatins. Affimer proteins are composed of a scaffold, which is a stable protein based on the cystatin protein fold. They display two peptide loops and an N-terminal sequence that can be randomized to bind different target proteins with high affinity and specificity similar to antibodies. Stabilization of the peptide upon the protein scaffold constrains the possible conformations which the peptide may take, thus increasing the binding affinity and specificity compared to libraries of free peptides.

As used herein, an “Aptamer” is a single stranded oligonucleotide that can naturally fold into a 3-dimensional structure and bind specifically to biosurfaces, a target compound or a moiety. These small nucleic acid molecules are essentially a chemical equivalent of antibodies. Aptamers are highly specific, relatively small in size, and non-immunogenic. Aptamers are generally selected from a biopanning method known as SELEX (Systematic Evolution of Ligands by Exponential enrichment). The SELEX process is a method for the in vitro evolution of nucleic acid molecules with highly specific binding to target molecules and is described in, e.g., U.S. Pat. No. 5,270,163 (see also Int'l Pub. No. WO 91/19813) entitled “Nucleic Acid Ligands”. Each SELEX-identified nucleic acid ligand is a specific ligand of a given target compound or molecule. Methods of generating an aptamer for any given target are well known in the art.

As used herein, “Immunoreactivity” refers to a measure of the ability of an immunoglobulin to recognize and bind to a specific antigen. “Specific binding” or “specifically binds” or “binds” refers to the targeting agent's ability to bind to an antigen or an epitope within the antigen with greater affinity than other epitopes or antigens within a relevant milieu such as within a patient's body. Typically, the targeting agent binds to the antigen or the epitope within the antigen with an equilibrium dissociation constant (K_{D}) of about 1×10⁻⁷ M or less, for example about 1×10⁻⁸ M or less, about 1×10⁻⁹ M or less, about 1×10⁻¹⁰ M or less, about 1×10⁻¹¹ M or less, or about 1×10⁻¹²M or less, typically with the K_{D} that is at least one hundred fold less than its K_{D} for binding to a nonspecific antigen (e.g., BSA, casein). The dissociation constant may be measured using standard procedures. For example, a targeting agent specifically bound to a target is not displaced by a nonsimilar competitor provided in similar concentration amounts, or even when provided at 10× or 100× excess. Alternatively, a targeting agent may be considered to specifically bind to an antigen when it preferentially recognizes its target antigen in a complex mixture of proteins and/or macromolecules such as within a subject. Targeting agents that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

An “epitope” refers to the target molecule site (e.g., at least a portion of an antigen) that is capable of being recognized by, and bound by, a targeting agent such as an antibody, antibody fragment, Fab fragment, or aptamer. For a protein antigen, for example, this may refer to the region of the protein (i.e., amino acids, and particularly their side chains) that is bound by the antibody. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

As used herein, the terms “proliferative disorder” and “cancer” may be used interchangeably and may include, without limitation, a solid cancer (e.g., a tumor) or a hematological cancer. “Solid cancers” that may be treated according the invention include, without limitation, carcinoma, sarcoma, bone cancer, osteosarcoma, Ewing's sarcoma, pancreatic cancer, skin cancer, cancer of the head or neck, cutaneous or intraocular malignant melanoma, uterine cancer, ovarian cancer, prostate cancer, rectal cancer, colon cancer, cancer of the anal region, stomach cancer, GIST, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, mesothelioma, environmentally-induced cancers including those induced by asbestos.

The solid cancer treated according to the invention may, for example, be breast cancer, endocrine-therapy resistant breast cancer, tamoxifen-resistant breast cancer, triple negative breast cancer (TNBC), HER3-positive breast cancer, HER2-positive breast cancer, gastric cancer, bladder cancer, cervical cancer, endometrial cancer, skin cancer such as melanoma, stomach cancer, testicular cancer, esophageal cancer, bronchioloalveolar cancer, prostate cancer, castration-resistant prostate cancer (CRPC), endocrine resistant prostate cancer, colorectal cancer, urothelial cancer, ovarian cancer, cervical epidermoid cancer, pancreatic cancer, lung cancer such as non-small cell lung carcinoma (NSCLC) or small cell lung cancer (SCLC), renal cancer, liver cancer, hepatocellular carcinoma (HCC), cholangiocarcinoma, adrenal gland carcinoma, head and neck cancer such as head and neck squamous cell cancer/carcinoma, or any combination thereof. Precancerous proliferative disorders that may be treated according to the invention include, for example, Barrett's esophagus.

Hematological cancers/malignancies that may be treated according to the various aspects of the invention include, for example, leukemia, acute leukemia, acute myeloid leukemia (AML), acute promyelocytic leukemia, chronic myelogenous leukemia (CML; a/k/a chronic myeloid leukemia), hairy cell leukemia, myelodysplastic syndrome (MDS), myeloproliferative neoplasms (MPNs), acute lymphoblastic leukemia (ALL), chronic lymphocytic leukemia (CLL), lymphoma, Hodgkin lymphoma, non-Hodgkin lymphoma, mantle cell lymphoma, diffuse large B-cell lymphoma (DLBCL), cutaneous T-cell lymphoma (CTCL), peripheral T-cell lymphoma (CTCL), Burkitt lymphoma, follicular lymphoma, high-grade B-cell lymphoma, Waldenstrom's macroglobulinaemia (WM), and multiple myeloma.

A solid cancer treated by any of the aspects or embodiments of the invention may be non-metastatic or metastatic, such as a non-metastatic form or a metastatic form of any of the solid tumor cancers disclosed herein. The cancers treated by any of the aspects or embodiments of the invention, whether a solid cancer or hematological cancer, may, for example, be relapsed and/or refractory (“r/r”).

A NKG2DL targeting agent is be labeled with a radioisotope for use in or embodiment in the various aspects of the invention. As used herein, “radioisotope” is synonymous with “radionuclide” and can be an alpha-emitting isotope, a beta-emitting isotope, and/or a gamma-emitting isotope. Examples of radioisotopes for therapeutic use include the following: ¹³¹I, ¹²⁵I, ¹²³I, ⁹⁰Y, ¹⁷⁷Lu ¹⁸⁶Re, ¹⁸⁸Re, ⁸⁹Sr, ¹⁵³Sm ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb and ¹⁰³Pd. In specific examples, the NKG2DL targeting agent may be radiolabeled with ²²⁵Ac, a high energy alpha particle emitting radionuclide with a 10-day half-life and short path length (<100 μm), or with ²²⁷Th another alpha particle emitter, or with ¹⁷⁷Lu, a beta particle emitter.

Methods for affixing a radioisotope to a protein such as an antibody or antibody fragment (i.e., “labeling” the protein such as antibody with a radioisotope) are well known. Suitable methods for radiolabeling are described, for example, in U.S. Pat. Nos. 10,420,851, 9,603,954, Int'l Pub. No. WO 2017155937 and U.S. Provisional Patent Application No. 63/119,093, filed Nov. 30, 2020 and titled “Compositions and methods for preparation of site-specific radioconjugates,” all of which are incorporated by reference herein.

The radiolabeled NKG2DL targeting agent may, for example, include the radioisotope ²²⁵Ac (“²²⁵Ac-labeled” or ²²⁵Ac-conjugated NKG2DL targeting agent), and the effective amount may, for example, be at or below 50.0 μCi/kg (i.e., where the amount of ²²⁵Ac administered to the subject delivers a radiation dose of below 50.0 μCi per kilogram of subject's body weight). When the NKG2DL targeting agent is ²²⁵Ac-labeled, the effective amount may, for example, be at or below 50 μCi/kg, 40 μCi/kg, 30 μCi/kg, 20 μCi/kg, 10 μCi/kg, 5 μCi/kg, 4 μCi/kg, 3 μCi/kg, 2 μCi/kg, 1 μCi/kg, or even 0.5 μCi/kg. When the NKG2DL targeting agent is ²²⁵Ac-labeled, the effective amount may, for example, be at least 0.05 μCi/kg, or 0.1 μCi/kg, 0.2 μCi/kg, 0.3 μCi/kg, 0.4 μCi/kg, 0.5 μCi/kg, 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, 10 μCi/kg, 12 μCi/kg, 14 μCi/kg, 15 μCi/kg, 16 μCi/kg, 18 μCi/kg, 20 μCi/kg, 30 μCi/kg, or 40 μCi/kg. The ²²⁵Ac-labeled NKG2DL targeting agent may, for example, be administered at a dose that includes any combination of any of upper and lower limits as described herein, such as from at least 0.1 μCi/kg to at or below 5 μCi/kg, or from at least 5 μCi/kg to at or below 20 μCi/kg.

The NKG2DL targeting agent may, for example, be ²²⁵Ac-labeled, and the effective amount may, for example, be below 2 mCi (i.e., wherein the ²²⁵Ac is administered to the subject in a non-weight-based dosage). The effective dose of the ²²⁵Ac-labeled NKG2DL targeting agent may, for example, be at or below 1 mCi, such as 0.9 mCi, 0.8 mCi, 0.7 mCi, 0.6 mCi, 0.5 mCi, 0.4 mCi, 0.3 mCi, 0.2 mCi, 0.1 mCi, 90 μCi, 80 μCi, 70 μCi, 60 μCi, 50 μCi, 40 μCi, 30 μCi, 20 μCi, 10 μCi, or 5 μCi. The effective amount of ²²⁵Ac-labeled NKG2DL targeting agent may, for example, be at least 2 μCi, such as at least 5 μCi, 10 μCi, 20 μCi, 30 μCi, 40 μCi, 50 μCi, 60 μCi, 70 μCi, 80 μCi, 90 μCi, 100 μCi, 200 μCi, 300 μCi, 400 μCi, 500 μCi, 600 μCi, 700 μCi, 800 μCi, 900 μCi, 1 mCi, 1.1 mCi, 1.2 mCi, 1.3 mCi, 1.4 mCi, or 1.5 mCi. The ²²⁵Ac-labeled NKG2DL targeting agent may, for example, be administered at a dose that includes any combination of upper and lower limits as described herein, such as from at least 2 μCi to at or below 1 mCi, or from at least 2 μCi to at or below 250 μCi, or from 75 μCi to at or below 400 μCi.

The ²²⁵Ac-labeled NKG2DL targeting agent may, for example, include a single dose that delivers less than 12 Gy, or less than 8 Gy, or less than 6 Gy, or less than 4 Gy, or less than 2 Gy, such as doses of 2 Gy to 8 Gy, to the subject, such as predominantly to the targeted solid tumor.

The radiolabeled NKG2DL targeting agent may, for example, include the radioisotope ¹⁷⁷Lu (“¹⁷⁷Lu-labeled”), and the effective amount may, for example, be at or below 1 mCi/kg (i.e., where the amount of ¹⁷⁷Lu-labeled NKG2DL targeting agent administered to the subject delivers a radiation dose of below 1000 mCi per kilogram of subject's body weight). When the NKG2DL targeting agent is ¹⁷⁷Lu-labeled, the effective amount may, for example, be at or below 900 μCi/kg, at or below 800 μCi/kg, at or below 700 μCi/kg, at or below 600 μCi/kg, at or below 500 μCi/kg, at or below 400 μCi/kg, at or below 300 μCi/kg, at or below 200 μCi/kg, at or below 150 μCi/kg, at or below 100 μCi/kg, at or below 80 μCi/kg, at or below 60 μCi/kg, at or below 50 μCi/kg, at or below 40 μCi/kg, at or below 30 μCi/kg, at or below 20 μCi/kg, at or below 10 μCi/kg, at or below 5 μCi/kg, or at or below 1 μCi/kg. The effective amount of the ¹⁷⁷Lu-labeled NKG2DL targeting agent may, for example, be at least 1 μCi/kg, 2.5 μCi/kg, 5 μCi/kg, 10 μCi/kg, 20 μCi/kg, 30 μCi/kg, 40 μCi/kg, 50 μCi/kg, 60 μCi/kg, 70 μCi/kg, 80 μCi/kg, 90 μCi/kg, 100 μCi/kg, 150 μCi/kg, 200 μCi/kg, 250 μCi/kg, 300 μCi/kg, 350 μCi/kg, 400 μCi/kg or 450 μCi/kg. An ¹⁷⁷Lu-labeled NKG2DL targeting agent may, for example, be administered at a dose that includes any combination of upper and lower limits as described herein, such as from at least 5 mCi/kg to at or below 50 μCi/kg, or from at least 50 mCi/kg to at or below 500 μCi/kg.

The NKG2DL targeting agent may ¹⁷⁷Lu-labeled, and the effective amount may, for example, be at or below 45 mCi, such as at or below 40 mCi, 30 mCi, 20 mCi, 10 mCi, 5 mCi, 3.0 mCi, 2.0 mCi, 1.0 mCi, 800 μCi, 600 μCi, 400 μCi, 200 μCi, 100 μCi, or 50 μCi. The effective amount of ¹⁷⁷Lu-labeled NKG2DL targeting agent may, for example, be at least 10 μCi, such as at least 25 μCi, 50 μCi, 100 μCi, 200 μCi, 300 μCi, 400 μCi, 500 μCi, 600 μCi, 700 μCi, 800 μCi, 900 μCi, 1 mCi, 2 mCi, 3 mCi, 4 mCi, 5 mCi, 10 mCi, 15 mCi, 20 mCi, 25 mCi, 30 mCi. An ¹⁷⁷Lu-labeled NKG2DL targeting agent may, for example, be administered at a dose that includes any combination of upper and lower limits as described herein, such as from at least 10 mCi to at or below 30 mCi, or from at least 100 μCi to at or below 3 mCi, or from 3 mCi to at or below 30 mCi.

The radiolabeled NKG2DL targeting agent may include the radioisotope ¹³¹I (“¹³¹I-labeled”), and the effective amount may, for example, be at or below 1200 mCi (i.e., where the amount of ¹³¹I administered to the subject delivers a total body radiation dose of below 1200 mCi in a non-weight-based dose). The effective amount of the ¹³¹I-labeled NKG2DL targeting agent may, for example, be at or below 1100 mCi, at or below 1000 mCi, at or below 900 mCi, at or below 800 mCi, at or below 700 mCi, at or below 600 mCi, at or below 500 mCi, at or below 400 mCi, at or below 300 mCi, at or below 200 mCi, at or below 150 mCi, or at or below 100 mCi. The effective amount of the ¹³¹I-labeled NKG2DL targeting agent may, for example, be at or below 200 mCi, such as at or below 190 mCi, 180 mCi, 170 mCi, 160 mCi, 150 mCi, 140 mCi, 130 mCi, 120 mCi, 110 mCi, 100 mCi, 90 mCi, 80 mCi, 70 mCi, 60 mCi, or 50 mCi. The effective amount of the ¹³¹I-labeled NKG2DL targeting agent may, for example, be at least 1 mCi, such as at least 2 mCi, 3 mCi, 4 mCi, 5 mCi, 6 mCi, 7 mCi, 8 mCi, 9 mCi, 10 mCi, 20 mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, 160 mCi, 170 mCi, 180 mCi, 190 mCi, 200 mCi, 250 mCi, 300 mCi, 350 mCi, 400 mCi, 450 mCi, 500 mCi. An ¹³¹I-labeled NKG2DL targeting agent may, for example, be administered at a dose that includes any combination of upper and lower limits as described herein, such as from at least 1 mCi to at or below 100 mCi, or at least 10 mCi to at or below 200 mCi.

While select radionuclides have been disclosed in detail herein, any from the list provided above or known in the art to be suitable for therapeutic use may be used to radiolabel a NKG2DL targeting agent for use in various the aspects of the present disclosure.

As used herein, a composition including a radiolabeled NKG2DL targeting agent may, for example, be a “patient specific composition” that includes both a radionuclide labeled portion and a non-radiolabeled portion. The majority of the targeting agent (recombinant protein, antibody, peptide, oligonucleotide, small molecule, etc.) administered to a patient may typically consist of non-radiolabeled targeting agent, with the minority being the radiolabeled targeting agent. The ratio of labeled to non-labeled targeting agent may be adjusted using known methods. The patient specific composition may, for example, include the NKG2DL targeting agent in a ratio of radiolabeled:non-radiolabeled NKG2DL targeting agent of from about 0.01:10 to 1:1, such as 0.1:10 to 1:1 radiolabeled:non-radiolabeled.

The NKG2DL targeting agent (radiolabeled and non-radiolabeled collectively) may, for example, be provided in a total protein amount of up to 100 mg, such as up to 60 mg, such as 5 mg to 45 mg, or a total protein amount of from 0.001 mg/kg patient weight to 3.0 mg/kg patient weight, such as from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

The inventive combination of a radiolabeled fraction and a non-radiolabeled fraction of the antibody or other targeting agent (e.g., biologic delivery vehicle) allows the composition to be tailored to a specific patient, wherein each of the radiation dose and the protein dose of the antibody or other biologic delivery vehicle are personalized to that patient based on at least one patient specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container. One of the advantages of the disclosed composition is that there will be no left-over radiation that would need to be discarded or handled by the medical personnel, e.g., no dilution, or other manipulation to obtain a dose for the patient. When provided in a single dose container, the container is simply placed in-line in an infusion tubing set for infusion to the patient. Moreover, the volume can be standardized so that there is a greatly reduced possibility of medical error (i.e., delivery of an incorrect dose, as the entire volume of the composition is to be administered in one infusion).

Thus, the NKG2DL targeting agent may be provided as a single dose composition tailored to a specific patient, wherein the amount of radiolabeled and non-radiolabeled NKG2DL targeting agent in the composition may depend on one or more of a patient weight, age, gender, disease state and/or health status. The NKG2DL targeting agent may be provided as a multi-dose therapeutic, wherein each dose in the treatment regime is provided as a patient specific composition. In one aspect, the patient specific composition includes radiolabeled and non-radiolabeled NKG2DL targeting agent, wherein the amounts of each depend on one or more of patient weight, age, gender, disease state, and/or health status.

As used herein, the terms “subject” and “patient” are interchangeable and include, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells). “Treating” a subject afflicted with a cancer shall also include, without limitation, slowing the proliferation of and/or damaging and/or killing cancer cells of the patient's cancer present in the patient.

“Chemotherapeutic”, in the context of this disclosure, shall mean a chemical compound which inhibits or kills growing cells and which can be used or is approved for use in the treatment of cancer. Exemplary chemotherapeutic agents include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alkaloids, in particular vinblastine, vincristine, vindesine, vinflunine, and vinorelbine. Exemplary chemotherapeutics also include radiosensitizers that may synergize with the radioconjugated NKG2DL targeting agent, such as temozolomide, cisplatin, and/or fluorouracil.

“Therapeutically effective amount” or “effective amount” refers to an amount effective, at dosages and for periods of time necessary, to achieve a desired therapeutic result when the subject agent is used as a monotherapy or in combination or conjunction with one or more other therapeutic agents or treatment modalities. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the radioconjugated NKG2DL targeting agent that may deplete or cause a reduction in the overall number of cells expressing NKG2DLs, or may inhibit or slow the growth of tumors having NKG2DL expressing cells therein, or may reduce the overall tumor burden of tumors having NKG2DL expressing cells therein, or may reduce the overall tumor burden of a subject, or may slow the growth of tumors in a subject, or may induce antitumor immunity in a subject. As disclosed herein, the tumors may be liquid or solid tumors.

As used herein, “depleting”, with respect to cells expressing NKG2DLs, shall mean to lower the population of at least one type of cells, such as cancer cells of a type of cancer, that express or overexpress NKG2DLs. According to certain aspects of this disclosure, a decrease may be determined by comparison of the numbers of NKG2DL-positive cells in a tissue biopsy, such as from the solid tumor, before and after initiation of treatment with the radioconjugated NKG2DL targeting agent. According to certain aspects of this disclosure, a decrease may also be determined by overall tumor size. As such, and by way of example, a subject's tumor size may be considered to be depleted if the population of tumor cells is lowered, such as by at least 10%, 20%, 30%, 40%, 50%, 60%, 70%, 80%, 90% or 99%.

“Inhibits growth” refers to a measurable decrease or delay in the growth (or rate of growth) of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

The term “antitumor immunity” refers to the ability of the presently disclosed compositions and methods to promote an antitumor effect by activating T cells and/or B cells and/or other immune cell having anti-cancer/tumor cell activity. Such an antitumor effect may be confirmed through comparison of treated mice (i.e., treated with at least the radioconjugated NKG2DL targeting agents and optionally the CD47 or immune checkpoint blockades disclosed herein) having normal immune functions and those with impaired immune functions, such as impaired T cells and B cells (nude mice). Alternatively, or additionally, antitumor immunity may be confirmed by determining the fraction of CD45-, CD3-, and CD8-positive cells (CD8-positive T cells) among living cells using flow cytometry, wherein increased numbers of CD45-, CD3-, and CD8-positive cells are expected for treated tumor-bearing mice as compared to untreated mice. Alternatively, the effect can be confirmed by analyzing images of an excised tumor stained with an anti-CD8 antibody and counting the number of CD8-positive cells per unit area in the tumor to examine the increased number of CD45-, CD3-, and CD8-positive cells for treated as compared to untreated mice.

The term “immune checkpoint therapy” refers to a molecule capable of modulating the function of an immune checkpoint protein in a positive or negative way (in particular the interaction between an antigen presenting cell (APC) such as a cancer cell and an immune T effector cell). The term “immune checkpoint” refers to a protein directly or indirectly involved in an immune pathway that under normal physiological conditions is involved in preventing uncontrolled immune reactions and thus for the maintenance of self-tolerance and/or tissue protection. The one or more immune checkpoint therapies described herein may, for example, independently act at any step of the T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that fine tune the response.

In the context of the present disclosure, an immune checkpoint therapy encompasses therapies such as antibodies capable of down-regulating at least partially the function of an inhibitory immune checkpoint (antagonist) and/or up-regulating at least partially the function of a stimulatory immune checkpoint (agonist). For example, an immune checkpoint therapy may refer to an antagonist antibody against an immune checkpoint protein that may be upregulated in certain cancers, and thus may inhibit the function of the immune checkpoint protein (i.e., act as an immune checkpoint blockade).

The term “DDRi” refers to an inhibitor of a DNA damage response pathway protein, of which a PARPi is an example. The term “PARPi” refers to an inhibitor of poly(ADP-ribose) polymerase. In the context of the present disclosure, the term PARPi encompasses molecules that may bind to and inhibitor the function of poly(ADP-ribose) polymerase, such as antibodies, peptides, or small molecules.

The term “CD47 blockade” refers to an agent that prevents CD47 binding to SIRPα, such as agents that bind to either of CD47 or SIRPα, those that modulate expression of CD47 or SIRPα, or those that otherwise diminish the “don't eat me” activity of the CD47SIRPα axis. In the context of the present disclosure, CD47 blockades include at least antibodies that bind to CD47 such as magrolimab, lemzoparlimab, and AO-176, antibodies that bind to SIRPα, CD47-binding SIRPα Fc fusion proteins, agents that modulate the expression of CD47 and/or SIRPα, such as phosphorodiamidate morpholino oligomers (PMO) that block translation of CD47 such as MBT-001, and small molecule inhibitors of the CD47/SIRPα axis such as RRx-001.

As used herein, administering to a subject one or more additional therapies, such as one or more of an immune checkpoint therapy and/or DDRi and/or CD47 blockade and/or radiosensitizer “in combination with” or “in conjunction with” a radioconjugated NKG2DL targeting agent means administering the additional therapy before, during and/or after administration of the radioconjugated NKG2DL targeting agent. This administration includes, without limitation, the following scenarios: (i) the additional therapy is administered first, and the radioconjugated NKG2DL targeting agent is administered second; (ii) the additional therapy is administered concurrently with the radioconjugated NKG2DL targeting agent (e.g., the DDRi is administered orally once per day for n days, and the radioconjugated NKG2DL targeting agent is administered intravenously in a single dose on one of days 2 through n−1 of the DDRi regimen); (iii) the additional therapy is administered concurrently with the radioconjugated NKG2DL targeting agent (e.g., the DDRi is administered orally for a duration of greater than one month, such as orally once per day for 35 days, 42 days, 49 days, or a longer period during which the cancer being treated does not progress and during which the DDRi does not cause unacceptable toxicity, and the radioconjugated NKG2DL targeting agent is administered intravenously in a single dose on a day within the first month of the DDRi regimen); and (iv) the radioconjugated NKG2DL targeting agent is administered first (e.g., intravenously in a single dose or a plurality of doses over a period of weeks), and the additional therapy is administered second (e.g., the DDRi is administered orally once per day for 21 days, 28 days, 35 days, 42 days, 49 days, or a longer period during which the cancer being treated does not progress and during which the DDRi does not cause unacceptable toxicity).

An “article of manufacture” indicates a package containing materials useful for the treatment, prevention and/or diagnosis of the disorders described herein. The article of manufacture may include a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a radioconjugated NKG2DL targeting agent according to aspects of the present disclosure.

A “label” or “package insert” is used to refer to instructions customarily included in commercial packages of therapeutic products that contain information about the indications, usage, dosage, administration, combination therapy, contraindications and/or warnings concerning the use of such therapeutic products. As used herein, a label may indicate that the composition is used for treating a NKG2DL-positive cancer and may optionally indicate administration routes and/or methods. Moreover, the article of manufacture may include (a) a first container with a composition contained therein, wherein the composition includes a radioconjugated NKG2DL targeting agent; and (b) a second container with a composition contained therein, wherein the composition includes a further cytotoxic or otherwise therapeutic agent according to aspects of the present disclosure. Alternatively, or additionally, the article of manufacture may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Unless otherwise defined, all technical and scientific terms used herein have the same meaning as commonly understood by one of ordinary skill in the art to which the present disclosure belongs.

### Aspects of the Invention

In one aspect, the present invention provides novel radioconjugated agents that recognize NKG2DLs expressed on the surface of cancer cells, which may provide therapeutic efficacy against all cancer types—both liquid and solid tumors—that display NKG2DLs on the cell surface. The mechanism of action for eradication of primary and metastatic tumors involves targeted delivery of lethal radiation, such as from as low as a single radionuclide, to transformed cells and to adjacent diseased cells. This radioimmunotherapy approach, i.e., targeted recognition and binding of NKG2DLs by the disclosed radioconjugated targeting agents, is especially novel because radiation itself induces NKG2DLs. As such, the presently disclosed radioconjugated NKG2DL targeting agents may also lead to a feed-forward mechanism of tumor ablation. Whereas other NKG2DL-targeting approaches rely on immune activation (e.g., CAR T cells, bispecific engagement of tumor and immune cells), radioimmunotherapy directly kills cancer cells, circumventing current limitations of immunotherapy, i.e., suppression of T cell activation and lack of long-term persistence of the adoptively transferred cells.

Moreover, immunosuppressive cells within the tumor microenvironment are known to express NKG2DLs (Feng, 2020). Thus, radioconjugated NKG2DL-targeting agents could ablate these cells as well, leading to improved immune response against tumors. NK cells tend to infiltrate solid tumors to a lower extent compared to T cells, whereas NK cells are readily present within bone marrow and blood, which are sites of liquid tumor proliferation (Xing, 2020). Since radioimmunotherapy does not rely on immune cell recruitment, the presently disclosed inventions would be efficacious against both solid and liquid tumors without regard to differential tissue compartmentalization of the immune cells. Lastly, since NKG2DLs are expressed on a multitude of cancer cells, the presently disclosed inventions would be broadly applicable without needing to rely on individual tumor markers (e.g., HER2, EGFR, PSMA).

NKG2D fusion proteins have been generated in the past that form a physical link between the tumor cell and NK cell, leading to immune-mediated suppression of tumor growth (Ding, 2018). In one construct, the soluble portion of the NKG2D receptor (which recognizes the tumor cell expressing NKG2DL) was fused to the Fc portion of IgG, and this recombinant protein caused cell-mediated cytotoxicity against breast cancer cells, including those that expressed low levels of HER2 and were not amenable to trastuzumab treatment. Further optimization of the Fc region through S239D/I332E mutations enhanced the cytotoxic activity even more (Raab, 2014).

In another preclinical study, NKG2D-Fc elicited anti-tumor effect against leukemia but was non-responsive against normal blood cells, providing evidence that NKG2D-based therapeutics may safely target tumor cells while avoiding harm to healthy cells (Steinbacher, 2015). The NKG2D-Fc protein coated with iron oxide nanoparticles (which exhibit anticancer effect) has been shown to specifically transport the nanoparticles to NKG2DL-expressing cancer cell lines, demonstrating that recombinant NKG2D is useful for delivering therapeutic cargo (Wu, 2014).

In addition to directly targeting cancer cells, NKG2D-Fc protein may also indirectly enhance antitumor effect by removing immunosuppressive cells in the tumor microenvironment. For instance, in a murine model of colon adenocarcinoma, NKG2D-Fc was shown to bind to NKG2DL-expressing myeloid-derived suppressor cells (MDSCs) and regulatory T cells (Tregs) and reduced both the circulating and tumor-infiltrating levels of these immunosuppressive cells; in parallel, the circulating and tumor-infiltrating levels of NK cells increased (Feng, 2020). These combined changes likely contributed to the improved survival rate in tumor-bearing mice and suggest that an NKG2DL-targeting approach may be utilized to shift the tumor microenvironment to a more immunostimulatory state.

Non-Fc domain proteins may also be fused to NKG2D. For instance, NKG2D fused to anti-CD3 scFv can function like a bi-specific T cell engager (BiTE) by recruiting T cells to tumors expressing NKG2DLs. In a preclinical model of lymphoma, NKG2D-anti-CD3 scFv fusion protein was able to generate tumor-free survival and elicit host-specific immunologic memory that resisted rechallenge with tumor cells (Zhang, 2011). NKG2D may also be fused to cytokines for multifunctionality. NKG2D fused to TL-2 (produced from an intramuscularly administered DNA construct) was capable of delivering the immune-activating cytokine moiety to the tumor microenvironment, leading to enhanced T cell response and reduced tumor burden in a murine model. Fusion of TL-2 to NKG2D was beneficial for another reason because targeted delivery of TL-2 to the tumor avoided toxic side effects observed with systemic administration of free TL-2 (Kang, 2012). Anti-tumor effects were also observed when NKG2D was fused to IL-15 in a mouse model of colon cancer (Xia, 2014) and xenograft model of gastric cancer (Chen, 2017). Finally, NKG2D fused to IL-21 (expressed from a DNA construct loaded within chitosan nanoparticles for intramuscular administration) slowed tumor growth and prolonged survival in a syngeneic model of colon cancer (Tan, 2017).

In all of the examples described above, the NKG2D receptor portion of the fusion protein was exploited as a targeting agent to specifically localize the immune effector molecule (Fc, anti-CD3 scFv) or immunostimulatory cytokine (IL-2, IL-15, and IL-21) to the tumor. However, the binding affinity of NKG2D to human NKG2DLs ranges from 600-1100 nM (Nausch, 2008). Accordingly, the presently disclosed inventions also include antibodies or other engineered molecules (e.g., DARPins, Affimers, Aptamers, etc.) with higher binding affinities towards the NKG2DLs. Such NKG2DL targeting agents provide a therapeutically viable method for targeted deposition of radiation specifically within tumors.

One strategy of the present disclosure is to target the proteolytic cleavage site of NKG2DLs, such as on MICA and MICB. These NKG2DLs are expressed in multiple tumor types, including pancreatic carcinoma, hepatocellular carcinoma, melanoma, colorectal cancer, adrenal gland carcinoma, endometrial and ovarian tumors, and head and neck squamous cell carcinoma (Morel, 2016). Since proteolytic cleavage is an immune escape mechanism, an antibody that binds near the cleavage site and prevents MICA/B proteolysis—thereby retaining the cell surface localization of these NKG2DLs—would stimulate NK cell-directed tumor lysis.

The extracellular portions of MICA/B are composed of 3 domains: alpha-1, alpha-2, and alpha-3. Alpha-1/2 domains are recognized by NKG2D and are most distal from the plasma membrane, whereas the membrane proximal alpha-3 domain contains the cleavage site recognized by proteases, such as MMP14, ADAM10, and ADAM17. A monoclonal antibody (clone 7C6; mouse IgG2a and human IgG1) generated against this alpha-3 domain was shown to maintain MICA/B levels on cancer cell surfaces and, in an NK cell-dependent manner, reduced lung metastasis in a preclinical model of melanoma (De Andrade, 2018). A follow-up study demonstrated that this monoclonal antibody 7C6 inhibited metastasis even for cells that were resistant to T cell-mediated cell death as a consequence of lacking B2M/MHC expression (De Andrade, 2020). These studies further demonstrate that the presently disclosed inventions, which include therapeutics based on NKG2D/NKG2DLs, offer an alternative approach for patients whose tumors do not respond to other forms of immunotherapy.

When MICA/B do become cleaved, the soluble fragments can bind and mask the NKG2D receptor on NK cells, thereby preventing the recognition of tumors expressing NKG2DLs. To overcome this obstacle, another group showed that a different alpha-3 domain specific monoclonal antibody (6E1; human IgG1 chimera) can form a link between NK cells using cleaved, soluble MICA as an intermediary to stimulate immune response, suggesting that targeting the alpha-3 domain of MICA/B may have clinical benefits even in the presence of cleaved MICA (Du, 2019).

Other MICA/B antibodies that have been reported in preclinical studies include clone B10G5 (mouse IgG1), which inhibited the growth of prostate cancer (Lu, 2015; Basher, 2020), and clone IPH4301 (human IgG1), which demonstrated tumor reduction (Morel, 2016; Courau, 2019).

Accordingly, the therapeutic methods of the present disclosure include administration of a therapeutically effective amount of a radioconjugated NKG2DL targeting agent, such as a radiolabeled protein (e.g., soluble recombinant version of NKG2D), or a radiolabeled antibody, peptide, or small molecule that targets NKG2DL, either alone or in combination with an additional therapeutic agent or modality. The additional agent or modality may be any one or more of administration of an immune checkpoint therapy, a DDRi, a CD47 blockade, a chemotherapeutic agent, or radiation therapy (i.e., external beam radiation or brachytherapy).

The NKG2DL targeting agent may, for example, be administered to the patient in a patient specific composition in one or more doses.

The patient may be monitored at intervals during the therapy for the presence of NKG2DL positive cells to evaluate the reduction in NKG2DL-positive cells. Detecting a decreased number of the NKG2DL-positive cells after treatment with the NKG2DL targeting agent, as compared to the number of NKG2DL-positive cells prior to treatment may indicate effectiveness of the NKG2DL targeting agent in treating a NKG2DL-positive cancer in the mammalian subject.

The methods of treating cancer disclosed herein may include identifying a patient that has a NKG2DL-positive cancer by identifying NKG2DL-positive cells, and administering to the patient a therapeutically effective amount of a NKG2DL targeting agent, either alone or in combination with an additional therapeutic agents or modalities. The additional treatment may, for example, include any one or more of administration of an immune checkpoint therapy, a DDRi, a CD47 blockade, an HDAC inhibitor, an LSD1 inhibitor, a chemotherapeutic agent, or radiation therapy (i.e., external beam radiation or brachytherapy). The chemotherapeutic agent may be a radiosensitizer.

The radioconjugated NKG2DL targeting agent may, for example, be administered to a subject/patient that has also already undergone a previous treatment, such as surgery for treatment of the cancer, such as to remove all or a portion of a solid tumor.

### NKG2DL Targeting Agents

In one aspect, the present disclosure provides compositions including radiolabeled NKG2DL targeting agents, and methods of use thereof. Exemplary NKG2DL targeting agents that may be radiolabeled for use in or embodiment in the various aspects of the invention include, for example, recombinant soluble NKG2D receptors or anti-NKG2DL antibodies, recombinant proteins, small domain proteins such as a DARPin, anticalin, or affimer, or a peptide, aptamer, or small molecule that binds to one or more NKG2DLs. Examples of NKG2DL targeting agents that may be radiolabeled for use in or embodiment in the various aspects of the invention are set forth hereinbelow and throughout this disclosure.

NKG2DLs are structurally similar to MHC class I molecules. MICA and MICB have the same alpha-1, alpha-2, and alpha-3 domains as MHC class I molecules, in which the alpha-3 domain is an Ig-like domain. ULBP1-6 on the other hand have only alpha-1 and alpha-2 domains, where ULBP1, -2, -3, and -6 include GPI anchoring receptors, and ULBP4 and -5 have a transmembrane domain and cytoplasmic tail. NKG2DLs are polymorphic, with MICA having about 100 alleles and MICB having 40 alleles. Different isomers affect the expression of MICA and MICB and their affinity with NKG2D, and thus alter the effects of the NKG2D receptor\NKG2D ligand axis and NK cell activity.

Exemplary NKG2DL targeting agents include monoclonal antibodies against any of the cell surface regions of an NKG2DL, such as a monoclonal antibody or antigen-binding fragment thereof against any cell surface portion of MICA, MICB, or any one of ULBP1-6. The monoclonal antibodies may, for example, generally recognize and bind to the cell surface alpha-1 and/or alpha-2 domain, or in the case of MICA and MICB, may additionally or alternatively recognize and bind to the cell surface alpha-3 domain. Accordingly, the NKG2DL targeting agents may block interaction of NKG2D with at least one NKG2DL; and/or may block cleavage and shedding of an NKG2DL, such as cleavage at the alpha-3 domain of MICA/B; and/or, when bound to the NKG2DL, such as the alpha-3 domain of MICA/B, may not decrease recognition of MICA/B by natural killer (NK) cells by more than 40%, such as by not more than 30%, or 20%, or 10%.

An exemplary targeting agent may bind to any linear or conformational epitope of MICA, wherein MICA includes 274 amino acids. For example, the alpha-3 domain is located within amino acid residues 182 to 274, respectively. An exemplary antibody against the alpha-3 domain of MICA may bind an epitope including one, two or three residues selected from the group consisting of T227, Q228 and Q229; one, two or three residues selected from the group consisting of S224, H225 and D226; and/or one or two residues selected from the group consisting of W230 and D232. Exemplary antibodies against MICA include clones available commercially, such as 6D4, 1C2, 6F11, and IPH43. Clone 6D4 is a mouse IgG₂ₐ monoclonal antibody that specifically binds both human MICA and MICB (Catalog No. 320902, BioLegend UK Ltd, London, UK).

An exemplary antibody against MICA includes clone 7C6 (and any of those disclosed in U.S. Pub. No. 20200165343) that specifically targets the alpha-3 domain of MICA. For example, the antibody may include light chain complementarity determining regions (CDRS) having the amino acid sequences: CDR1 (SEQ ID NO:1) SASQDISNYLN, CDR2 (SEQ ID NO:2) DTSILTHL, CDR3 (SEQ ID NO:3) QQYSKFPRT; and heavy chain CDRS having the amino acid sequences: CDR1 (SEQ ID NO:4) NYAMN, CDR2 (SEQ ID NO:5) WINTHTGDPTYADDFKG, CDR3 (SEQ ID NO:6) TYGNYAMDY. The antibody against MICA may, for example, include a light chain variable region having the amino acid sequence (SEQ NO:7):

DIQMTQTTSSLSASLGDRVTISCSASQDISNYLNWYQQKPDGTVK
LLIYDTSILHLGVPSRFSGSGSGTDYSLTISNLEPEDIATYYCQQ
YSKFPRTFGGGTTLEIK,

and a heavy chain variable region having the amino acid sequence (SEQ NO:8):

QIQLVQSGPELKKPGETVKVSCKASGYMFTNYAMNWVKQAPEKGL
KWMGWINTHTGDPTYADDFKGRIAFSLETSASTAYLQINNLKNED
TATYFCVRTYGNYAMDYWGQGTSVTVSSAKTTAPSVYPLAPVCGD
TTGSSVTLGCLVKGYFPEPVTLTWNSGSLSSGVHTFPAVLQSDLY
TLSSSVTVTSS.

An exemplary antibody against MICA includes an isolated antibody or antigen-binding portion thereof that specifically targets the alpha-3 domain of MICA, such as any of those disclosed in European Pub. No. EP3077504 or U.S. Pat. No. 10,106,611. For example, the antibody against MICA may include a light chain variable region having the amino acid sequence (SEQ NO:9):

DIQLTQSPSFLSASVGDRVTITCRASQGITSYLAWYQQKPGKAPK
LLIYAASALQSGVPSRFSGRGSGTEFTLTISSLQPEDFATYYCQQ
VNRGAAITFGHGTRLDIKR,

and a heavy chain variable region having the amino acid sequence (SEQ NO:10):

QVQLVQSGAEVKKPGSSVRXSCRASGGSSTTYAFXWVRQAPGQGL
EWMGGIVPIFGTLKYAQKFQDRVTLTADKSTGTAYMELNSLRLDD
TAVYYCARAIQLEGRPFDHWGQGTQVTVSA.

Another exemplary antibody against MICA includes an isolated antibody or antigen-binding portion thereof that specifically targets the alpha-3 domain of MICA, such as of those disclosed in U.S. Pat. No. 8,753,640. For example, the antibody may include light chain complementarity determining regions (CDRs) having the amino acid sequences: CDR1 (SEQ ID NO:11) QASQDIGNNLI, CDR2 (SEQ ID NO:12) YATNLAN, CDR3 (SEQ ID NO:13) QQWSSNP; and heavy chain CDRS having the amino acid sequences: CDR1 (SEQ ID NO:14) NYYMS, CDR2 (SEQ ID NO:15) NIYGGNGGTGYNQKFKG, CDR3 (SEQ ID NO:16) GDLYAMDY. The antibody may, for example, include a light chain including the amino acid sequence (SEQ ID NO:17):

VLTQSPSSMSASLGDRVTITCQASQDIGNNLIWFQQKPGKSPRPM
IYYATNLANGVPSRFSGSGSGTSYSLTISSMEAEDAATYYCQQWS
SNPYTFGG

and a heavy chain including the amino acid sequence (SEQ ID NO:18):

AELVKPGASVKLSCKTSGYTFSNYYMSWLKQMPGQNIEWIGNIYG
GNGGTGYNQKFKGKATLTVDKSSSTAYMQLSSLTSEDSAVYFCAR
GDLYAMDYWGQGTTVT.

The antibody may, for example, include an scFv including the amino acid sequence (SEQ ID NO:19):

MAQVQLQQSGAELVKPGASVKLSCKTSGYTFSNYYMSWLKQMPGQ
NIEWIGNIYGGNGGTGYNQKFKGKATLTVDKSSSTAYMQLSSLTS
EDSAVYFCARGDLYAMDYWGQGTTVTVSSGGGGSGGGGSGGGGSD
IVLTQSPSSMSASLGDRVTITCQASQDIGNNLIWFQQKPGKSPRP
MIYYATNLANGVPSRFSGSGSGTSYSLTISSMEAEDAATYYCQQW
SSNPYTFGGGTKLEIKRAAA.

Exemplary antibodies against MICA include clone 14B4 and any of those disclosed in U.S. Pat. No. 10,577,416 that specifically target the alpha-3 domain of MICA. For example, the antibody may include light chain complementarity determining regions (CDRs) having the amino acid sequences: CDR1 (SEQ ID NO:20) RASQNIDTSIH, CDR2 (SEQ ID NO:21) YASESIS, CDR3 (SEQ ID NO:22) QQSNYWPLT; and heavy chain CDRS having the amino acid sequences: CDR1 (SEQ ID NO:23-25) SYWMN, GYSFTS or GYSFTSYWMN, CDR2 (SEQ ID NO:26-27) MIIIPSDSETRLNQKFKD or MIHPSDSETR, CDR3 (SEQ ID NO:28) EMGPYTLDY. The antibody may, for example, include a light chain variable region having the amino acid sequence (SEQ ID NO:29):

MSVPTQVLGLLLLWLTDARCDILLTQSPAILSVSPGARVSFSCRA
SQNIDTSIHWYQQRTNGSPRLLIKYASESISGIPSRFSGSGSGTD
FTLSINSVESEDIADYYCQQSNYWPLTFGAGTKLELK,

and a heavy chain variable region having the amino acid sequence (SEQ ID NO:30):

MEWSWVFLFFLSVTTGVHSQVQLQQPGAELVRPGASVKLSCKASG
YSFTSYWMNWMKQRPGQGLEWIGMIHPSDSETRLNQKFKDKATLT
VDKSSSTAYMQLNSPTSEDSAVYYCAREMGPYTLDYWGQGTSVTV
SSASTK.

Exemplary antibodies against MICA include clone 16A8 and any of those disclosed in U.S. Pat. No. 10,577,416 that specifically target the alpha-3 domain of MICA. For example, the antibody may include light chain complementarity determining regions (CDRs) having the amino acid sequences: CDR1 (SEQ ID NO:31) KSSQSLLNSSNQKNYL, CDR2 (SEQ ID NO:32) FASTRES, CDR3 (SEQ ID NO:33) QQHYSTPPT; and heavy chain CDRS having the amino acid sequences: CDR1 (SEQ ID NO:34-36) RYAMS, GFTFSR or GFTFSRYAMS, CDR2 (SEQ ID NO:37-38) TIFSGGSYTYYPDSV or TIFSGGSY, CDR3 (SEQ ID NO:39) PNWERTFDY. The antibody may, for example, include a light chain variable region having the amino acid sequence (SEQ ID NO:40):

MESQTQVLMFLLLWVSGACTDIVMTQSPSSLAMSVGQKVTMSCKS
SQSLLNSSNQKNYLAWYQQKPGQSPKLLVYFASTRESGVPDRFMG
SGSGTDFTLTISSVQAEDLADYFCQQHYSTPPTFGGGTKLEIK.

and a heavy chain variable region having the amino acid sequence (SEQ ID NO:41):

MRVLILLWLFTAFPGLLSDVQLQESGPGLVKPSQSLSLTCTVTG
YSITSDYAWNWIRQFPGNKLEWMGFVSYSGTTKYNPSLKSRISIT
RDTSENQFFLQLNSVTSEDTATYYCARGYGFDYWGQGTTLTVSS.

Exemplary antibodies against MICA include clone 1D5 and any of those disclosed in U.S. Pub. No. 20200055939 that specifically targets the alpha-3 domain of MICA. For example, the antibody may include light chain complementarity determining regions (CDRs) having the amino acid sequences:

CDR1
(SEQ ID NO: 42)
EIILTQSPTTMAASPGEKITITCSASSSISSHYLHWYQ,
CDR2
(SEQ ID NO: 43)
QKSGFSPKLLYRTSNLASGVPARESGSGSGTSYSLTIGTM,
CDR3
(SEQ ID NO: 44)
EAEDVATYYCQQGSSLPLTFGAGTK VEIK;

and heavy chain CDRs having the amino acid sequences:

CDR1
(SEQ ID NO: 45)
EIQLQQSGPELVKPGASVKVSCKASGYAFTSQNIYWVKQSH,
CDR2
(SEQ ID NO: 46)
GKSLEWIGYEPYNVVPMYNPKFKGKATLTVDKSSSSAYIH,
CDR3
(SEQ ID NO: 47)
LNSLTSEDSAIYYCARSGSSNFDYWGQGTTLTVSS.

The antibody may, for example, include CDRs defined by a stretch of at least 5 amino acids from each of the sequences listed hereinabove for clone 1D5, or may include CDRs having one to five amino acids substituted by a different amino acid, or added within the sequence, or omitted.

It should be understood that a NKG2DL-targeting agent that is radiolabeled for use in the invention, such as an antibody (e.g., a monoclonal antibody or an antigen-binding fragment thereof), can bind the NKG2DL, such as MICA or MICB, which is bound to or associated with the plasma membrane at the cell surface (at/on the extracellular face of the cell). Thus, in one aspect, the NKG2DL targeting agent that is radiolabeled for use in the invention can bind its NKG2DL target when the target is bound to or associated with the expressing cell's plasma membrane and is not a NKG2DL targeting agent that exclusively binds a soluble, extracellular non-plasma membrane bound or non-plasma membrane associated form of the NKG2DL. The NKG2DL targeting agent that is radiolabeled for use in the invention may or may not also bind a soluble, extracellular non-plasma membrane bound or non-plasma membrane associated form of the NKG2DL.

Additional targeting agents, such as monoclonal antibodies, specific for MICA and/or MICB that may be radiolabeled for use in or embodiment in the various aspects of the invention include, for example, any of those disclosed in any of: U.S. Pat. Nos. 10,793,633; 10,106,611; and 11,242,393; or in any of U.S. Pub. Nos. 20170267764; 20170198054; 20200299380; 20140037630; 20210253711; 20210139593; and 20200055939.

Targeting agents, such as monoclonal antibodies, that may be radiolabeled for use in or embodiment in the various aspects of the invention also include, for example, any of the anti-ULBP1, anti-ULBP2, or anti-ULBP3 monoclonal antibodies disclosed in U.S. Pat. No. 7,427,669 or U.S. Pat. No. 7,807,796, and any of the RAETIG specific monoclonal antibodies disclosed in U.S. Pub. No. 20080038248. Anti-(human ULBP1) monoclonal antibodies that may be used in or embodied in the various aspects of the invention are also commercially available, such as Catalog No. MAB1380 (monoclonal mouse IgG2A Clone #170818) from R&D Systems, ULBP1 Monoclonal Antibody Clone 170818 (Catalog #MA5-23882) and Clone 3A6F11 (Catalog #. MA5-38655 from Invitrogen (a brand of Thermo Fisher Scientific, Waltham, MA). Uniprot accession no. Q9BZM6 (SEQ ID NO:90) provides the amino acid sequence of human ULBP1 (UL16-binding protein 1); amino acids 26-217 make up the mature protein, amino acids 217-244 are absent in the mature protein, amino acids 1-25 make up an N-terminal signal sequence. Accordingly, suitable anti-UBLP1 antibodies that may be radiolabeled for use in or embodiment in the various aspects of the invention may, for example, be generated against a polypeptide having the amino acid sequence from residues 26 to 217 (of SEQ ID NO:90) or a subsequence therein.

The various sequences listed above that may define an antibody against MICA, which may be radiolabeled for use in or embodiment in the various aspects of the invention, may include any of:
(a) one or more of the CDRS of the light chain;
(b) one or more of the CDRS of the heavy chain;
(c) any combination of one or more CDRs from the light chain and one or more CDRs from the heavy chain;
(d) one or more CDRs from the light chain and the variable region of the heavy chain, wherein one to five amino acids from the heavy chain variable region may be substituted by a different amino acid; or
(e) the light chain variable region, wherein one to five amino acids from the light chain variable region may be substituted by a different amino acid, and one or more of the CDRs from the heavy chain.

It should be understood that wherever in this disclosure specific antibodies, specific antibody heavy chains and specific antibody light chains are disclosed, against a NKG2D ligand such as MICA or against any target, also intended to be disclosed for embodiment in or use in the various aspects of the invention (e.g., as radiolabeled NKG2DL targeting agents) are antibodies, such as but not limited to immunoglobulins, such as but not limited to IgG, that (i) include the heavy chain variable region of the disclosed antibody or heavy chain, (ii) include 1, 2 or 3 of the heavy chain CDRs (e.g., by Kabat definition) of the disclosed antibody or heavy chain, (iii) include the light chain variable region of the disclosed antibody or light chain, and/or (iv) include 1, 2 or 3 of the light chain CDRs (e.g., by Kabat definition) of the disclosed antibody or light chain. It should also be understood that wherever in this disclosure an antibody heavy chain or an antibody light chain is disclosed that includes an N-terminal leader sequence (or signal sequence), also intended to be disclosed for embodiment in and use in the various aspects of the invention are corresponding heavy chains and corresponding light chains that lack the leader sequence (or signal sequence). Further, where non-human monoclonal antibodies are exemplified as agents, also disclosed and provided by the invention are chimeric (i.e., part human; e.g., having a human Fc region) versions as well as humanized versions of said antibodies for use in or embodiment in the various aspects of the invention, for example, as radiolabeled NKG2DL targeting agents.

It is also possible that certain isomeric amino acid replacements with exact mass, such as Leu for Ile or vice versa, could be allowed in any of the sequences indicated herein. Additionally, certain portions of these sequences may be substituted, such as by related portions from human immunoglobulins to form chimeric immunoglobulins (i.e., chimeric or humanized ant-NKG2DLs). Exemplary substitutions include all or portions of the human leader sequence, and/or the conserved regions from human IgG1, IgG2, or IgG4 heavy chains and/or human Kappa light chain.

The NKG2DL targeting agent may, for example, be monospecific having specificity to only one epitope of a selected NGKDL, or to only one epitope shared across more than one NKG2DL.

The NKG2DL targeting agent may, for example, be a multispecific antibody against a first epitope of an NKG2DL and at least a second epitope of the same NKG2DL, or a different NKG2DL, or a different (i.e., second) antigen that is not a NKG2DL, such as a cancer-associated antigen.

The NKG2DL targeting agent may, for example, be a mixture of an antibody against an epitope of an NKG2DL and one or more antibodies against a different epitope of the same NKG2DL, or a different NKG2DL, or a different (i.e., second) antigen that is not a NKG2DL, such as a cancer-associated antigen.

The additional different or second antigen may, for example, be an antigen differentially expressed on cells involved in various diseases or disorders such as cancer cells and/or precancerous cells, and/or on cells involved in solid tumors and/or immune suppression (such as MDSCs or Treg cells). For example, the additional different or second antigen may be a mammalian, such as human, form of CD33, DR5, 5T4, HER2 (ERBB2; Her2/neu), HER3, TROP2, mesothelin, TSHR, CD19, CD123, CD22, CD30, CD45, CD171, CD138, CS-1, CLL-1, GD2, GD3, B-cell maturation antigen (BCMA), Tn Ag, prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), LYPD3 (C4.4A), Nectin-4, urokinase plasminogen activator receptor (uPAR), Folate receptor alpha (FOLR1), CUB-domain containing protein 1 (CDCP1), Glypican-3 (GPC3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, phosphatidylserine, GRP78 (BiP), TAG72, CD38, CD44v6, CEA, EPCAM, B7H3, KIT, IL-13Ra2, interleukin-11 receptor a (IL-1 1Ra), PSCA, PRSS21, VEGFR2, LewisY, CD24, platelet-derived growth factor receptor-beta (PDGFR-beta), SSEA-4, CD20, Folate receptor alpha (FRa), MUCI, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, DR5, 5T4, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WTi, NY-ESO-1, LAGE-1a, MAGE-A1, legumain, HPV E6, E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLECi2A, BST2, EMR2, LY75, GPC3, FCRL5, GPA7, IGLL1, FGFR2, FGFR2b, Six-transmembrane epithelial antigen of prostate 1 (STEAP1), MUC17, claudin-18 isoform 2 (CLDN18.2), and Sortilin (Neurotensin receptor-3).

Additional exemplary NKG2DL targeting agents that may be radiolabeled and used in or embodied in the various aspects of the invention include a soluble recombinant NKG2D receptor, which is unique in that it may broadly target a tumor that expresses any of the NKG2DLs, leveraging the endogenous function of the receptor and obviating the need for additional alteration to the ligand binding region of the receptor (Song, 2013). The NKG2D receptor only binds its ligands with moderate affinities, however, and must be dimerized, such as homodimerized, or multimerized generally, to effectively bind its ligands. Accordingly, in one aspect, soluble recombinant NKG2D receptors of the present disclosure include at least two NKG2D monomers within a construct to form an NKG2D homodimer (or homomultimer) that can effectively bind an NKG2DL. Such a construct may bind the NKG2DL whether cell bound or shed, and even when the shed NKG2DL is in the circulation. For example, a fusion protein including the Fc region (Pro 100-Lys 300) of human IgG1 Uniprot accession no. P01857 (SEQ ID NO: 48) and the extracellular domain (Ile 73-Val 216) of human NKG2D isoform-1 Uniprot accession no. P26718-1 (SEQ ID NO: 49), wherein the Fc region is N-terminal to the NKG2D portion, may be used. A linker amino acid sequence such as IEGR (SEQ ID NO:50) may be disposed between the Fc portion and the NKG2D portion. A further two amino acid sequence, MD (SEQ ID NO:51), may optionally be disposed at/as the N-terminus of the fusion protein. Recombinant human NKG2D-Fc chimeric fusion proteins that may be used are commercially available, such as Catalog No. 1299-NK from R&D Systems (Minneapolis, MN, USA) and Catalog No. NKD-H5265 from Acro Biosystems (Newark, DE, USA), each having a disulfide-linked homodimer structure. NKG2D-Fc fusion proteins having Fc regions optimized to induce NK-cell reactivity or to minimize or knock out NK-cell reactivity, as disclosed for example, in Steinbacher et al., Int J Cancer 2015 Mar. 1; 136(5): 1073-84, may also be radiolabeled for use in or embodiment in the various aspects of the invention. Further, any of the NKG2D-Fc chimeric proteins disclosed in U.S. Pat. No. 10,865,232 or U.S. Pub. No. 20210130434 may be radiolabeled for use in or embodiment in the various aspects of the present invention.

The NKG2DL targeting agent may, for example, be a peptide or small molecule that binds to an NKG2DL. For example, it is contemplated that the NKG2DL targeting agent may be a glycoprotein, carbohydrate, lipid, or protein bound lipid. Carbohydrates may be natural or synthetic. A carbohydrate may be a derivatized natural carbohydrate. The carbohydrate may be a polysaccharide, such as, but not limited to, pullulan, cellulose, microcrystalline cellulose, hydroxypropyl methylcellulose (HPMC), hydroxvcellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin. konjac, glucommannan, pustulan, heparin, hyaluronic acid, curdlan, and xanthan. The carbohydrate may be a sugar alcohol, such as, but not limited to mannitol, sorbitol, xylitol, erythritol, maltitol, or lactitol.

While the NKG2DL targeting agents have been described as either recombinant proteins or monoclonal antibodies, any agent that specifically binds to one or more NKG2DLs—regardless of the biological or chemical form—is envisioned as within the scope of the presently disclosed invention and may yield therapeutic benefit when conjugated with a radionuclide as disclosed herein.

Methods of Treatment with NKG2DL Targeting Agents

One object of the present disclosure is to provide radioconjugated NKG2DL targeting agents useful in diagnostic assays and/or effective in therapeutic interventions, such as for the treatment of hematological (liquid) cancers and/or solid tumors. Mechanisms by which the presently disclosed radioconjugated NKG2DL targeting agents can effect a therapeutic response include targeted DNA damage from the ionizing radiation provided by the radiolabel.

In one aspect, the present disclosure provides methods of treating a proliferative disease or disorder in a mammalian subject such as a human that include administration of an effective amount of a radiolabeled NKG2DL targeting agent to the subject, alone or in combination or conjunction with one or more additional therapeutic agents or treatment modalities. In another aspect, the present disclosure provides methods of treating a proliferative disease or disorder in a mammalian subject such as a human that includes administration of an effective amount of a radiolabeled multispecific antibody against two or more epitopes of the same or different NKG2DLs, or against an epitope of a NKG2DL and an against one or more additional different (non-NKG2DL) antigens.

In a further aspect, the present disclosure provides methods of treating a proliferative disease or disorder in a mammalian subject such as a human which includes administration of a first antibody against at least one NKG2DL, and administration of a second antibody, wherein the second antibody is against a different epitope of the same or a different NKG2DL than the first antibody, or is against an epitope of a different antigen, such as an antigen selected from the list presented above, wherein either or both of the first antibody or the second antibody is radiolabeled. When both antibodies, or more than one different antibodies generally, are radiolabeled, they may be radiolabeled with the same radionuclide(s) or different radionuclide(s), such as any of those disclosed herein.

Such combinations, presented as a multispecific antibody or more than one monoclonal antibody as indicated above, may deliver a synergistic therapeutic effect.

When the methods include administration of a multispecific antibody, the first target recognition component may, for example, include one of: a first full length heavy chain and a first full length light chain, a first Fab fragment, or a first single-chain variable fragment (scFvs). The second target recognition component may, for example, include one of: a second full length heavy chain and a second full length light chain, a second Fab fragment, or a second single-chain variable fragment (scFvs). Moreover, the second target recognition component may, for example, be derived from a different epitope of the NKG2DL antigen or may be derived from any of the antigens listed above.

The NKG2DL targeting agent can be radiolabeled with a radioisotope, and any additional antibodies against other antigens may optionally be radiolabeled. When the immunotherapy includes a radiolabeled bispecific antibody, either one or both of the first target recognition component and the second target recognition component or any part of the bispecific antibody may include a radioisotope.

The radioconjugated NKG2DL targeting agent may exhibit essentially the same reactivity (e.g., immunoreactivity) to the antigen as a control targeting agent, wherein the control targeting agent includes a non-radiolabeled targeting agent against the same epitope of the antigen (i.e., NKG2DL) as the radiolabeled targeting agent.

The targeting agent may, for example, be labeled with ²²⁵Ac, and may be at least 5-fold more effective at causing cell death of NKG2DL-positive cells than a control monoclonal antibody, wherein the control monoclonal antibody includes an un-labeled antibody against the same epitope of the antigen as the ²²⁵Ac labeled antibody. For example, a ²²⁵Ac labeled monoclonal antibody may be at least 10-fold more effective, at least 20-fold more effective, at least 50-fold more effective, or at least 100-fold more effective at causing cell death of NKG2DL-positive cells than the control monoclonal antibody.

The methods may, for example, include administration of radiolabeled and non-radiolabeled (e.g., “naked”) fractions of the NKG2DL targeting agent, such as an antibody, antibody fragment, etc. For example, the non-radiolabeled fraction may include the same antibody against the same epitope as the radiolabeled fraction. In this way, the total radioactivity of the antibody composition may be varied or may be held constant while the overall antibody protein concentration may be held constant or may be varied, respectively. For example, the total protein concentration of non-radiolabeled antibody fraction administered may vary depending on the exact nature of the disease to be treated, age and weight of the patient, identity of the monoclonal antibody, and the label (e.g., radionuclide) selected for labeling of the monoclonal antibody. Any other radiolabeled targeting agent compositions used in or embodied in the various aspects of the invention may, for example, similarly include a radiolabeled portion and a non-radiolabeled portion of the targeting agent.

The effective amount of the radioconjugated NKG2DL targeting agent may, for example, be a maximum tolerated dose (MTD) of the radioconjugated NKG2DL targeting agent, such as an antibody against NKG2DL.

When more than one NKG2DL targeting agent or other immunotherapy is administered, the agents/antibodies may, for example, be administered at the same time. As such, the agents/antibodies may, for example, be provided in a single composition. Alternatively, the two agents/antibodies may be administered as separate compositions, for example, sequentially. As such, the radioconjugated NKG2DL targeting agent may be administered before the second agent/antibody, after the second agent/antibody, or both before and after the second agent/antibody. Moreover, the second agent/antibody may be administered before the radioconjugated NKG2DL targeting agent, after the radioconjugated NKG2DL targeting agent, or both before and after the radioconjugated NKG2DL targeting agent.

The radioconjugated NKG2DL targeting agent may, for example, be administered according to a dosing schedule selected from the group consisting of one every 7, 10, 12, 14, 20, 24, 28, 35, and 42 days throughout a treatment period, wherein the treatment period includes at least two doses.

The radioconjugated NKG2DL targeting agent may, for example, be administered according to a dose schedule that includes 2 doses, such as on days 1 and 5, 6, 7, 8, 9, or 10 of a treatment period, or days 1 and 8 of a treatment period.

Administration of the radioconjugated NKG2DL targeting agents of the present disclosure, in addition to other therapeutic agents, may be performed in a number of ways depending upon whether local or systemic treatment is desired and upon the area to be treated. Administration may, for example, be intratracheal, intranasal, epidermal and transdermal, oral or parenteral. Parenteral administration includes intravenous, or intra-arterial, or subcutaneous, or intraperitoneal or intramuscular injection or infusion; or intracranial, e.g., intrathecal or intraventricular, administration. In some embodiments a slow-release preparation including the targeting agents(s) and/or other therapeutic agents may be administered. The various agents may be administered as a single treatment or in a series of treatments that continue as needed and for a duration of time that causes one or more symptoms of the cancer to be reduced or ameliorated, or that achieves another desired effect.

The dose(s) may vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents can be administered to an animal (e.g., a human) at a relatively low dose at first, with the dose subsequently increased until an appropriate response is obtained.

The radioconjugated NKG2DL targeting agent may, for example, be administered simultaneously or sequentially with the one or more additional therapeutic agents. Moreover, when more than one additional therapeutic agent is included/used, the additional therapeutic agents may be administered simultaneously and/or sequentially with each other and/or with the radioconjugated NKG2DL targeting agent.

### Radiolabeling the NKG2DL Targeting Agent

The NKG2DL targeting agents of the present disclosure are labeled with a radioisotope, i.e., radioconjugated. The NKG2DL targeting agent may be a protein or antibody against a NKG2DL that is recombinant and/or engineered to include one or more specific conjugation sites for a bifunctional chelator molecule. For example, when the NKG2DL targeting agent is an antibody against a NKG2DL, the antibody may be deglycosylated in the constant region, such as at asparagine-297 (Asn-297, N297; kabat number) in the heavy chain CH2 domain, for the purpose of uncovering a unique conjugation site, glutamine (i.e., Gln-295, Q295) so that it is available for conjugation with bifunctional chelator molecules which may then chelate the radionuclide.

The NKG2DL targeting agent may, for example, be an antibody against an NKG2DL that may have reduced disulfide bonds such as by using reducing agents, which may then be converted to dehydroalanine for the purpose of conjugating with a bifunctional chelator molecule.

The NKG2DL targeting agent may, for example, be an antibody against an NKG2DL that may have reduced disulfide bonds, such as by use of reducing agents, followed by conjugation via aryl bridges with a bifunctional chelator molecule. For example, a linker molecule such as 3,5-bis(bromomethyl)benzene may bridge the free sulfhydryl groups on the NKG2DL targeting agent.

The NKG2DL targeting agent may, for example, be an antibody against an NKG2DL that may have certain specific existing amino acids replaced with cysteine(s) that then can be used for site-specific labeling (e.g., conjugation with a bifunctional chelator molecule which may then chelate the radionuclide).

The NKG2DL targeting agents may be radiolabeled through site-specific conjugation of suitable bifunctional chelators. Exemplary chelator molecules that may be used include p-SCN-Bn-DOTA, NH₂-DOTA, NH₂—(CH₂)₁₋₂₀-DOTA, NH₂-(PEG)₁₋₂₀-DOTA, HS-DOTA, HS—(CH₂)₁₋₂₀-DOTA, HS-(PEG)₁₋₂₀-DOTA, dibromo-S—(CH₂)₁₋₂₀-DOTA, dibromo-S-(PEG)₁₋₂₀-DOTA, p-SCN-Bn-DOTP, NH₂-DOTP, NH₂—(CH₂)₁₋₂₀-DOTP, NH₂-(PEG)₁₋₂₀-DOTP, HS-DOTP, HS—(CH₂)₁₋₂₀-DOTP, HS-(PEG)₁₋₂₀-DOTP, dibromo-S—(CH₂)₁₋₂₀-DOTP, and dibromo-S-(PEG)₁₋₂₀-DOTP.

The chelator molecules may, for example, be attached to the NKG2DL targeting agent through a linker molecule. Exemplary linker molecules generally include:

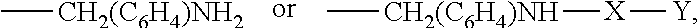

wherein X is

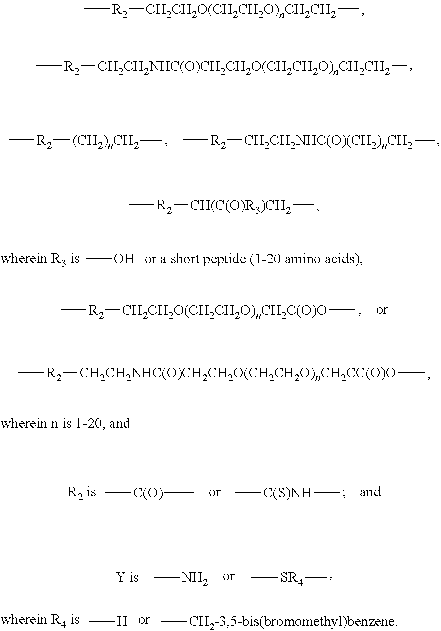

The NKG2DL targeting agents may be conjugated/labeled with any of the radioisotopes disclosed herein. According to certain aspects, the NKG2DL targeting agents are radioconjugated with ¹⁷⁷Lu [Lutetium-177], ²¹³Bi [Bismuth-213], ¹³¹I [Iodine-131]), or ²²⁵Ac. ²²⁵Ac exhibits a favorable profile for conjugation to biologics that target tumors.

²²⁵Ac is a radionuclide that emits alpha particles with high linear energy transfer (80 keV/μm) over a short distance (50-100 μm). The clusters of double strand DNA breaks that result after exposure to alpha particles are much more difficult to repair than damage from radionuclides that emit beta particles with low linear energy transfer (0.2 keV/μm). The inability to repair the extensive DNA damage eventually leads to cancer cell death. This potency of alpha particles can be exploited for targeted radioimmunotherapy, whereby ²²⁵A is conjugated to an antibody via a chelator. In this way, lethal radiation can be delivered specifically to cells bearing the target (e.g., tumor marker), allowing precise ablation of tumor cells while minimizing damage to healthy tissues. Furthermore, the long half-life of ²²⁵Ac (10 days) makes this radionuclide particularly attractive for therapeutic evaluation. ²²⁵Ac can be conjugated to any biologic (e.g., full-length antibody, scFv, Fab, NKG2D fusion protein, peptide) via a linker-chelator moiety, and in preclinical and clinical studies, dodecane tetraacetic acid (DOTA) is commonly used to stably chelate ²²⁵Ac, although other chelating agents may be used (see the section titled “Radiolabeling the NKG2DL targeting agent”).

Exemplary methods for conjugation and chelation of an exemplary radionuclide are described in more detail in Example 1.

### Diagnostic Aspects

The presently disclosed methods may further include steps for diagnosing the subject to ascertain if NKG2DL-positive cells are present in the patient such as in the circulation or within the tumor microenvironment, such as present in a tumor biopsy from the subject. The diagnosing step may generally include obtaining a sample of tissue from the subject and mounting the sample on a substrate. The presence or absence of the NKG2DL-positive cells may be detected using a diagnostic antibody, peptide, or small molecule, wherein the diagnostic antibody peptide, or small molecule is labeled with any of the standard imaging labels known in the art. Exemplary labeling agents include, for example, radiolabels such as ³H, ¹⁴C, ³²P, ³⁵S, and ¹²⁵I; fluorescent or chemiluminescent compounds, such as fluorescein isothiocyanate, rhodamine, or luciferin; and enzymes, such as alkaline phosphatase, β-galactosidase, or horseradish peroxidase used, for example, with colorimetric, fluorometric or chemiluminescent substrates therefor as known in the art. An exemplary NKG2DL targeting agent used in such a diagnostic assay may, for example, include a monoclonal or polyclonal antibody against any of the human NKG2DLs, such as MICA.

Alternatively, the methods may include diagnosing the subject to ascertain if NKG2DL-positive cells are present, to what extent they may be present and their localization within the subject, using an NKG2DL targeting agent labeled with any of ¹⁸F, ¹¹C, ⁶⁸Ga, ⁶⁴Cu, ⁸⁹Zr, ¹²⁴I, ⁴⁴Sc, or ⁸⁶Y, which are useful for PET imaging, or ⁶⁷Ga, ^{99m}Tc, ¹¹¹In, or ¹⁷⁷Lu, which are useful for SPECT imaging. Accordingly, the method may include administering to the subject an NKG2DL targeting agent labeled with one or more of ¹⁸F, ¹¹C, ⁶⁸Ga, ⁶⁴Cu, ⁸⁹Zr, ¹²⁴I, ⁴⁴Sc, ⁸⁶Y, ^{99m}Tc, ¹⁷⁷Lu, or ¹¹¹In, and performing a non-invasive imaging technique on the subject, such as performing a PET or SPECT scan on the subject. The method may include, performing the imaging after a sufficient amount of time, such as at least 30 minutes or at least 60 minutes, from the administration for the NKG2DL targeting agent to distribute to and bind to NKG2DL that may be present in the tissues of the subject (such as at tumor sites). The NKG2DL targeting agent for such imaging may, for example, include any of ¹⁸F, ¹¹C, ⁶⁸Ga, ⁶⁴Cu, ⁸⁹Zr, ¹²⁴, ⁴⁴Sc, ⁸⁶Y, ^{99m}Tc, ¹⁷⁷Lu, or ¹¹¹In, such as any of ⁶⁸Ga, ⁸⁹Zr, or ¹¹¹In, and may be labeled using any suitable methods (e.g., such as those disclosed in Example 1).

If the subject has NKG2DL-positive/over-expressing cells/tumor, the treatment methods of the present disclosure may be carried out, i.e., administration of a therapeutically effective amount of a radioconjugated NKG2DL targeting agent (e.g., ²²⁵Ac conjugated NKG2DL targeting agent), either alone or in combination with one or more additional therapeutic agents or treatment modalities.

### Additional Therapeutic Agents and Modalities

The treatment methods of the present disclosure, which include administration of a radioconjugated NKG2DL targeting agent, may further include administration of one or more additional therapeutic agents and/or treatment modalities. The additional agent and/or modality may be relevant (therapeutically effective) for the disease or condition being treated, when used alone or in combination or conjunction with a radioconjugated NKG2DL targeting agent. Such administration may be simultaneous, separate, or sequential with the administration of the effective amount of the radioconjugated NKG2DL targeting agent. For simultaneous administration, the agents may be administered as one composition if composition is possible or feasible, or as separate compositions.

Exemplary additional therapeutic agents and modalities that may be used include chemotherapeutic agents, small molecule anti-cancer agents, anti-inflammatory agents, immunosuppressive agents, immune-modulatory agents, endocrine agents, anti-androgens, external beam radiation, brachytherapy, HDAC inhibitors, LSD1 inhibitors, immune checkpoint therapies or blockades, DDR inhibitors, CD47 blockades, other radiolabeled cancer-targeting agents, adoptive cell therapy, and any combination thereof.

### A. Chemotherapeutic and Small Molecule Agents

Exemplary chemotherapeutic agents that may be used include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolomide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza®) and release of transcriptional repression (ATRA) therapies can also be combined with antibodies of the invention.

The chemotherapeutic agents include at least radiosensitizers, such as temozolomide, cisplatin, and/or fluorouracil.

The additional agents may, for example, include a bcl-2 inhibitor such as navitoclax or venetoclax (Venclexta®; Abbvie) and the combination may, for example, be used for the treatment of solid tumors such as breast cancer and lung cancer such as small cell lung carcinoma (SCLC).

The additional agents may, for example, include a cyclin-dependent kinase CDK4 and CDK6 inhibitor such as palbociclib (Ibrance®; Pfizer) and the combination may, for example, be used for the treatment of solid cancers such as breast cancers such as hormone receptor (HR)-positive, HER2-negative breast cancer, with or without an aromatase inhibitor.

The additional agents may, for example, include erlotinib (Tarceva®; Roche) and the combination may, for example, be used for the treatment of solid tumor cancers such as non-small cell lung cancer (NSCLC), for example, with mutations in the epidermal growth factor receptor (EGFR) and pancreatic cancer.

The additional agents may, for example, include sirolimus or everolimus (Affinitor®; Novartis) and the combination may, for example, be used for the treatment of solid tumor cancers such as melanoma and breast cancer.

The additional agents may, for example, include pemetrexed (Alimta®; Eli Lilly) and the combination may, for example, be used for the treatment of solid cancers such as mesothelioma such as pleural mesothelioma and lung cancer such as non-small cell lung cancer (NSCLC).

The chemotherapeutic or small molecule agents may, for example, be administered according to any standard dose regime known in the field. For example, chemotherapeutic agents may be administered at concentrations in the range of 1 to 500 mg/m², the amounts being calculated as a function of patient surface area (m²). For example, exemplary doses of the chemotherapeutic paclitaxel may include 15 mg/m² to 275 mg/m², exemplary doses of docetaxel may include 60 mg/m² to 100 mg/m², exemplary doses of epithilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the present disclosure.

B. External Beam Radiation and/or Brachytherapy

The additional therapeutic modality administered with the radioconjugated NKG2DL targeting agent, and optionally any other of the other additional therapeutics disclosed herein, may, for example, include an ionizing radiation, such as administered via external beam radiation or brachytherapy. Such radiation generally refers to the use of X-rays, gamma rays, or charged particles (e.g., protons or electrons) to generate ionizing radiation, such as delivered by a machine placed outside the patient's body (external-beam radiation therapy) or by a source placed inside a patient's body (internal radiation therapy or brachytherapy).

The external beam radiation or brachytherapy may enhance the targeted radiation damage delivered by the radioconjugated NKG2DL targeting agent and may thus be delivered sequentially with the radioconjugated NKG2DL targeting agent, such as before and/or after the radioconjugated NKG2DL targeting agent, or simultaneous with the radioconjugated NKG2DL targeting agents.

The external beam radiation or brachytherapy may be planned and administered in conjunction with imaging-based techniques such as computed tomography (CT) and/or magnetic resonance imaging (MRI) to accurately determine the dose and location of radiation to be administered. For example, a patient treated with any of the radioconjugated NKG2DL targeting agents disclosed herein may be imaged using either of CT or MRI to determine the dose and location of radiation to be administered by the external beam radiation or brachytherapy.

The radiation therapy may, for example, be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, and brachytherapy. The radiation therapy may be provided as a single dose or as fractionated doses, e.g., as 2 or more fractions. For example, the dose may be administered such that each fraction includes 2-20 Gy (e.g., a radiation dose of 50 Gy may be split up into 10 fractions, each including 5 Gy). The 2 or more fractions may be administered on consecutive or sequential days, such as once in 2 days, once in 3 days, once in 4 days, once in 5 days, once in 6 days, once in 7 days, or in a combination thereof.

### C. Immune Checkpoint Therapies

The additional agent(s) administered with the radioconjugated NKG2DL targeting agent may, for example, include an immune checkpoint therapy. Cancer cells have developed means to evade the standard checkpoints of the immune system. For example, cancer cells have been found to evade immunosurveillance through reduced expression of tumor antigens, downregulation of MHC class I and II molecules leading to reduced tumor antigen presentation, secretion of immunosuppressive cytokines such as TGFb, recruitment or induction of immunosuppressive cells such as regulatory T cells (Treg) or myeloid-derived suppressor cells (MDSC), and overexpression of certain ligands [e.g., programmed death ligand-1 (PD-L1)] that inhibit the host's existing antitumor immunity.

Another major mechanism of immune suppression by cancer cells is a process known as “T cell exhaustion”, which results from chronic exposure to tumor antigens, and is characterized by the upregulation of inhibitory receptors. These inhibitory receptors serve as immune checkpoints in order to prevent uncontrolled immune reactions.

Various immune checkpoints acting at different levels of T cell immunity have been described in the literature, including PD-1 (i.e., programmed cell death protein 1) and its ligands PD-L1 and PD-L2, CTLA-4 (i.e., cytotoxic T-lymphocyte associated protein-4) and its ligands CD80 and CD86, LAG3 (i.e., Lymphocyte-activation gene 3), B and T lymphocyte attenuator, TIGIT (T cell immunoreceptor with Ig and ITIM domains), TIM-3 (i.e., T cell immunoglobulin and mucin-domain containing protein 3), A2aR (Adenosine A2a Receptor), B7-H3 (B7 Homolog 3), B7-H4 (B7 Homolog 4), BTLA (B and T lymphocyte associated), VISTA (V-domain immunoglobulin suppressor of T cell activation), IDO (Indoleamine 2,3-Dioxygenase), TDO (Tryptophan 2,3-Dioxygenase), and KIR (Killer-Cell Immunoglobulin-Like Receptor).

Enhancing the efficacy of the immune system by therapeutic intervention is a particularly exciting development in cancer treatment. As indicated, checkpoint inhibitors such as CTLA-4 and PD-1 prevent autoimmunity and generally protect tissues from immune collateral damage. In addition, stimulatory checkpoints, such as OX40 (i.e., tumor necrosis factor receptor superfamily, member 4; TNFR-SF4), CD137 (i.e., TNFR-SF9), GITR (i.e., Glucocorticoid-Induced TNFR), CD27 (i.e., TNFR-SF7), CD40 (i.e., cluster of differentiation 40), and CD28, activate and/or promote the expansion of T cells.

Regulation of the immune system by inhibition of these proteins, such as by preventing binding between receptor and ligand, in combination with the radioconjugated NKG2DL targeting agents may provide synergistic therapeutic responses and enhanced therapeutic methods.

Thus, one aspect of the present invention provides the use of immune checkpoint therapies to remove certain blockades on the immune system that are utilized by cancer cells, in combination with the radioconjugated NKG2DL targeting agents disclosed herein. For example, antibodies against certain immune checkpoint inhibitors may be used to block interaction between checkpoint inhibitor proteins and their ligands, therefore preventing the signaling events that would otherwise have led to inhibition of an immune response against the tumor cell.

Moreover, there is a growing body of preclinical evidence supporting the ability of radiation to synergize with immune checkpoint inhibitor antibodies, and this is also being explored in the clinic with increasing numbers of clinical trials evaluating the combination of external beam radiation with immune checkpoint therapies across various tumor types and immune checkpoint inhibitor antibodies (Lamichhane, 2018). Clinical evidence supporting this combination has been generated in melanoma, with two studies demonstrating a clinical benefit using radiation in combination with the anti-cytotoxic T-lymphocyte-associated protein 4 (CTLA-4) immune checkpoint inhibitor antibody, ipilmumab (Twyman-Saint Vistor, 2015).

Accordingly, an object of the present disclosure is to provide therapies for the treatment of cancer using a radioconjugated NKG2DL targeting agent in combination with one or more immune checkpoint therapies, such as an inhibitor of an immune checkpoint protein.

Immune checkpoint therapies of the present disclosure include molecules that totally or partially reduce, inhibit, interfere with or modulate one or more checkpoint proteins, such as checkpoint proteins that regulate T cell activation or function. Immune checkpoint therapies may unblock an existing immune response inhibition by binding to or otherwise disabling checkpoint inhibition. The immune checkpoint therapy may include monoclonal antibodies, humanized antibodies, fully human antibodies, antibody fragments, peptides, small molecule therapeutics, or a combination thereof.

Exemplary immune checkpoint therapies include antibodies, peptides, and small molecules that may bind to and inhibit a checkpoint protein, such as the inhibitory receptors CTLA-4, PD-1, TIM-3, VISTA, BTLA, LAG-3, A2aR, and TIGIT. Additionally, the immune checkpoint therapies include antibodies, peptides, and small molecules that may bind to a ligand of any of the aforementioned checkpoint proteins, such as PD-L1, PD-L2, PD-L3, and PD-L4 (ligands for PD-1) and CD80 and CD86 (ligands for CTLA-4). Other exemplary immune checkpoint therapies may bind to checkpoint proteins such as the activating receptors CD28, OX40, CD40, GITR, CD137, CD27, and HVEM, or ligands thereof (e.g., CD137-L and GITR-L), CD226, B7-H3, B7-H4, BTLA, TIGIT, GALS, KIR, 2B4 (belongs to the CD2 family of molecules and is expressed on all NK, T6, and memory CD8+ (αβ) T cells), CD160 (also referred to as BY55), and CGEN-15049.

The CTLA-4 and PD-1 pathways are thought to operate at different stages of an immune response. CTLA-4 is considered the “leader” of the immune checkpoint inhibitors, as it stops potentially autoreactive T cells at the initial stage of naive T cell activation, typically in lymph nodes. The PD-1 pathway regulates previously activated T cells at the later stages of an immune response, primarily in peripheral tissues. Moreover, progressing cancer patients have been shown to lack upregulation of PD-L1 by either tumor cells or tumor-infiltrating immune cells. Immune checkpoint therapies targeting the PD-1 pathway might thus be especially effective in tumors where this immune suppressive axis is operational and reversing the balance towards an immune protective environment would rekindle and strengthen a pre-existing anti-tumor immune response. PD-1 blockade can be accomplished by a variety of mechanisms including antibodies that bind PD-1 or its ligand, PD-L1.

Accordingly, the immune checkpoint therapy may include an inhibitor of the PD-1 checkpoint, which may decrease, block, inhibit, abrogate, or interfere with signal transduction resulting from the interaction of PD-1 with one or more of its binding partners, such as PD-L1 and PD-L2. The inhibitor of the PD-1 checkpoint may, for example, be an anti-PD-1 antibody, antigen binding fragment, fusion proteins, oligopeptides, and other molecules that decrease, block, inhibit, abrogate or interfere with signal transduction resulting from the interaction of PD-1 with PD-L1 and/or PD-L2. The PD-1 checkpoint inhibitor may reduce the negative co-stimulatory signal mediated by or through cell surface proteins expressed on T lymphocytes so as render a dysfunctional T cell less dysfunctional (e.g., enhancing effector responses to antigen recognition). The PD-1 checkpoint therapy may be an anti-PD-1 antibody.

The immune checkpoint therapy may, for example, be an antibody against PD-1 such as nivolumab, or any of the inhibitors of PD-1 biological activity (or its ligands) disclosed in U.S. Pat. No. 7,029,674. Additional exemplary antibodies against PD-1 that may be used include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab); and AnaptysBio's anti-PD-1 antibody, known as ANBO11; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-O11), CureTech Ltd.

The immune checkpoint therapy may be an inhibitor of PD-L1 such as an antibody (e.g., an anti-PD-L1 antibody, i.e., ICI antibody), RNAi molecule (e.g., anti-PD-L1 RNAi), antisense molecule (e.g., an anti-PD-L1 antisense RNA), dominant negative protein (e.g., a dominant negative PD-L1 protein), and/or small molecule inhibitor. An exemplary anti-PD-L1 antibody includes clone EH12, or any of Genentech's MPDL3280A (RG7446); anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736 (Durvalumab).

The immune checkpoint therapy may, for example, be an inhibitor of PD-L2 or may reduce the interaction between PD-1 and PD-L2. Exemplary inhibitors of PD-L2 that may be used include antibodies (e.g., an anti-PD-L2 antibody, i.e., ICI antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

The immune checkpoint therapy may, for example, be an inhibitor of CTLA-4, such as an antibody against CTLA-4. An exemplary antibody that may be used against CTLA-4 includes ipilimumab. The anti-CTLA-4 antibody may block the binding of CTLA-4 to CD80 (B7-1) and/or CD86 (B7-2) expressed on antigen presenting cells. Exemplary antibodies against CTLA-4 that may be used further include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA-4 antibody clone BNI3 from Abcam. The immune checkpoint inhibitor may, for example, be a nucleic acid inhibitor of CTLA-4 expression.

The immune checkpoint therapy may, for example, be an inhibitor of LAG3. Lymphocyte activation gene-3 (LAG3) functions as an immune checkpoint in mediating peripheral T cell tolerance. LAG3 (also called CD223) is a transmembrane protein receptor expressed on activated CD4 and CD8 T cells, γδ T cells, natural killer T cells, B-cells, natural killer cells, plasmacytoid dendritic cells and regulatory T cells. The primary function of LAG3 is to attenuate the immune response. LAG3 binding to MHC class II molecules results in delivery of a negative signal to LAG3-expressing cells and down-regulates antigen-dependent CD4 and CD8 T cell responses. Thus, LAG3 negatively regulates the ability of T cells to proliferate, produce cytokines, and lyse target cells, termed as ‘exhaustion’ of T cells, and inhibition of LAG3 function may enhance T cell proliferation.

Monoclonal antibodies to LAG3 which may be used are known in the art and have been described, for example, in U.S. Pat. Nos. 5,976,877, 6,143,273, 6,197,524, 8,551,481, 10,898,571, and U.S. Pub. Nos. 20110070238, 20110150892, 20130095114, 20140093511, 20140127226, 20140286935, and in WO95/30750, WO97/03695, WO98/58059, WO2004/078928, WO2008/132601, WO2010/019570, WO2014/008218, EP0510079B1, EP0758383B1, EP0843557B1, EP0977856B1, EP1897548B2, EP2142210A1, and EP2320940B1. The anti-LAG3 checkpoint inhibitor antibody used may, for example, include relatlimab. Both of, such as combination of, relatlimab and a PD-1 inhibitor such as nivolumab may, for example, be used, such as Opdualag™ which includes relatlimab and nivolumab. Additionally, peptide inhibitors of LAG3 which may be used are also known and described in U.S. Pub. No. 20200369766.

The immune checkpoint therapy may be an inhibitor of the TIM3 protein. T-cell immunoglobulin and mucin-domain containing-3 (TIM3), also known as hepatitis A virus cellular receptor 2 (HAVCR2), is a type-I transmembrane protein that functions as a key regulator of immune responses. TIM3 has been shown to induce T cell death or exhaustion after binding to galectin-9, and to play an important in regulating the activities of many innate immune cells (e.g., macrophages, monocytes, dendritic cells, mast cells, and natural killer cells; Han, 2013). Like many inhibitory receptors (e.g., PD-1 and CTLA-4), TIM3 expression has been associated with many types of chronic diseases, including cancer. TIM3+ T cells have been detected in patients with advanced melanoma, non-small cell lung cancer, or follicular B-cell non-Hodgkin lymphoma. And the presence of TIM3+ regulatory T cells have been described as an effective indicator of lung cancer progression. Thus, inhibition of TIM3 may enhance the functions of innate immune cells. Exemplary TIM3 inhibitors include antibodies, peptides, and small molecules that bind to and inhibit TIM3.

The immune checkpoint therapy may be an inhibitor of the VISTA protein. The V-domain Ig suppressor of T cell activation (VISTA or PD-L3) is primarily expressed on hematopoietic cells, and its expression is highly regulated on myeloid antigen-presenting cells (APCs) and T cells. Expression of VISTA on antigen presenting cells (APCs) suppresses T cell responses by engaging its counter-receptor on T cells during cognate interactions between T cells and APCs. Inhibition of VISTA would enhance T cell-mediated immunity and anti-tumor immunity, suppressing tumor growth. In this regard, therapeutic intervention of the VISTA inhibitory pathway represents a novel approach to modulate T cell-mediated immunity, such as in combination with the presently disclosed radioconjugated NKG2DL targeting agents.

The immune checkpoint therapy may be an inhibitor of A2aR, or an A2aR blockade. The tumor microenvironment exhibits high concentrations of adenosine due to the contribution of immune and stromal cells, tissue disruption, and inflammation. A predominant driver is hypoxia due to the lack of perfusion that can lead to cellular stress and secretion of large amounts of ATP. Multiple small molecule inhibitors and antagonistic antibodies against these targets have been developed and show promising therapeutic efficacy against different solid tumors in clinical trials. For example, A2aR antagonists SYN115 and Istradefylline have been shown to improve motor function in patients with Parkinson's disease, and CPI-444 (NCT02655822, NCT03454451), PBF-509 (NCT02403193), NIR178 (NCT03207867), and AZD4635 (NCT02740985, NCT03381274) have been trialed for the treatment of various cancers. CPI-444 in combination with anti-PD-1 and anti-CTLA4 was highly effective in promoting CD8+ T cell responses and eliminating tumors in a preclinical. Additional exemplary A2aR inhibitors include, without limitation, the small molecule inhibitors SCH58261, ZM241365, and FSPTP.

The immune checkpoint therapy may include more than one modulator of an immune checkpoint protein. As such, the immune checkpoint therapy may include a first antibody or inhibitor against a first immune checkpoint protein and a second antibody or inhibitor against a second immune checkpoint protein.

### D. DNA Damage Response Inhibitors

The additional agent(s) administered with the radioconjugated NKG2DL targeting agent may, for example, include a DNA damage response inhibitor (DDRi). DNA damage can be due to endogenous factors, such as spontaneous or enzymatic reactions, chemical reactions, or errors in replication, or may be due to exogenous factors, such as UV or ionizing radiation or genotoxic chemicals. The repair pathways that overcome this damage are collectively referred to as the DNA damage response or DDR. This signaling network acts to detect and orchestrate a cell's response to certain forms of DNA damage, most notably double strand breaks and replication stress. Following treatment with many types of DNA damaging drugs and ionizing radiation, cells are reliant on the DDR for survival. It has been shown that disruption of the DDR can increase cancer cell sensitivity to these DNA damaging agents and thus may improve patient responses to such therapies.

Within the DDR, there are several DNA repair mechanisms, including base excision repair, nucleotide excision repair, mismatch repair, homologous recombinant repair, and non-homologous end joining. Approximately 450 human DDR genes code for proteins with roles in physiological processes. Dysregulation of DDR leads to a variety of disorders, including genetic, neurodegenerative, immune, cardiovascular, and metabolic diseases or disorders and cancers. For example, the genes OGG1 and XRCC1 are part of the base excision repair mechanism of DDR, and mutations in these genes are found in renal, breast, and lung cancers, while the genes BRCA1 and BRCA2 are involved in homologous recombination repair mechanisms and mutations in these genes leads to an increased risk of breast, ovarian, prostate, pancreatic, as well as gastrointestinal and hematological cancers, and melanoma. Exemplary DDR genes are provided in Table 1.

The methods disclosed herein may include administration of the radioconjugated NKG2DL targeting agents to deliver ionizing radiation in combination with a DDRi. Thus, the additional agent(s) administered with the radioconjugated NKG2DL targeting agent may target proteins in the DDR, i.e., DDR inhibitors or DDRi, thus maximizing DNA damage or inhibiting repair of the damage, such as in G1 and S-phase and/or preventing repair in G2, ensuring the maximum amount of DNA damage is taken into mitosis, leading to cell death.

TABLE 1

| | Gene | |
|---|---|---|
| DNA repair mechanism | examples | Cancer |
| Base Excision Repair | OGG1 | Renal, breast and lung cancer |
| | XRCC1 | Non-small cell lung cancer |
| Nucleotide Excision | ERCC1 | Lung and skin cancer, and glioma |
| Repair | XP | Xeroderma pigmentosum predisposing to skin cancer. Also |
| | | increased risk of bladder and lung cancer |
| Mismatch Repair | MSH2, | Lynch syndrome predisposing to colorectal cancer as well as |
| | MLH1 | endometrial, ovarian, stomach, small intestine, hepatobiliary tract, |
| | | upper urinary tract, brain and skin cancer |
| Homologous | BRCA1, | Increased risk of breast, ovarian, prostate, pancreatic, as well as |
| Recombinant Repair | BRCA2 | gastrointestinal and hematological cancer, and melanoma |
| Non-homologous End | KU70 | Breast, colorectal and lung cancer |
| Joining | KU80 | Lung cancer |
| Cell cycle checkpoints | ATM | Ataxia-telangiectasia predisposing to leukemia, breast and |
| | | pancreatic cancer |
| | ATR | Leukemia, lymphoma, gastric and endometrial cancer |

Moreover, one or more DDR pathways may be targeted to ensure cell death, i.e., lethality to the targeted cancer cells. For example, mutations in the BRCA1 and 2 genes alone may not be sufficient to ensure cell death, as other pathways, such as the PARP1 base excision pathway, may act to repair the DNA damage. Thus, combinations of multiple DDRi inhibitors or combining DDRi with antiangiogenic agents or immune checkpoint inhibitors, such as listed hereinabove, are possible and an object of the present disclosure.

### Exemplary DDRi—ATM and ATR Inhibitors

Ataxia telangiectasia mutated (ATM) and Ataxia talangiectasia mutated and Rad-3 related (ATR) are members of the phosphatidylinositol 3-kinase-related kinase (PIKK) family of serine/threonine protein kinases.

ATM is a serine/threonine protein kinase that is recruited and activated by DNA double-strand breaks. The ATM phosphorylates several key proteins that initiate activation of a DNA damage checkpoint, leading to cell cycle arrest, DNA repair, or cellular apoptosis. Several of these targets, including p53, CHK2, and H2AX, are tumor suppressors. The protein is named for the disorder ataxia telangiectasia caused by mutations of the ATM. The ATM belongs to the superfamily of phosphatidylinositol 3-kinase-related kinases (PIKKs), which includes six serine/threonine protein kinases that show a sequence similarity to a phosphatidylinositol 3-kinase (PI3K).

Like ATM, ATR is one of the central kinases involved in the DDR. ATR is activated by single stranded DNA structures, which may for example arise at resected DNA DSBs or stalled replication forks. When DNA polymerases stall during DNA replication, the replicative helicases continue to unwind the DNA ahead of the replication fork, leading to the generation of long stretches of single stranded DNA (ssDNA).

ATM has been found to assist cancer cells by providing resistance against chemotherapeutic agents and thus favors tumor growth and survival. Inhibition of ATM and/or ATR may markedly increase cancer cell sensitivity to DNA damaging agents, such as the ionizing radiation provided by the radioconjugated NKG2DL targeting agent. Accordingly, an object of the present disclosure includes administration of an inhibitor of ATM (ATMi) and/or ATR (ATRi), in combination with the NKG2DL targeting agents, to inhibit or kill cancer cells, such as those expressing tor overexpressing NKG2DL.

The inhibitor of ATM (ATMi) or ATR (ATRi) may be an antibody, peptide, or small molecule that targets ATM or ATR, respectively. Alternatively, an ATMi or ATRi may reduce or eliminate activation of ATM or ATR by one or more signaling molecules, proteins, or other compounds, or can result in the reduction or elimination of ATM or ATR activation by all signaling molecules, proteins, or other compounds. ATMi and/or ATRi also include compounds that inhibit their expression (e.g., compounds that inhibit ATM or ATR transcription or translation). An exemplary ATMi that may be used, KU-55933, suppresses cell proliferation and induces apoptosis. Other exemplary ATMi that may be used include at least KU-59403, wortmannin, CP466722, and KU-60019. Exemplary ATRi that may be used also include at least Schisandrin B, NU6027, NVP-BEA235, VE-821, VE-822, AZ20, and AZD6738.

Exemplary DDRi—Wee1 inhibitors

The checkpoint kinase Wee1 catalyzes an inhibitory phosphorylation of both CDK1 (CDC2) and CDK2 on tyrosine 15, thus arresting the cell cycle in response to extrinsically induced DNA damage. Deregulated Wee1 expression or activity is believed to be a hallmark of pathology in several types of cancer. For example, Wee1 is often overexpressed in glioblastomas, malignant melanoma, hepatocellular carcinoma, breast cancer, colon carcinoma, lung carcinoma, and head and neck squamous cell carcinoma. Advanced tumors with an increased level of genomic instability may require functional checkpoints to allow for repair of such lethal DNA damage. As such, the present inventors believe that Wee1 represents an attractive target in advanced tumors where its inhibition is believed to result in irreparable DNA damage. Accordingly, an object of the present disclosure includes administration of an inhibitor of Wee1, in combination with the NKG2DL targeting agents, to inhibit or kill cancer cells, such as those expressing or overexpressing NKG2DL.

A Wee1 inhibitor may be an antibody, peptide, or small molecule that targets Wee1. Alternatively, a Wee1 inhibitor may reduce or eliminate Wee1 activation by one or more signaling molecules, proteins, or other compounds, or can result in the reduction or elimination of Wee1 activation by all signaling molecules, proteins, or other compounds. The term also includes compounds that decrease or eliminate the activation or deactivation of one or more proteins or cell signaling components by Wee1 (e.g., a Wee1 inhibitor can decrease or eliminate Wee1-dependent inactivation of cyclin and Cdk activity). Wee1 inhibitors also include compounds that inhibit Wee1 expression (e.g., compounds that inhibit Wee1 transcription or translation).

Exemplary Wee1 inhibitors that may be used include AZD-1775 (adavosertib), and inhibitors such as those described in any of, e.g., U.S. Pat. Nos. 7,834,019; 7,935,708; 8,288,396; 8,436,004; 8,710,065; 8,716,297; 8,791,125; 8,796,289; 9,051,327; 9,181,239; 9,714,244; 9,718,821; and 9,850,247; U.S. Pat. App. Pub. Nos. US 2010/0113445 and 2016/0222459; and Intl Pat. App. Pub. Nos. WO2002/090360, WO2015/019037, WO2017/013436, WO2017/216559, WO2018/011569, and WO2018/011570.

Further Wee1 inhibitors that may be used include a pyrazolopyrimidine derivative, a pyridopyrimidine, 4-(2-chlorophenyl)-9-hydroxypyrrolo[3,4-c]carbazole-1,3-(2H, 6H)-dione (CAS No. 622855-37-2), 6-butyl-4-(2-chlorophenyl)-9-hydroxypyrrolo[3,4-c]carbazole-1,3-(2H,6H)-dione (CAS No. 62285550-9), 4-(2-phenyl)-9-hydroxypyrrolo[3,4-c]carbazole-1,3-(2H,6H)-dione (CAS No. 1177150-89-8), and an anti-Wee1 small interfering RNA (siRNA) molecule.

### Exemplary DDRi-PARP Inhibitors

Another exemplary DDRi of the present disclosure is an inhibitor of poly(ADP-ribose) polymerase (“PARP”). Inhibitors of the DNA repair protein PARP, referred to individually and collectively as “PARPi”, have been approved for use in a range of solid tumors, such as breast and ovarian cancer, particularly in patients having BRCA1/2 mutations. BRCA1 and 2 function in homologous recombination repair (HRR). When mutated, they induce genomic instability by shifting the DNA repair process from conservative and precise HRR to non-fidelitous methods such as DNA endjoining, which can produce mutations via deletions and insertions.

PARPi have been shown to exhibit synthetic lethality, as exhibited by potent single agent activity, in BRCA1/2 mutant cells. This essentially blocks repair of single-strand DNA breaks. Since HRR is not functional in these tumor cells, cell death results. Because most tumors do not carry BRCA1 or BRCA2 mutations, the potency of PARPi in such tumors is far less pronounced.

To date, the FDA has approved four PARPi drugs (olaparib, niraparib, rucaparib and talazoparib) as monotherapy agents, specifically in patients with germline and somatic mutations in the BRCA1 and BRCA2 genes. Along with veliparib, olaparib, niraparib and rucaparib were among the first generation of PARPi that entered clinical trials. Their IC50 values were found to be in the nanomolar range. In contrast, second generation PARPi like talazoparib have IC50 values in the picomolar range.

These PARPi all bind to the binding site of the cofactor, b nicotinamide adenine dinucleotide (b-NAD+), in the catalytic domain of PARPi and PARP2. The PARP family of enzymes use NAD+ to covalently add Poly(ADP-ribose) (PAR) chains onto target proteins, a process termed “PARylation.” PARP1 (which is the best-studied member) and PARP2, are important components of the DNA damage response (DDR) pathway. PARP1 is involved in the repair of single-stranded DNA breaks, and possibly other DNA lesions (Woodhouse, et al.; Krishnakumar, et al.). Through its zinc finger domains, PARP1 binds to damaged DNA and then PARylates a series of DNA repair effector proteins, releasing nicotinamide as a by-product (Krishnakumar, et al.). Subsequently, PARP1 auto-PARylation leads to release of the protein from the DNA. The available PARPi, however, differ in their capability to trap PARP1 on DNA, which seems to correlate with cytotoxicity and drug efficacy. Specifically, drugs like talazoparib and olaparib are more effective in trapping PARP1 than are veliparib (Murai, et al., 2012; Murai, et al., 2014).

The efficacy of PARPi in ovarian cancer and breast cancer patients who have loss-of-function mutations in BRCA1 or BRCA2 genes is largely attributed to the genetic concept of synthetic lethality: that proteins of BRCA 1 and 2 normally maintain the integrity of the genome by mediating a DNA repair process, known as homologous recombination repair (HRR); and PARPi causes a persistent DNA lesion that, normally, would otherwise be repaired by HR. In the presence of PARPi, PARP1 is trapped on DNA which stalls progression of the replication fork. This stalling is cytotoxic unless timely repaired by the HR system. In cells lacking effective HR, they are unable to effectively repair these DNA lesions, and thus die.

Again, mutations in BRCA genes and others in the HRR system are not prevalent in many cancer types. So, to better harness the therapeutic benefits of PARPi in such cancers, one can induce “artificial” synthetic lethality by pairing a PARPi with either chemotherapy or radiation therapy. Preclinical studies have demonstrated that combining radiation therapy and PARPi can increase the sensitivity of BRCA1/2 mutant tumor cells to PARP inhibition and extend the sensitivity of non-mutant BRCA tumors to PARP inhibition. Additional studies have shown that ionizing radiation (TR) itself can mediate PARPi synthetic lethality in tumor cells.

Accordingly, the presently disclosed methods include administration of the radioconjugated NKG2DL targeting agents that deliver ionizing radiation in combination with a PARPi.

Exemplary PARPi agents that may be used include any known agent performing that function, such as any of those approved by the FDA. For example, the PARPi used may include olaparib (Lynparza®), niraparib (Zejula®), rucaparib (Rubraca®) and/or talazoparib (Talzenna®). The present inventors realized that the effect of the PARPi may be improved through increases in dsDNA breaks induced by ionizing radiation provided by the radioconjugated NKG2DL targeting agent while these repair pathways are being blocked by the PARPi.

### E. CD47 Blockades

An additional agent administered with the radioconjugated NKG2DL targeting agent may be a CD47 blockade, such as any agent that interferes with, or reduces the activity and/or signaling between CD47 (e.g., on a target cell) and SIRPα (e.g., on a phagocytic cell), for example, through interaction with either CD47 or SIRPα. Non-limiting examples of suitable CD47 blockades include CD47 and/or SIRPα reagents, including without limitation SIRPα polypeptides, anti-SIRPα antibodies, soluble CD47 polypeptides, and anti-CD47 antibodies or antibody fragments.

As used herein, the term “CD47 blockade” refers to any agent that reduces the binding of CD47 (e.g., on a target cell) to SIRPα (e.g., on a phagocytic cell) or otherwise downregulates the “don't eat me” signal of the CD47-SIRPα pathway. Non-limiting examples of suitable anti-CD47 blockades include SIRPα reagents, including without limitation SIRPα polypeptides, anti-SIRPα antibodies, soluble CD47 polypeptides, and anti-CD47 antibodies or antibody fragments. According to certain aspects, a suitable anti-CD47 agent (e.g. an anti-CD47 antibody, a SIRPα reagent, etc.) specifically binds CD47 to reduce the binding of CD47 to SIRPα.

A CD47 blockade agent for use in the methods of the invention may, for example, up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to phagocytosis in the absence of the agent. Similarly, an in vitro assay for levels of tyrosine phosphorylation of SIRPα may, for example, show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the agent.

According to certain aspects, a SIRPα reagent may include the portion of SIRPα that is sufficient to bind CD47 at a recognizable affinity, which normally lies between the signal sequence and the transmembrane domain, or a fragment thereof that retains the binding activity. Accordingly, suitable CD47 blockades that may be employed include any of the SIRPα-IgG Fc fusion proteins and others disclosed in U.S. Pat. No. 9,969,789 including without limitation the SIRPα-IgG Fc fusion proteins TTI-621 and TTI-622 (Trillium Therapeutics, Inc.), both of which preferentially bind CD47 on tumor cells while also engaging activating Fc receptors. A SIRPα-IgG Fc fusion protein including the amino acid sequence SEQ ID NO:52, SEQ ID NO:53, or SEQ ID NO:54 may, for example, be used. Still other SIRPα Fc domain fusions proteins that may be used include ALX148 from Alx Oncology or any of those disclosed in Int'l Pub. No WO2017027422 or U.S. Pat. No. 10,696,730.

According to certain aspects, an anti-CD47 agent includes an antibody that specifically binds CD47 (i.e., an anti-CD47 antibody) and reduces the interaction between CD47 on one cell (e.g., an infected cell) and SIRPα on another cell (e.g., a phagocytic cell). Non-limiting examples of suitable antibodies include clones B6H12, 5F9, 8B6, and C3 (for example as described in International Pub. No. WO2011/143624). Suitable anti-CD47 antibodies include fully human, humanized or chimeric versions of such antibodies.

Exemplary human or humanized antibodies useful for in vivo applications in humans due to their low antigenicity include at least monoclonal antibodies against CD47, such as Hu5F9-G4, a humanized monoclonal antibody available from Gilead as Magrolimab (Sikic, et al. (2019) Journal of Clinical Oncology 37:946); Lemzoparlimab and TJC4 from I-Mab Biopharma; AO-176 from Arch Oncology, Inc; AK117 from Akesobio Australia Pty; IMC-002 from Innovent Biologics; ZL-1201 from Zia Lab; SHR-1603 from Jiangsu HengRui Medicine Co.; and SRF231 from Surface Oncology. Bispecific monoclonal antibodies are also available, such as IBI-322, targeting both CD47 and PD-L1 from Innovent Biologics.

AO-176, in addition to inducing tumor phagocytosis through blocking the CD47-SIRPα interaction, has been found to preferentially bind tumor cells versus normal cells (particularly RBCs where binding is negligible) and directly kills tumor versus normal cells.

Antibodies against SIRPα may also be used as CD47 blockades. Without limitation, anti-SIRPα antibodies (also referred to as SIRPα antibodies herein) that may be used in or embodied in any of the aspects of the invention include but are not limited to the following anti-SIRPα antibodies, antibodies that include one or both of the heavy chain and light chain variable regions of the following anti-SIRPα antibodies, antibodies that include one or both of the heavy chain and the light chain CDRs of any of the following anti-SIRPα antibodies, and antigen-binding fragments of any of said anti-SIRPα antibodies:
(1) ADU-1805 (Sairopa B. V.; Aduro) and any of the SIRPα antibodies disclosed in Intl. Pub. No. WO2018190719 or U.S. Pat. No. 10,851,164;
(2) AL008 (Alector LLC) and any of the SIRPα antibodies disclosed in Intl. Pub. No. WO2018107058, U.S. Pub. No. 20190275150, or U.S. Pub. No. 20210179728;
(3) AL008 (Apexigen, Inc.) and any of the SIRPα antibodies disclosed in Intl. Pub. No. WO2021174127 or U.S. App. No. 63/108,547;
(4) SIRP-1 and SIRP-2 (Arch Oncology, Inc.) and any of the SIRPα antibodies disclosed in Intl. Pub. No. WO2021222746, U.S. App. No. 63/107,200 or U.S. Pub. No. 20200297842;
(5) OSE-172 (a/k/a BI 765063; Boehringer Ingelheim) and any of the SIRPα antibodies disclosed in Intl. Pub. No. WO2017178653 or U.S. Pub. No. 20190127477;
(6) CC-95251 (Bristol Myers Squibb; Celgene) and any of the SIRPα antibodies disclosed in Intl. Pub. No. WO2020068752 or U.S. Pub. No. 20200102387;
(7) ES004 (Elpiscience Biopharma) and any of the SIRPα antibodies disclosed in Intl. Pub. No. WO2021032078 or U.S. Pub. No. 20210347908;
(8) FSI-189 (Gilead Sciences, Inc.; Forty Seven) and any of the SIRPα antibodies disclosed in Intl. Pub. No. WO2019023347, U.S. Pat. No. 10,961,318 or U.S. Pub. No. 20210171654;
(9) BYON4228 (Byondis B. V.; Synthon) and any of the SIRPα antibodies disclosed in Intl. Pub. No. WO2018210793, Intl. Pub. No. WO2018210795, or U.S. Pub. No. 20210070874;
(10) any of the SIRPα antibodies disclosed in Intl. Pub. No. WO2018057669, U.S. Pat. No. 11,242,404 or U.S. Pub. No. 20220002434 (Alexo Therapeutics Inc., now ALX Oncology Inc.);
(11) any of the SIRPα antibodies disclosed in Intl. Pub. No. WO2015138600, U.S. Pat. No. 10,781,256 or U.S. Pat. No. 10,081,680 (Leland Stanford Junior University);
(12) BR105 (Bioray Pharma); or
(13) BSI-050 (Biosion, Inc.).

The CD47 blockade may alternatively, or additionally, include agents that modulate the expression of CD47 and/or SIRPα, such as phosphorodiamidate morpholino oligomers (PMO) that block translation of CD47 such as MBT-001 (PMO, morpholino, Sequence: 5′-CGTCACAGGCAGGACCCACTGCCCA-3′) [SEQ ID NO:55]) from Morphiex or any of the PMO oligomer CD47 inhibitors disclosed in any of U.S. Pat. Nos. 8,557,788, 8,236,313, 10,370,439 and Int'l Pub. No. WO2008060785.

Small molecule inhibitors of the CD47-SIRPα axis may also be used, such as RRx-001 (1-bromoacetyl-3,3 dinitroazetidine) from EpicentRx and Azelnidipine (CAS number 123524-52-7), or pharmaceutically acceptable salts thereof. Such small molecule CD47 blockades may, for example, be administered at a dose of 5-100 mg/m², 5-50 mg/m², 5-25 mg/m², 10-25 mg/m², or 10-20 mg/m², or in any of the dose ranges or at any of the doses described herein. Administration of RRx-001 may, for example, be once or twice weekly and be by intravenous infusion. The duration of administration may, for example, be at least one, two, three or four weeks.

Various CD47 blockades that may be used are found in Table 1 of Zhang, et al., (2020), Frontiers in Immunology vol 11, article 18, and in Table 2 below.

TABLE 2

| Company | Approach | Agent/Program |
|---|---|---|
| Akesobio Australia Pty Ltd | CD47 mAb | AK117 |
| Arch Oncology (Tioma Therapeutics) | CD47 mAb | AO-176 |
| Elpiscience Biopharma Inc. | CD47 | ES004 |
| EpicentRx | Small molecule inhibitor | RRx-001 |
| | of dinitroazetidine | (1-bromoacetyl-3,3 |
| | hypoxia sensor to | dinitroazetidine) |
| | downregulate | |
| | CD47/SIRPα | |
| ImmuneOncia Therapeutics | CD47 mAb human | IMC-002 |
| Innovent Biologics | CD47 mAb | IBI-188 (CD47 mAb) |
| | CD47/PD-L1 bispecific | IBI-322 (Bispecific) |
| | mAb | |
| OSE | SIRPα mAb | BI 765063 (OSE-172) |
| Zai Lab | CD47 mAb | ZL-1201 |
| Alx Oncology | High-affinity SIRPα-Fc | ALX148 |
| Gilead/Forty Seven | CD47 mAb | Magrolimab |
| | SIRPα mAb | FSI-189 |
| I-Mab Biopharma | CD47 mAb | TJC4 |
| Jiangsu HengRui Medicine Co., Ltd. | CD47 mAb | SHR-1603 |
| Surface Oncology | CD47 mAb human | SRF231 |
| Morphiex | CD47 targeting | MBT-001 |
| | phosphorodiamidate | |
| | morpholino oligomers | |

Therapeutically effective doses of an anti-CD47 antibody or other protein CD47 blockade may, for example, be a dose that leads to sustained serum levels of the protein of about μg/ml or more (e.g., about 50 ug/ml or more, about 60 ug/ml or more, about 75 ug/ml or more, about 100 ug/ml or more, about 125 ug/ml or more, or about 150 ug/ml or more). Therapeutically effective doses or administration of a CD47 blockade, such as an anti-CD47 antibody or SIRPα fusion protein or small molecule, include, for example, amounts of 0.05-10 mg/kg (agent weight/subject weight), such as at least 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg; or not more than 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, 2.0 mg/kg, 1.5 mg/kg, 1.0 mg/kg, or any combination of these upper and lower limits. Therapeutically effective doses of a small molecule CD47 blockade such as those disclosed herein also, for example, include 0.01 mg/kg to 1,000 mg/kg and any subrange or value of mg/kg therein such as 0.01 mg/kg to 500 mg/kg or 0.05 mg/kg to 500 mg/kg, or 0.5 mg/kg to 200 mg/kg, or 0.5 mg/kg to 150 mg/kg, or 1.0 mg/kg to 100 mg/kg, or 10 mg/kg to 50 mg/kg.

According to certain aspects, the anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example, as described in U.S. Pub. No. 20100239579).

### F. Epigenetic Agents

The additional therapy or modality administered in combination with or in conjunction with the radioconjugated NKG2DL targeting agent may, for example, include one or more epigenetic agents, such as inhibitors of one or both of a Histone deacetylase (HDAC) and a Lysine-specific histone demethylase 1A (LSD1) also known as lysine (K)-specific demethylase 1A (KDM1A). The HDAC inhibitor may, for example, be an inhibitor of class I HDACs, i.e., an HDAC1 inhibitor.

HDAC inhibitor drugs that may be used include, for example, vorinostat (e.g., Zolinza®), romidepsin (e.g., Istodax®), belinostat (e.g., Beleodaq®), and panobinostat (e.g., Farydak®), Valproic acid (Depacon®) or Entinostat, or any combination thereof. Any of these HDAC inhibitors or pharmaceutically acceptable salts thereof may be used individually or in any combination in the various aspects of the present invention. Vorinostat and romidepsin are FDA-approved for treating patients having cutaneous T-cell lymphoma. Belinostat is FDA-approved for treating patients having peripheral T-cell lymphoma. Panobinostat is FDA-approved for treating patients having multiple myeloma. Vorinostat may, for example be orally dosed at 100-1,000 mg per day such as 400 mg per day. Romidepsin may, for example, be intravenously dosed at 10-30 mg/m2 such as 14 mg/m2. Belinostat may, for example, be intravenously dosed at 500-1,500 mg/m2 such as 1,000 mg/m2. Panobinostat may, for example, be orally dosed at 5-50 mg per day, such as 20 mg per day. Valproic acid may, for example, be orally dosed at 10-400 mg/kg such as 20-300 mg/kg. Entinostat may, for example, be orally dosed at 2-50 mg per day, such as 4-30 mg per day such as 15-30 mg per day, or 20 mg per day.

LSD1 inhibitors that may be used in the various aspects of the invention include, for example, TCP (tranylcypromine), ORY-1001 (iadademstat), GSK2879552 (GSK), INCB059872, IMG-7289 (bomedemstat), ORY-2001 (vafidemstat), CC-90011, seclidemstat, or a pharmaceutically acceptable salt of any of the preceding, or any combination thereof.

Administration of these epigenetic agents may, for example, be every day, every other day, every three days, every four days, or weekly.

### G. Adoptive Cell Therapy

The additional therapy or modality that may be administered in combination with or in conjunction with the radioconjugated NKG2DL targeting agent may, for example, include an adoptive cell therapy (ACT). ACT is the transfer of ex vivo grown and/or modified cells, most commonly immune-derived cells, into a host with the goal of transferring the immunologic functionality and characteristics of the transplanted cells.

The ACT may, for example, include a population of cells (e.g., T cells, NK cells, or dendritic cells) expressing a CAR or TCR (referred herein simply as “CAR cell therapy”) or complexed with an exogenous targeting agent to target the ACT cells to cells expressing a preselected antigen, such as a cancer associated antigen, e.g., as described in U.S. Pub. No. 20210169936. A CAR cell therapy may, for example, involve engineering a T cell, NK cell, or dendritic cell to target a tumor antigen of interest by way of engineering a desired antigen binding domain that specifically binds to an antigen on a tumor cell. In the context of the present invention, “tumor antigen” or “proliferative disorder antigen” or “antigen associated with a proliferative disorder,” refers to antigens that are common to specific proliferative disorders such as cancer.

The ACT administered in combination with or in conjunction with or used with a radiolabeled NKG2DL targeting agent may, for example, include one of more of the following FDA-approved ACT products: Abecma® (idecabtagene vicleucel; Celgene Corporation, a Bristol-Myers Squibb Company), targeted to BCMA, for example, for the treatment of multiple myeloma, such as relapsed and/or refractory multiple myeloma; Breyanzi® (lisocabtagene maraleucel; Juno Therapeutics, Inc., a Bristol-Myers Squibb Company), targeted to CD19, for example, for the treatment of non-Hodkin lymphoma, NHL such as large B cell NHL; Carvykti® (ciltacabtagene autoleucel; Janssen Biotech, Inc.), targeted to BCMA, for example, for the treatment of multiple myeloma, such as relapsed and/or refractory multiple myeloma; Kymriah® (tisagenlecleucel; Novartis Pharmaceuticals Corp.) targeted to CD19, for example, for the treatment of lymphomas such as large B-cell lymphoma (DLBCL); Tecartus® (brexucabtagene autoleucel; Kite Pharma, Inc.) targeted to CD19, for example, for the treatment of lymphomas such as mantle cell lymphoma and acute lymphoblastic leukemia (ALL) such as B-cell precursor ALL; and Yescarta® (axicabtagene ciloleucel; Kite Pharma, Inc.) targeted to CD19, for example, for the treatment of lymphomas such as large B-cell lymphoma (DLBCL).

A number of tumor-specific and tumor-associated antigens have been catalogued and are maintained in databases, such as the Database of Collected Peptides for Neoantigen (biostatistics.online/dbPepNeo/), Tumor-Specific NeoAntigen database (biopharm.zju.edu.cn/tsnadb/), TANTIGEN 2.0: Tumor T-cell Antigen Database (projects.met-hilab.org/tadb/), and Cancer Antigenic Peptide Database (caped.icp.ucl.ac.be/). Tumor antigens listed in these databases, as well as additional antigens identified in patients, may for example be used as ACT targets in aspects of the present invention that include ACT.

In aspects of the invention involving CAR/TCR expressing cell ACT or other antigen-targeting ACT, exemplary antigen targets may include CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRvIII, ROR1, mesothelin, CD33/IL3Ra, c-Met, CD37, PSMA, Glycolipid F77, GD-2, gp100, NY-ESO-1 TCR, FRalpha, GUCY2C, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or any combination thereof (e.g., both CD33 and CD123).

An exemplary total dose for ACT may, for example, include, 10³ to 10¹¹ cells/kg body weight of the subject, such as 10³ to 10¹⁰ cells/kg body weight, or 10³ to 10⁹ cells/kg body weight of the subject, or 10³ to 10⁸ cells/kg body weight of the subject, or 10³ to 10⁷ cells/kg body weight of the subject, or 10³ to 10⁶ cells/kg body weight of the subject, or 10³ to 10⁵ cells/kg body weight of the subject. Moreover, an exemplary total dose includes 10⁴ to 10¹¹ cells/kg body weight of the subject, such as 10⁵ to 10¹¹ cells/kg body weight, or 10⁶ to 10¹¹ cells/kg body weight of the subject, or 10⁷ to 10¹¹ cells/kg body weight of the subject.

An exemplary total dose may be administered based on a patient body surface area rather than the body weight. As such, the total dose may include 10³ to 10¹³ cells per m².

The radioconjugated NKG2DL targeting agent may, for example, be administered after administration of the effective dose of the ACT or CAR cell therapy. The effective amount of the radioconjugated NKG2DL targeting agent may be an amount sufficient to induce depletion or ablation of NKG2DL expressing malignant cells in the subject.

Accordingly, in one aspect the present invention provides methods for the treatment of a proliferative disease, such as a solid cancer, which include administration of a radioconjugated NKG2DL targeting agent and an adoptive cell therapy. The adoptive cell therapy may, for example, include apheresis of autologous cells which may be gene edited prior to reinfusion (adoptive cell therapy such as CAR T-cell therapy), such as after administration of the radioconjugated NKG2DL targeting agent, or simultaneous with administration of the radioconjugated NKG2DL targeting agent.

Thus, in one aspect the present invention provides a method for treating a mammalian subject, such as a human, afflicted with cancer or precancerous proliferative disorder, such as a solid tumor or a hematological malignancy, including (i) administering to the subject an amount of a radioconjugated NKG2DL targeting agent, and (ii) either before, simultaneous or overlapping with, or after (such as after a suitable time period), performing adoptive cell therapy on the subject to treat the subject's cancer.

Although the invention provides certain aspects that involve adoptive cell therapy and/or the administration of cells therefor to a subject, also provided are aspects of the invention or variations of the non-ACT aspects of the invention that do not involve or include cell therapy or the administration of cells to the subject, such as do not involve or include the administration of genetically edited cells to the subject, and/or do not involve the administration of CAR-T or recombinant TCR cells to the subject and/or do not involve the administration of NK cells to the subject.

### H. Other Targeting Agents

Additional agents used in combination with or conjunction with a radiolabeled NKG2DL targeting agent may, for example, include agents targeting other cancer-associated antigens such as any of those disclosed herein. Such cancer-associated antigens may for example be expressed by/on cancer cell themselves or by immune suppressive cells such as MDSCs and Treg cells. Exemplary cancer-associated antigen targets for such targeting agents include CD33, CD38, DR5, 5T4, HER2, HER3, TROP2, or any of those disclosed herein. Such targeting agents for use in the various aspects of the present invention may be radiolabeled, drug-conjugated, or naked (if therapeutically active).

Exemplary DR5 targeting agents that may be used, for example, as a radioconjugate, include any one or more of the monoclonal anti-DR5 antibodies mapatumumab, conatumumab, lexatumumab, tigatuzumab, drozitumab, and LBY-135.

Exemplary 5T4 targeting agents that may be used, for example, as a radioconjugate, include any one or more of the monoclonal anti-5T4 antibodies MED10641, ALG.APV-527, Tb535, H6-DM5, and ZV0508.

Exemplary HER2 targeting agents that may be used, for example, as a radioconjugate, include the monoclonal antibodies trastuzumab and pertuzumab. The amino acid sequences of the light chain and the heavy chain of Trastuzumab reported by DrugBank Online are: light chain (SEQ ID NO:86) and heavy chain (SEQ ID NO:87). The amino acid sequences of the light chain and the heavy chain of Pertuzumab reported by DrugBank Online are: light chain (SEQ ID NO:88) and heavy chain (SEQ ID NO:89). The HER2 targeting agent used may, for example, be the ADC fam-trastuzumab deruxtecan-nxki (Enhertu®).

Exemplary HER3 targeting agents that may be used for example as a radioconjugate, include any one or more of the monoclonal antibodies patritumab, seribantumab, lumretuzumab, elgemtumab, GSK2849330, or AV-203 (Aveo Oncology) or any of the anti-HER3 antibodies disclosed in U.S. Pat. Nos. 10,494,441; 9,828,635 or U.S. Pub. No. 20210025006. An exemplary HER3 antibody includes an immunoglobulin heavy chain variable region including a CDRH1 including SEQ ID NO:56, a CDRH2 including SEQ ID NO:57, and a CDRH3 including SEQ ID NO:58, an immunoglobulin light chain variable region including a CDRL1 including SEQ ID NO:59, a CDRL2 including SEQ ID NO:60, and a CDRL3 including SEQ ID NO:61. An exemplary HER3 antibody includes an immunoglobulin heavy chain variable region including SEQ ID NO:62 and/or an immunoglobulin light chain variable region including SEQ ID NO:63. An exemplary HER3 antibody includes an immunoglobulin heavy chain amino acid sequence of SEQ ID NO:64 and/or an immunoglobulin light chain amino acid sequence of SEQ ID NO:65. The HER3 targeting agent used may, for example, be the ADC patritumab deruxtecan.

Exemplary TROP2 targeting agents that may be used, for example, as a radioconjugate, in conjunction with a radiolabeled NKG2DL targeting agent in the treatment of a proliferative disorder include the monoclonal antibodies Sacituzumab and Datopotamab, antibodies having one or both of the heavy chain and light chain of said antibodies, and antibodies having one or both of the heavy chain CDRs and the light chain CDRs of said antibodies, or TROP2-binding fragments of any of the aforementioned antibodies. Sacituzumab biosimilar is commercially available as Catalog No. A2175 from BioVision Incorporated (an Abcam company, Waltham, MA, USA). Datopotamab biosimilar is commercially available as Catalog No. PX-TA1653 from ProteoGenix (Schiltigheim, France). The TROP2 targeting agent used may, for example, be the ADC sacituzumab govitecan-hziy (Trodelvy®).

Exemplary TROP2 targeting agents that may be used, for example as a radioconjugate, in combination or conjunction with a radiolabeled NKG2DL targeting agent in the treatment of a proliferative disorder include a monoclonal antibody having a heavy chain SEQ ID NO:66 and/or a light chain SEQ ID NO:71 (reported as the heavy and light chains of Sacituzumab), or an antibody including one or both of the heavy chain variable region (SEQ ID NO:67) or the light chain variable region (SEQ ID NO:72) of said chains, or an antibody including 1, 2, or 3 of the heavy chain CDRs of said heavy chain (CDR H1-3: SEQ ID NOS:68-70 respectively) and/or 1, 2 or 3 of the light chain CDRs of said light chain (CDR L1-3: SEQ ID NOS:73-75 respectively), and any of the anti-human TROP antibodies disclosed in U.S. Pat. No. 7,238,785 (hRS7), U.S. Pat. No. 9,492,566, 10,195,517, or 11,116,846, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

Further exemplary TROP2 targeting agents that may be radiolabeled and used in conjunction with a radiolabeled NKG2DL targeting agent in the treatment of a proliferative disorder include a monoclonal antibody heavy chain SEQ ID NO:76 and/or a light chain SEQ ID NO:81 (reported as the heavy and light chains of Datopotamab), or an antibody including one or both of the variable region of said heavy chain (SEQ ID NO:77) and the variable region of said light chain (SEQ ID NO:82), or an antibody including 1, 2, or 3 of the heavy chain CDRs of said heavy chain (CDRs 1-3: SEQ ID NOS:78-80 respectively) and/or 1, 2 or 3 of the light chain CDRs of the said light chain (CDR H1-3: SEQ ID NOS:83-85 respectively), and any of the anti-human TROP antibodies disclosed in Int'l Pub. No. WO2015098099 or U.S. Pub. No. 20210238303, or an antibody including one or both of the heavy chain and light chain variable regions of said antibodies, or an antibody including a heavy chain including 1, 2 or 3 of the heavy chain CDRs of any of said antibodies and/or a light chain including 1, 2, or 3 of the light chain CDRs of any of said antibodies.

Exemplary CD33 targeting agents that may be radiolabeled, drug-conjugated, or unlabeled for use in combination or conjunction with a radiolabeled NKG2DL targeting agent include the monoclonal antibodies lintuzumab, gemtuzumab, and vadastuximab. In combination with a radiolabeled NKG2DL targeting agent as disclosed herein, a CD33 targeting therapeutic agent may, for example, be used to treat hematological cancers such as AML, MDS, CML, MM or any of those disclosed herein. In combination with a radiolabeled NKG2DL targeting agent as disclosed herein, a CD33 targeting therapeutic agent may, for example, be used to treat solid cancers, such as ovarian, breast, cervical prostate, osteosarcoma, gastric, bladder, lung, melanoma, colorectal and squamous cell carcinoma cancers and any of the cancers disclosed herein, for example, by depleting myeloid-derived suppressor cells (MDSCs). In one aspect, the CD33 targeting agent used in combination with a radiolabeled NKG2DL targeting agent is ²²⁵Ac-lintuzumab. In another aspect, the CD33 targeting agent used in combination with a radiolabeled HER3 targeting agent is the ADC gemtuzumab ozogamicin (Mylotarg®; Pfizer).

Exemplary CD38 targeting agents that may be radiolabeled, drug-conjugated, or unlabeled for use in combination or conjunction with a radiolabeled NKG2DL targeting agent include anti-CD38 monoclonal antibodies such as daratumumab (Darzalex®; Johnson and Johnson) and isatuximab (Sarclisa®; Sanofi) or antigen-binding fragments thereof. Such CD38 targeting agents may, for example, be used in combination with the radiolabeled CCR8 targeting agent(s) in the treatment of solid tumors that may, for example, be infiltrated with CD38-positive suppressive immune cells, such as but not limited to ovarian, breast, cervical prostate, gastric, bladder, lung, melanoma, colorectal and squamous cell carcinoma cancers and any of the cancers disclosed herein.

In one aspect of the invention a radiolabeled targeting agent used in combination or conjunction with a NKG2DL targeting agent may be a radiolabeled PSMA-targeting agent such as a radiolabeled anti-PSMA monoclonal antibody such as J591 labeled for example with ¹⁷⁷Lu or ²²⁵Ac or Rosopatamab labeled for example with ¹⁷⁷Lu or ²²⁵Ac, or a radiolabeled PSMA-binding small molecule such as PSMA-617 labeled for example with ¹⁷⁷Lu (such as Pluvicta®, Novartis) or ²²⁵Ac, PSMA I&T labeled for example with ¹⁷⁷Lu or ²²⁵Ac, FrhPSMA-7 labeled for example with ¹⁷⁷Lu, 64/67Cu-SAR-bisPSMA (Clarity Pharmaceuticals), CONV 01-α (Convergent Therapeutics, Inc.) labeled for example with ²²⁵Ac, ¹⁷⁷Lu-PSMA I&T-β+²²⁵Ac-CONV01-α combination (Convergent Therapeutics, Inc.), ¹³¹I-1095 (Lantheus Holdings/Progenics Pharmaceuticals, Inc.), ¹³¹I PSMA-PK-Rx (Noria Therapeutics, Inc.; Bayer), or PSMA-R2 labeled for example with ¹⁷⁷Lu, CTT1403 (Cancer Targeted Technology LLC) labeled for example with ¹⁷⁷Lu, PNT2002/Lu-177-PSMA-I&T (Point Biopharma Global Inc.), PNT2002/Lu-177-PSMA-I&T+²²⁵Ac-J591, TLX591 (¹⁷⁷Lu-Rosopatamab; Telix Pharmaceuticals Ltd.), TLX-591-CHO (Telix Pharmaceuticals Ltd.), and ¹⁷⁷Lu-EB-PSMA-617 (Sinotau Radiopharmaceutical). Such agents may, for example, be used in the treatment of prostate cancer, such as metastatic prostate cancer, castration-resistant prostate cancer (CRPC), metastatic CRPC (mCRPC), and/or hormone therapy resistant prostate cancer (anti-androgen therapy resistant prostate cancer) in combination with or in conjunction with a radiolabeled NKG2DL targeting agent according to the invention. Any of the agents that include DOTA or a DOTA derivative as a chelator may alternatively be labeled with any therapeutically active radionuclide that can be chelated by DOTA, such as ²²⁵Ac, ¹⁷⁷Lu and ⁹⁰Y.

A radiolabeled cancer targeting agent used in combination or conjunction with a radiolabeled NKG2DL targeting agent, such as any of those disclosed herein, may, for example, be any of the following radiolabeled targeting agents, or any combination thereof:
a radiolabeled FAP targeting agent such as ¹⁷⁷Lu-FAP-2286 (Clovis Oncology, Inc.) to treat, for example, solid tumors or any of the cancers disclosed herein;
a radiolabeled CCK2R targeting agents such as DEBIO 1124/¹⁷⁷Lu-DOTA-PP-F11N (Debiopharm International SA) to treat, for example, advanced, unresectable pulmonary extrapulmonary small cell carcinoma, and thyroid cancer such as metastatic thyroid cancer, or any of the cancers disclosed herein;
a radiolabeled CDH3 (cadherin-3, P-cadherin) targeting agent such as ⁹⁰Y labeled FF-21101 (FujiFilm Holdings Corporation/FujiFilm Toyama Chemical) to treat, for example, solid tumors such as epithelial ovarian peritoneal or fallopian tube carcinoma, TNBC, head and neck squamous cell carcinoma (HNSCC), cholangiocarcinoma, pancreatic, colorectal cancer, or any of the cancers disclosed herein;
a radiolabeled IGF-R1 targeting agent such as ²²⁵Ac FPI-1434 (Fusion Pharmaceuticals, Inc.) to treat, for example, solid tumors expressing IGF-R1, or any of the cancers disclosed herein;
a radiolabeled CEACAM5 targeting agent such as ⁹⁰Y-hMN14 and ⁹⁰Y TF2 (Immunomedics, Inc.; Gilead Sciences Inc.) to treat, for example, solid tumors such as colon cancer, colorectal cancer, pancreatic cancer, breast cancer such as HER-negative breast cancer, and thyroid cancer such medullary thyroid carcinoma, or any of the cancers disclosed herein;
a radiolabeled CD22 targeting agent such as IMMU-102 (⁹⁰Y-epratuzumab) (Immunomedics, Inc.; Gilead Sciences Inc.) to treat, for example, hematological malignancies such as CD22-positive acute lymphoblastic leukemia, non-Hodgkin lymphoma (NHL), stage III/IV DLBCL, follicular lymphoma, or any of the cancers disclosed herein;
a radiolabeled SSTR2 targeting agent such as Lutathera™ (lutetium Lu 177 dotatate; 177Lu-DOTAO-Tyr3-Octreotate; Novartis), Lutathera™ (lutetium Lu 177 dotatate) plus ⁹⁰Y-DOTATATE combination (Novartis), ¹⁷⁷LU-OPS201 (Ipsen Pharmaceuticals) the combination ¹⁷⁷LU-OPS201/¹⁷⁷Lu-IPN01072 (Ipsen Pharmaceuticals), EBTATE (¹⁷⁷Lu-DOTA-EB-TATE; Molecular Targeting Technologies, Inc.), ORM2110 (AlphaMedix™; Orano Med), and PNT2003 labeled for example with ¹⁷⁷Lu (Point Biopharma Global Inc.), for the treatment of SSTR2 expressing cancers such as solid tumors, for example, neuroendocrine tumors, small cell lung cancer, breast cancer, prostate cancer such as metastatic prostate cancer, such as metastatic castration-resistant prostate cancer, neuroendocrine tumors, gastroenteropancreatico neuroendocrine tumors (GEP-NET), as well as such as locally advanced or metastatic forms thereof, or any of the cancers disclosed herein;
a radiolabeled SSTR2 and SSTR5 targeting agent such as Solucin™ (¹⁷⁷Lu-Edotreotide; Isotopen Technologien Munchen AG (ITM)) to treat, for example, neuroendocrine tumors, or any of the cancers disclosed herein;
a radiolabeled Neurotensin receptor type 1 (NTSR1) targeting agent such as ¹⁷⁷Lu-IPN01087/¹⁷⁷Lu-3BP-227 or (Ipsen Pharmaceuticals) to treat, for example, solid tumors expressing NTSR1 such as pancreatic ductal adenocarcinoma, colorectal cancer, gastric cancer, squamous cell carcinoma of the head and neck, bone cancer, advanced cancer, recurrent disease, metastatic tumors, or any of the cancers disclosed herein;
a radiolabeled human Kallikrein-2 (hK2) targeting agent such as JNJ-69086420 (Janssen/Janssen Pharmaceutica NV) labeled for example with ²²⁵Ac, to treat, for example, prostate cancer such as locally advance or metastatic prostate cancer, or any of the cancers disclosed herein;
a radiolabeled NET (via norepinephrine transporter) targeting agent such as ¹³¹I-MIBG (Jubilant Radioharma) to treat, for example, neuroblastoma such as relapsed/refractory neuroblastoma, or any of the cancers disclosed herein;
a radiolabeled neuroepinephrine transporter targeting agents such as Azedra™ (iobenguane ¹³¹I; Lantheus Holdings/Progenics Pharmaceuticals, Inc.) to treat, for example, glioma, paraglioma, malignant pheochromocytoma/paraganglioma, and malignant relapsed/refractory pheochromocytoma/paraganglioma, or any of the cancers disclosed herein;
a radiolabeled Integrin αVβ6 targeting agent such as DOTA-ABM-5G, αVβ6 Binding Peptide (ABP; Luminance Biosciences, Inc.) labeled for example with ¹⁷⁷Lu, ²²⁵Ac or ⁹⁰Y, to treat, for example, solid tumors such as pancreatic cancer, or any of the cancers disclosed herein;
a radiolabeled CD37 targeting agent such as Betalutin™ (¹⁷⁷Lu-lilotomab satetraxetan; Nordic Nanovector ASA) to treat, for example, hematological malignancies such as lymphomas, such as follicular lymphoma or non-Hodgkin lymphoma (NHL) such as relapsed and/or refractory forms thereof, or any of the cancers disclosed herein;
a radiolabeled GRPR targeting agent such as ¹⁷⁷Lu-NeoB (Novartis) and ²¹²Pb-DOTAM-GRPR1 (Orano Med) to treat GRPR-expressing cancers, for example, prostate cancer, such as advanced prostrate cancer, locally advanced prostate cancer, metastatic prostate cancer, and castration-resistant prostate cancer, or any of the cancers disclosed herein;
a radiolabeled CXCR4 targeting agents such as PentixaTher™ (PentixaPharm GmbH) labeled with ¹⁷⁷Lu, ⁹⁰Y or ²²⁵Ac to treat, for example, lymphoproliferative or myeloid malignancies, including relapsed and/or refractory forms thereof, or any of the cancers disclosed herein;
a radiolabeled Tenascin-C targeting agent such as ¹³¹I-F16SIP (Philogen S.p.A.) to treat, for example, solid tumors or hematological malignancies such as any of those disclosed herein;
a radiolabeled Fibronectin extradomain B (EBD) targeting agent such as ¹³¹I-L19SIP (Philogen S.p.A.) to treat, for example, solid tumors such as solid tumor brain metastases and non-small cell lung cancer (NSCLC), or any of the cancers disclosed herein;
a radiolabeled LAT-1 targeting agent such as 4-¹³¹Iodo-L-phenylalanine (Telix Pharmaceuticals Ltd.) to treat, for example, glioblastoma such as recurrent glioblastoma, or any of the cancers disclosed herein;
a radiolabeled Carbonic Anhydrase IX (CAIX) targeting agent such as radiolabeled Girentuxumab (cG250) such as DOTA conjugated Girentuxumab (cG250) labeled for example with ¹⁷⁷Lu (such as TLX250; Telix Pharmaceuticals Ltd.), ²²⁵Ac or ⁹⁰Y, to treat, for example, renal cell carcinoma, such as clear cell renal cell carcinoma (ccRCC), or any of the cancers disclosed herein;
a radiolabeled CD66 targeting agent such as ⁹⁰Y-besilesomab (⁹⁰Y-anti-CD66-MTR; Telix Pharmaceuticals Ltd.) to treat, for example, leukemias, myelomas and lymphomas, such as any of those disclosed herein including pediatric and adult forms, or any of the cancers disclosed herein;
a radiolabeled B7-H3 targeting agents such as radiolabeled omburtumab, such ¹³¹I-8H9 (131I-omburtumab; Y-mAbs Therapeutics, Inc.) and ¹⁷⁷Lu-omburtamab (Y-mAbs Therapeutics, Inc.) to treat, for example, gliomas such as non-progressive diffuse pontine gliomas, such as non-progressive diffuse pontine gliomas previously treated with external beam radiation therapy, brain tumors, central nervous system tumors, neuroblastomas, sarcomas, leptomeningeal metastasis from solid tumors, and medulloblastoma, including in pediatric and adult forms, or any of the cancers disclosed herein;
a radiolabeled GD2 targeting agent such as GD2-SADA:¹⁷⁷Lu-DOTA (Y-mAbs Therapeutics, Inc.) to treat, for example, SCLC, melanoma, sarcoma or any of the cancers disclosed herein;
a radiolabeled Folate receptor alpha (FOLR1) targeting agent such as a radiolabeled anti-FOLR1 antibody such as radiolabeled Mirvetuximab or Farletuzumab, to treat, for example, solid cancers such as ovarian cancer, lung cancer, NSCLC, breast cancer, TNBC, brain cancer, glioblastoma, colorectal cancer or any of the cancers disclosed herein;
a radiolabeled Nectin-4 targeting agent, such as a radiolabeled anti-Nectin-4 monoclonal antibody such as radiolabeled Enfortumab or radiolabeled forms of any of the anti-Nectin-4 antibodies or targeting agents disclosed in U.S. Pub. No. 20210130459, U.S. Pub. No. 20200231670, U.S. Pat. No. 10,675,357, or Int'l Pub. No. WO2022051591, to treat, for example, solid tumors such as urothelial carcinoma, bladder carcinoma, breast cancer, TNBC, lung cancer, NSCLC, colorectal cancer, pancreatic cancer, endometrial cancer, ovarian cancer or any of the cancers disclosed herein;

a radiolabeled CUB-domain containing protein 1 (CDCP1) targeting agent such as a radiolabeled monoclonal antibody such as radiolabeled forms of any of the CDCP1 targeting agents and antibodies disclosed in U.S. Pub. No. 20210179729, U.S. Pub. No. 20200181281, U.S. Pub. No. 20090196873, U.S. Pat. Nos. 8,883,159, 9,346,886, or Int'l Pub No. WO2021087575, to treat, for example, solid cancers such as breast cancer, TNBC, lung cancer, colorectal cancer, ovarian cancer, kidney cancer, liver cancer, HCC, pancreatic cancer, skin cancer, melanoma, or a hematological malignancy such as acute myeloid leukemia, or any of the cancers disclosed herein;

a radiolabeled Glypican-3 (GPC3) targeting agent such as a radiolabeled anti-GPC3 mAb such as the radiolabeled humanized IgG₁ mAb GC33 (a/k/a Codrituzumab; commercially available as Catalog No. TAB-H14 from Creative Biolabs), such as ²²⁵Ac-Macropa-GC33 (Bell et al., Glypican-3-Targeted Alpha Particle Therapy for Hepatocellular Carcinoma. Molecules. 2020 Dec. 22; 26(1):4.) or a radiolabeled form of any of the anti-GPC3 antibodies or other targeting agents disclosed in U.S. Pat. Nos. 10,118,959, 10,093,746, 10,752,697, 9,932,406, 9,217,033, 8,263,077, 7,871,613, 7,867,734, U.S. Pub. No. 20190046659, U.S. Pub. No. 20180243451, U.S. Pub. No. 20170369561, or U.S. Pub. No. 20150315278, to treat GPC3-expressing cancers such as hepatocellular carcinoma, ovarian clear cell carcinoma, melanoma, NSCLC, squamous cell carcinoma of the lung, hepatoblastoma, nephroblastoma (Wilms tumor), yolk sac tumor, gastric carcinoma, colorectal carcinoma, head and neck cancer, and breast cancer.

a radiolabeled urokinase plasminogen activator receptor (uPAR) targeting agent, such as a radiolabeled monoclonal antibody such as radiolabeled MINPR-101 (huATN-658) such as MNPR-101-PTCA-Ac225 (Monopar Therapeutics, Inc., Wilmette, IL, USA) or radiolabeled forms of any of the anti-uPAR antibodies or targeting agents disclosed in U.S. Pat. No. 9,029,509, U.S. Pub. No. 20080199476, U.S. Pub. No. 20040204348 or Int'l Pub. No. WO2021257552, to treat, for example, solid cancers or hematological malignancies such as any of those disclosed herein;

a radiolabeled FGFR2 targeting agent, such as a radiolabeled FGFR2b targeting agent, such as a radiolabeled anti-FGFR2b antibody, such as radiolabeled Bemarituzumab (Amgen; Five Prime Therapeutics) or radiolabeled forms of any of the FGFR2b antibodies or other targeting agents disclosed in any of U.S. Pat. No. 9,481,733 (Daiichi Sankyo Co Ltd), U.S. Pub. No. 20140322220 (Bayer), U.S. Pat. No. 9,931,401 (Daiichi Sankyo Co Ltd), U.S. Pub. No. 20130288305 (Aveo Pharmaceuticals, Inc.), and U.S. Pub. No. 20220041737A1 (Five Prime Therapeutics, Inc), to treat, for example, gastric cancer and gastroesophageal junction cancer, esophageal cancer, colorectal cancer, pancreatic cancer, hepatocellular carcinoma, and breast cancer, or any of the cancers disclosed herein;

a radiolabeled Six-transmembrane epithelial antigen of prostate 1 (STEAP1) targeting agent, such as a radiolabeled anti-STEAP1 antibody, such as radiolabeled forms of any of the STEAP1 antibodies or other targeting agents disclosed in any of U.S. Pub. No. 20210179731 (Amgen; Xencor), U.S. Pat. No. 10,017,577 (Genentech), Int'l Pub. No. WO2021046331A1 (Memorial Sloan Kettering Cancer Center), and U.S. Pub. No. 20210277148 (Amgen), to treat, for example, STEAP1 expressing cancers such as prostate cancer, metastatic castration-resistant prostate cancer, leukemia, lymphoma, colorectal cancer, esophageal carcinoma, lung carcinoma, diffuse large B cell lymphoma, acute myeloid leukemia, multiple myeloma, acute lymphoblastic T cell leukemia, diffuse large B cell lymphoma, Hodgkin lymphoma, or any of the cancers disclosed herein;

a radiolabeled BCMA targeting agent, such as a radiolabeled anti-BCMA antibody, such as radiolabeled forms of any of the BCMA antibodies or other targeting agents disclosed in any of U.S. Pat. No. 9,243,058 (Amgen), U.S. Pat. No. 9,340,621 (Amgen), and Int'l Pub. No. WO2017031104 (Janssen), to treat, for example, multiple myeloma, chronic lymphocytic leukemia, acute B-lymphoblastic leukemia, non-Hodgkin lymphoma (NHL), Hodgkin lymphoma, or any of the cancers disclosed herein;

a radiolabeled MUC17 targeting agent, such as a radiolabeled anti-MUC17 antibody, such as radiolabeled forms of any of the MUC17 antibodies or other targeting agents disclosed in U.S. Pub. No. 20210130465 (Amgen) or U.S. Pat. No. 8,546,546 (Chugai Pharmaceuticals), to treat, for example, MUC17 expressing cancers such as gastric cancer, gastroesophageal junction cancer, colorectal cancer, and pancreatic cancer;

a radiolabeled CDLN18.2 targeting agent, such as a radiolabeled anti-CDLN18.2 antibody, such as radiolabeled forms of any of the CDLN18.2 antibodies or other targeting agents disclosed in any of U.S. Pat. No. 10,314,890 (Astellas), U.S. Pub. No. 20180117174, U.S. Pub. No. 20200207857 (Spark Therapeutics), U.S. Pub. No. 20180258180 (Ganymed Pharmaceuticals, GmbH), U.S. Pub. No. 20210230272, U.S. Pub. No. 20210009686, and Chinese Pub. No. CN112770723A (Akeros Bioscience Co), to treat, for example, CLDN18.2-expressing cancers such as gastric cancer, gastrointestinal tract cancers, gastroesophageal junction cancer, esophageal cancer, pancreatic cancer, lung cancer, lung adenocarcinoma, and any of the cancers disclosed herein;

a radiolabeled Sortilin (Neurotensin receptor-3) targeting agent, such as a radiolabeled anti-Sortilin antibody, such as radiolabeled forms of any of the Sortilin 2 antibodies or other targeting agents disclosed in any of U.S. Pat. No. 11,186,645 (Alector), U.S. Pat. No. 10,428,147 (Lundbeck), U.S. Pub. No. 20200024348A1 (Adimab; Alector), and U.S. Pub. No. 20200392229A1 (Alector), to treat, for example, Sortilin-expressing cancers such as breast cancer, lung cancer, thyroid cancer, glioblastoma, colorectal cancer, or pancreatic cancer such as any of the forms disclosed herein, or any of the cancers disclosed herein; and/or

a radiolabeled LewisY antigen (LeY) targeting agent such as a radiolabeled anti-LeY monoclonal antibody such as radiolabeled forms of 3S1931 and/or of a humanized version thereof such as Hu3S1933, or of any of monoclonal antibodies B34, BR55-2, BR55/BR96, and IGN 133, or antigen binding fragments of any of the preceding antibodies, to treat, for example, solid tumors such as squamous cell lung carcinoma, lung adenocarcinoma, ovarian carcinoma, or colorectal adenocarcinoma or any of the cancers disclosed herein.

In still further embodiments of the invention, a radiolabeled targeting agent used in combination or conjunction with a radiolabeled NKG2DL targeting agent for the treatment of a cancer or proliferative disorder such as any of those disclosed herein in a mammal, such as a human, includes a phospholipid-based cancer targeting agent. In certain embodiments, the phospholipid-based cancer targeting agent includes any of the radioactive phospholipid metal chelates disclosed in U.S. Pub. No. 20200291049, incorporated by reference herein, such as but not limited to

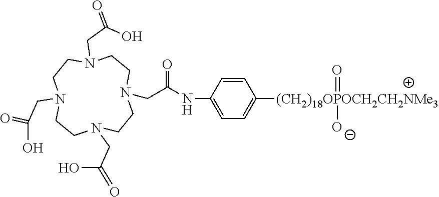

(a/k/a NM600) or a pharmaceutically acceptable salt thereof, chelated with a radionuclide, such as ²²⁵Ac, ¹⁷⁷Lu, or ⁹⁰Y.

In certain aspects, the lipid based radiolabeled targeting agent used in conjunction with a radiolabeled NKG2DL targeting agent includes any of the radiolabeled phospholipid compounds disclosed in U.S. Pub. No. 20140030187 or U.S. Pat. No. 6,417,384, each incorporated by reference herein, such as but not limited to

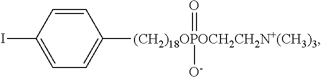

i.e., 18-(p-iodophenyl)octadecyl phosphocholine, wherein iodine is ¹³¹I (a/k/a NM404 I-131, and CLR 131), or a pharmaceutically acceptable salt thereof. In certain aspects, the phospholipid-based radiolabeled targeting agent used in conjunction with one or more CD47 blockades includes any of the phospholipid drug conjugate compounds disclosed in U.S. Pat. No. 9,480,754, incorporated by reference herein.

### EXAMPLES

### Example 1: Production of Radioconjugated NKG2DL Targeting Agent

The NKG2DL targeting agent, such as a monoclonal antibody against an NKG2DL such as MICA and/or MICB or an antigen-binding fragment thereof or a soluble NKG2D homodimer, such as an NKG2D-Fc fusion protein, may be labeled with a metallic radionuclide (radiometal) such as ⁶⁷Ga, ⁶⁸Ga, ^{99m}Tc, ¹¹¹In, ^{114m}In, ¹⁷⁷Lu, ⁶⁴Cu, ⁴⁴Sc, ⁴⁷Sc, ⁸⁶Y, ⁹⁰Y, ⁸⁹Zr, ²¹²Bi, ²¹³Bi, ²¹²Pb, ²²⁵Ac, ²²⁷Th, ¹⁸⁶Re or ¹⁸⁸Re. Radionuclides that may be used for diagnostic purposes include but are not limited to ⁶⁷Ga, ^{99m}Tc, ¹¹¹In, and ¹⁷⁷Lu, which are useful in single photon emission computed tomography (SPECT), and ⁶⁸Ga, ⁶⁴Cu, ⁴⁴Sc, ⁸⁶Y, and ⁸⁹Zr, which are useful in positron emission tomography (PET). Radionuclides that may be used for therapeutic purposes include but are not limited to ⁴⁷Sc, ^{114m}In, ¹⁷⁷Lu, ⁹⁰Y, ²¹²Bi, ²¹³Bi, ²¹²Pb, ²²⁵Ac, ²²⁷Th, ¹⁶Re, and ¹⁸⁸Re.

The NKG2DL targeting agent and optionally other targeting agent(s) may, for example, be labeled with Iodine-131 (¹³¹I) or other Iodine isotopes according to the radio-iodination procedures detailed in International Pub. No. WO 2017155937 and U.S. Pat. No. 10,420,851 or with Actinium-225 (²²⁵Ac) or Lutetium-177 (¹⁷⁷Lu), each of which can be chelated by DOTA, according to procedures described in U.S. Pat. No. 9,603,954. Specific chelator conjugation and radiolabeling methods for proteins such as antibodies and soluble receptors and peptides (e.g., that include primary amines such as from lysine residues) are exemplified below with respect to an anti-NKG2DL antibody.

Preparing the DOTA-conjugated antibody using p-SCN-Bn-DOTA: antibody conjugates may be prepared by reacting a concentrated solution of monoclonal anti-NKG2DL antibody with p-SCN-Bn-DOTA in bicarbonate or in phosphate buffers at pH between about 8 and about 9 and by incubation at either about 37° C. or at room temperature. The conjugates may be purified from excess of the bifunctional chelator by repeated filtration or centrifugation and by gravity size exclusion chromatography (SEC). During the purification process, the bicarbonate or phosphate buffer is changed to N-2-Hydroxyethylpiperazine-N-2-ethanesulfonic acid (HEPES; Free Acid) or acetate medium. Conjugates may be characterized by size exclusion high performance liquid chromatography (SE-HPLC).

Preparing the DOTA-conjugated antibody using PODS-DOTA: DOTA may be conjugated to a monoclonal antibody, such as an IgG, or an antigen-binding portion thereof, using PODS-DOTA in the presence of TCEP, a mild reducing agent that cleaves the inter-chain disulfide bonds within an immunoglobin according to the methods set forth in U.S. Pat. No. 11,000,604. The structure of PODS-DOTA is

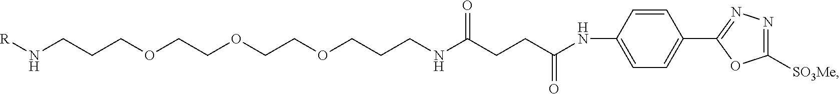

wherein R is a covalently bound DOTA moiety.

In more detail, to a suspension of 200 μg of antibody in PBS pH 7.4 (1 mg/mL) 1.33 μL of a fresh TCEP solution (10 mM in water, 10 eq.) is added and the appropriate volume of a solution of PODS-DOTA (1 mM in DMSO). The reaction mixture is then stirred on a thermomixer (25° C. or 37° C.) for 30 min, 2 h, or 24 h. The conjugate is then purified on a size exclusion column (Sephadex G-25 M, PD-10 column, GE Healthcare; dead volume=2.5 mL, eluted with 2 mL of PBS, pH 7.4) and concentrated using centrifugal filtration units with a 50,000 Da molecular weight cut off (AMICON™ Ultra 4 Centrifugal Filtration Units, Millipore Corp. Billerica, Mass.)

Radiolabeling the DOTA-conjugated antibody: The NKG2DL targeting agent, such as antibody, may be conjugated to a chelator-bearing linker, such as any of the linkers described in the above indicated patent documents or as exemplified above. An exemplary linker includes at least dodecane tetraacetic acid (DOTA) or a derivative thereof, wherein a goal of the conjugation reaction is to achieve a DOTA-antibody ratio of 3:1 to 5:1. Chelation with the radionuclide (e.g., ¹⁷⁷Lu or ²²⁵Ac) may then be performed and efficiency and purity of the resulting radioconjugated anti-NKG2DL antibody may be determined by HPLC and iTLC.

An exemplary labeling reaction for ²²⁵Ac is as follows: A reaction including 15 μl 0.15M NH₄OAc buffer, pH=6.5 and 2 μL (10 g) DOTA-anti-NKG2DL (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 minutes with shaking at 100 rpm. At the end of the incubation period, 3 μL of a 1 mM DTPA solution may be added to the reaction mixture and incubated at room temperature for 20 minutes to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-NKG2DL through separating ²²⁵Ac-labeled anti-NKG2DL (²²⁵Ac-DOTA-anti-NKG2DL) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: An exemplary radioconjugated NKG2DL targeting agent, such as ²²⁵Ac-DOTA-anti-NKG2DL, may be purified either on PD10 columns pre-blocked with 1% HSA or on Vivaspin centrifugal concentrators with a 50 kDa MW cut-off with 2×1.5 mL washes, 3 minutes per spin. HPLC analyses of the ²²⁵Ac-DOTA-anti-NKG2DL after purification may be conducted using a Waters HPLC system equipped with flow-through Waters UV and Bioscan Radiation detectors, using a TSK3000SW XL column eluted with PBS at pH=7.4 and a flow rate of 1 ml/min.

Stability determination: An exemplary radioconjugated NKG2DL targeting agent, such as ²²⁵Ac-DOTA-anti-NKG2DL, may be used for stability determination, wherein the ²²⁵Ac-DOTA-anti-NKG2DL may be tested either in the original volume or diluted (2-10 fold) with the working buffer (0.15 M NH₄OAc) and incubated at room temperature (rt) for 48 hours or at 4° C. for 96 hours and tested by ITLC. Stability is determined by comparison of the intact radiolabeled anti-NKG2DL before and after incubation. Other antibodies labeled with ²²⁵Ac have been found to be stable at 4° C. for up to 96 hrs.

Immunoreactivity (IR) determination: An exemplary radioconjugated NKG2DL targeting agent, such as ²²⁵Ac-DOTA-anti-NKG2DL, may be used in immunoreactivity experiments. NKG2DL positive cells and control NKG2DL negative cells may be used in the amounts of 1.0-7.5 million cells per sample to investigate the amount of binding (percent radioactivity binding to cells after several washes; or using an immunoreactive fraction (IRF) bead assay may be performed according to methods disclosed in as described by Sharma, 2019). Prior assays for other antibodies radiolabeled with ¹¹¹In or ²²⁵Ac demonstrated about 50-60% immunoreactivity.

### Example 2: Methods for Testing the Radioconjugated NKG2DL Targeting Agent as a Single Modality—a Xenograft Model of Human Colorectal Cancer Using the HCT116 Cell Line (a Solid Tumor Model)

Biodistribution of Radioconjugated Anti-MICA mAb: Immunodeficient NSG mice will be injected subcutaneously in the right flank with 2.5×10⁶ HCT116 cells. When tumors grow to approximately 100 mm³, mice will be treated intravenously with anti-MICA mAb conjugated with ¹¹¹In, a gamma-emitting radioisotope that does not cause DNA damage or cancer cell death. Blood, tumor, and major organs will be harvested 4, 24, 48, 96, and 168 hours post ¹¹¹In-anti-MICA administration for dosimetry calculations in which the absorbed dose of radiation is quantified for each tissue and time point. ¹¹¹In-anti-MICA should home to tumor tissue but not in healthy tissue because only the transplanted tumors would express MICA.

Dose Escalation Study with ²²⁵Ac-anti-MICA: ²²⁵Ac causes DNA damage and cancer cell death. Immunodeficient NSG mice will be injected subcutaneously in the right flank with 2.5×10⁶ HCT116 cells. When tumors grow to approximately 100 mm³, mice will be treated intravenously with various doses of ²²⁵Ac-conjugated anti-MICA antibody (0, 100, 200, 400, 500 nCi ²²⁵Ac; 500 ng total amount of IgG). This dose escalation study will determine the maximum tolerated dose (MTD) and minimum effective dose (MED). MTD will be defined as the highest dose that permits all treated mice to maintain weight above 85% of the baseline weight, and MED will be defined as the minimum dose that leads to quantifiable shrinkage of the tumors. Total body weights and tumor volumes will be measured twice a week, and a Kaplan-Meier survival curve will be generated.

### Example 3: Antibody Dose—Radioconjugated Versus Unconjugated

Conjugating an antibody with ²²⁵Ac would substantially decrease the amount of total antibody necessary to achieve tumor response. Based on previous experience comparing the efficacy of ²²⁵Ac conjugated- and unconjugated monoclonal antibodies (Dawicki, 2019), the amount of anti-NKG2DL antibody required to elicit tumor response may be decreased approximately 30-fold if conjugated with ²²⁵Ac. Furthermore, due to the potency of the alpha-emitter, a single administration of the radioconjugated agent should be sufficient to observe tumor reduction. However, because biological responses to antitumor therapy are difficult to predict, we will also test hypofractionated regimens, where the total radiation dose is divided into two or three administrations to determine which schedule is optimal.

### Example 4: Method for Testing the Feed-Forward Mechanism of Tumor Eradication

Anti-MICA antibody conjugated with a DNA damage-inducing radioisotope could lead to a feed-forward mechanism that further enhances the accumulation of the therapeutic agent within the tumor. The hypothetical molecular underpinnings are as follows: Radiation from the radio-isotope up-regulates MICA expression, which then leads to more targeted binding of the therapeutic agent specifically within the tumor. The ionizing radiation is a crucial component of the feed-forward mechanism. To experimentally corroborate this model, we will radioconjugate anti-MICA antibody with ²²⁵Ac or ¹¹¹In and measure the tumor accumulation of radiation over time. Since ²²⁵Ac induces DNA damage via ionizing radiation whereas ¹¹¹In does not cause DNA damage, only ²²⁵Ac-anti-MICA can up-regulate MICA within the tumor and cause target accumulation.

Immunodeficient NSG mice will be injected subcutaneously in the right flank with 2.5×10⁶ HCT116 cells. When tumors grow to approximately 200 mm³, mice will be treated intravenously with ²²⁵Ac- or ¹¹¹In-conjugated anti-MICA antibody (MTD nCi; 500 ng total amount of IgG). Mice will then be euthanized at 24, 48, 96, 192, and 384 hours post injection, and tumors will be dissected and weighed for scintillation counting. If ²²⁵Ac-anti-MICA induces MICA expression within the tumor, this would be manifested through a higher overall accumulation of radiation per gram tissue over time compared to ¹¹¹In-anti-MICA because a greater number of target sites are available for antibody binding. In contrast, ¹¹¹In-anti-MICA does not induce ionizing radiation and would not elevate the expression of MICA, leading to a more muted accumulation compared to ²²⁵Ac-anti-MICA.

In a separate experiment, mice will be treated as described above, and tumor volumes will be measured at 24, 48, 96, 192, and 384 hours post ²²⁵Ac-anti-MICA or ¹¹¹In-anti-MICA administration. Then, the samples will be subjected to immunohistochemistry to detect expression of MICA. Since ²²⁵Ac-anti-MICA would initiate a feed-forward mechanism of MICA induction but ¹¹¹In-anti-MICA would not, only the ²²⁵Ac-anti-MICA treated mice would exhibit intratumoral MICA expression that increases over time, paralleling a substantial decrease in tumor volume from baseline. As an alternative to immunohistochemistry, western blot can be performed to detected MICA.

### Example 5: Methods for Testing Therapeutic Combination Approaches

(A) Combining Radioconjugated Anti-MICA mAb with DDR Inhibitor

In vivo: Immunodeficient NSG mice will be injected subcutaneously in the right flank with 2.5×10⁶ HCT116 cells. When tumors grow to approximately 100 mm³, mice will be treated intravenously with various doses of radioconjugated anti-MICA antibody (0, MED, and MTD ²²⁵Ac; 500 ng total amount of IgG), in the presence or absence of a DDR inhibitor, which may include but not be limited to a PARP inhibitor, ATM or ATR inhibitor, or Wee1 inhibitor. Total body weights and tumor volumes will be measured twice a week, and a Kaplan-Meier survival curve will be generated to determine if this combinatorial approach extends overall survival.

(B) Combining Radioconjugated Anti-MICA mAb with CD47 Blockade

In vivo: Immunodeficient NSG mice will be injected subcutaneously in the right flank with 2.5×10⁶ HCT116 cells. When tumors grow to approximately 100 mm³, mice will be treated intravenously with various doses of radioconjugated anti-MICA antibody (0, MED, and MTD ²²⁵Ac; 500 ng total amount of IgG), in the presence or absence of an antibody that blocks the function of CD47. Total body weights and tumor volumes will be measured twice a week, and a Kaplan-Meier survival curve will be generated to determine if this combinatorial approach extends overall survival. In a separate experiment, tumor infiltration of immune cells (e.g., monocytes, macrophages, T cells, NK cells, neutrophils) can be quantified using flow cytometry to determine if addition of CD47 blockade activates an antitumor immune response.

(C) Combining Radioconjugated Anti-MICA mAb with ICI

In vivo: Immunodeficient NSG mice will undergo sublethal total body irradiation and be inoculated intravenously with 2×10⁷ human peripheral blood lymphocytes. After confirmation of successful engraftment, mice will be injected subcutaneously in the right flank with 2.5×10⁶ HCT116 cells. When tumors grow to approximately 100 mm³, mice will be treated intravenously with various doses of radioconjugated anti-MICA antibody (0, MED, and MTD ²²⁵Ac; 500 ng total amount of IgG), in the presence or absence of an antibody that blocks the function of human PD-L1 or PD-1, i.e., an immune checkpoint inhibitor (ICI). Weights and tumor volumes will be measured twice a week, and a Kaplan-Meier survival curve will be generated. In a separate experiment, tumor infiltration of immune cells (e.g., monocytes, macrophages, T cells, NK cells, neutrophils) can be quantified using flow cytometry to determine if addition of PD-1/PD-L1 blockade activates an antitumor immune response.

### Example 6—Exemplary PARPi Administration and Dosing Regimes

### (A) Olaparib (Lynparza®)—Normal and Reduced Dosing Regimens

Olaparib is sold by AstraZeneca under the brand name Lynparza®. Lynparza® is sold in tablet form at 100 mg and 150 mg. The dosage is 300 mg taken orally twice daily for a daily total of 600 mg. Dosing continues until disease progression or unacceptable toxicity. This dosing regimen is referred to herein as the “normal” human dosing regimen for Lynparza®, regardless of the disorder treated. Any dosing regimen having a shorter duration (e.g., 21 days) or involving the administration of less Lynparza® (e.g., 300 mg/day) is referred to herein as a “reduced” human dosing regimen. Examples of reduced human dosing regimens include the following: (i) 550 mg/day; (ii) 500 mg/day; (iii) 450 mg/day; (iv) 400 mg/day; (v) 350 mg/day; (vi) 300 mg/day; (vii) 250 mg/day; (viii) 200 mg/day; (ix) 150 mg/day; (x) 100 mg/day; or (xi) 50 mg/day.

### (B) Niraparib (Zejula®)—Normal and Reduced Dosing Regimens

Niraparib is sold by Tesaro under the brand name Zejula®. Zejula® is sold in capsule form at 100 mg. The dosage is 300 mg taken orally once daily. Dosing continues until disease progression or unacceptable adverse reaction. This dosing regimen is referred to herein as the “normal” human dosing regimen for Zejula®, regardless of the disorder treated. Any dosing regimen having a shorter duration (e.g., 21 days) or involving the administration of less Zejula® (e.g., 150 mg/day) is referred to herein as a “reduced” human dosing regimen. Examples of reduced human dosing regimens include the following: (i) 250 mg/day; (ii) 200 mg/day; (iii) 150 mg/day; (iv) 100 mg/day; or (v) 50 mg/day.

### (C) Rucaparib (Rubraca®)—Normal and Reduced Dosing Regimens

Rucaparib is sold by Clovis Oncology, Inc. under the brand name Rubraca™ Rubraca™ is sold in tablet form at 200 mg and 300 mg. The dosage is 600 mg taken orally twice daily for a daily total of 1,200 mg. Dosing continues until disease progression or unacceptable toxicity. This dosing regimen is referred to herein as the “normal” human dosing regimen for Rubraca™, regardless of the disorder treated. Any dosing regimen having a shorter duration (e.g., 21 days) or involving the administration of less Rubraca™ (e.g., 600 mg/day) is referred to herein as a “reduced” human dosing regimen. Examples of reduced human dosing regimens include the following: (i) 1,150 mg/day; (ii) 1,100 mg/day; (iii) 1,050 mg/day; (iv) 1,000 mg/day; (v) 950 mg/day; (vi) 900 mg/day; (vii) 850 mg/day; (viii) 800 mg/day; (ix) 750 mg/day; (x) 700 mg/day; (xi) 650 mg/day; (xii) 600 mg/day; (xiii) 550 mg/day; (xiv) 500 mg/day; (xv) 450 mg/day; (xvi) 400 mg/day; (xvii) 350 mg/day; (xviii) 300 mg/day; (xix) 250 mg/day; (xx) 200 mg/day; (xxi) 150 mg/day; or (xxii) 100 mg/day.

### (D) Talazoparib (Talzenna™)—Normal and Reduced Dosing Regimens

Talazoparib is sold by Pfizer Labs under the brand name Talzenna™. Talzenna™ is sold in capsule form at 1 mg. The dosage is 1 mg taken orally. Dosing continues until disease progression or unacceptable toxicity. This dosing regimen is referred to herein as the “normal” human dosing regimen for Talzenna™, regardless of the disorder treated. Any dosing regimen having a shorter duration (e.g., 21 days) or involving the administration of less Talzenna™ (e.g., 0.5 mg/day) is referred to herein as a “reduced” human dosing regimen. Examples of reduced human dosing regimens include the following: (i) 0.9 mg/day; (ii) 0.8 mg/day; (iii) 0.7 mg/day; (iv) 0.6 mg/day; (v) 0.5 mg/day; (vi) 0.4 mg/day; (vii) 0.3 mg/day; (viii) 0.2 mg/day; or (ix) 0.1 mg/day.

### Example 7: Dosing Regimens for a NKG2DL Targeting Agent and PARPi

A human patient may be treated according to the following regimen. One of olaparib, niraparib, rucaparib or talazoparib (PARPi) is orally administered according to one of the dosing regimens listed in Example 2, accompanied by intravenous administration of a radioconjugated NKG2DL targeting agent as detailed herein in either single or fractional administration. For example, the dosing regimens include, by way of example: (a) the PARPi and the radioconjugated NKG2DL targeting agent administered concurrently, wherein (i) each is administered beginning on the same day, (ii) the radioconjugated NKG2DL targeting agent is administered in a single dose or fractionated doses not less than one week apart, and (iii) the PARPi is administered daily or twice daily (as appropriate), and for a duration equal to or exceeding that of the radioconjugated NKG2DL targeting agent administration; or (b) the PARPi and radioconjugated NKG2DL targeting agent are administered concurrently, wherein (i) the PARPi administration precedes radioconjugated NKG2DL targeting agent administration by at least one week, (ii) the radioconjugated NKG2DL targeting agent is administered in a single dose or fractionated doses not less than one week apart, and (iii) the PARPi is administered daily or twice daily (as appropriate), and for a duration equal to or exceeding that of the radioconjugated NKG2DL targeting agent administration.

### Example 8: Dosing Regimens for a NKG2DL Targeting Agent and a CD47 Blockade

The CD47 blocking agent may be a monoclonal antibody that prevents CD47 binding to SIRPα. Exemplary monoclonal antibodies include at least magrolimab, lemzoparlimab, AO-176, TTI-621, TTI-622, or a combination thereof the CD47 blockade may alternatively, or additionally, include agents that modulate the expression of CD47 and/or SIRPα, such as phosphorodiamidate morpholino oligomers (PMO) that block translation of CD47. Therapeutically effective doses of anti-CD47 antibodies include at least 0.05-10 mg/kg.

Thus, methods of the present disclosure may include administering one or more of the anti-CD47 antibodies or other agents, accompanied by intravenous administration of a radioconjugated NKG2DL targeting agent as detailed herein in either single or fractional administration. For example, the dosing regimens include, by way of example: (a) the anti-CD47 antibody or agent and the radioconjugated NKG2DL targeting agent administered concurrently, wherein (i) each is administered beginning on the same day, (ii) the radioconjugated NKG2DL targeting agent is administered in a single dose or fractionated doses not less than one week apart, and (iii) the anti-CD47 antibody or agent is administered daily or twice daily (as appropriate), and for a duration equal to or exceeding that of the radioconjugated NKG2DL targeting agent administration; or (b) the anti-CD47 antibody or agent and radioconjugated NKG2DL targeting agent are administered concurrently, wherein (i) the anti-CD47 antibody or agent administration precedes radioconjugated NKG2DL targeting agent administration by at least one week, (ii) the radioconjugated NKG2DL targeting agent is administered in a single dose or fractionated doses not less than one week apart, and (iii) the anti-CD47 antibody or agent is administered daily or twice daily (as appropriate), and for a duration equal to or exceeding that of the radioconjugated NKG2DL targeting agent administration.

### Example 9: Dosing Regimens for a NKG2DL Targeting Agent and an ICI

The immune checkpoint inhibitor (ICI) may be a monoclonal antibody against any of PD-1, PD-L1, PD-L2, CTLA-4, TIM3, LAG3 or VISTA. Therapeutically effective doses of these antibodies include at least 0.05-10 mg/kg. Thus, methods of the present disclosure include administering one or more ICI, accompanied by intravenous administration of a radioconjugated NKG2DL targeting agent as detailed herein in either single or fractional administration. For example, the dosing regimens include, by way of example: (a) the ICI and the radioconjugated NKG2DL targeting agent administered concurrently, wherein (i) each is administered beginning on the same day, (ii) the radioconjugated NKG2DL targeting agent is administered in a single dose or fractionated doses not less than one week apart, and (iii) the ICI is administered daily or twice daily (as appropriate), and for a duration equal to or exceeding that of the radioconjugated NKG2DL targeting agent administration; or (b) the ICI and radioconjugated NKG2DL targeting agent are administered concurrently, wherein (i) the anti-CD47 antibody administration precedes radioconjugated NKG2DL targeting agent administration by at least one week, (ii) the radioconjugated NKG2DL targeting agent is administered in a single dose or fractionated doses not less than one week apart, and (iii) the ICI is administered daily or twice daily (as appropriate), and for a duration equal to or exceeding that of the radioconjugated NKG2DL targeting agent administration.

Without limitation, the following aspects of the invention are also provided:

A method for treating a cancer in a mammalian subject such as a human, the method including: administering to the subject a therapeutically effective amount of a radioconjugated (radiolabeled) NKG2DL targeting agent.

The method according to any previous aspect, including before said administering step, diagnosing the subject with NKG2DL-positive cells; and if the subject has NKG2DL-positive cells, proceeding with administering to the subject the therapeutically effective amount of a NKG2DL targeting agent.

The method according to the previous aspect, wherein diagnosing includes obtaining a sample of tissue from the subject, mounting the sample on a substrate, and detecting the presence or absence of NKG2DL antigen using a diagnostic antibody, wherein the diagnostic antibody includes an antibody against NKG2DL labeled with a radiolabel such as ³H, ¹⁴C, ³²P, ³⁵S and ¹²⁵⁷I, fluorescent or chemiluminescent compounds, such as fluorescein isothiocyanate, rhodamine, or luciferin, or an enzyme, such as alkaline phosphatase, β-galactosidase, or horseradish peroxidase; or wherein diagnosing includes administering an NKG2DL targeting agent to the subject, wherein the NKG2DL targeting agent includes a radiolabel selected from the group including ¹⁸F, ¹¹C, ⁶⁸Ga, ⁶⁴Cu, ⁸⁹Zr, ¹²⁴I, ^{99m}Tc, or ¹¹¹In, waiting a time sufficient to allow the NKG2DL targeting agent to accumulate at a tissue site, and imaging the tissues with a non-invasive imaging technique to detect presence or absence of NKG2DL-positive cells, wherein the non-invasive imaging technique includes positron emission tomography (PET imaging) for ¹⁸F, ¹¹C, ⁶⁸Ga ⁶⁴Cu, ⁸⁹Zr, or ¹²⁴I labeled NKG2DL targeting agents or single photon emission computed tomography (SPECT imaging) for ^{99m}Tc or ¹¹¹In labeled NKG2DL targeting agents.

The method according to any preceding aspect, wherein the cancer is a solid cancer such as sarcoma, osteosarcoma, Ewing's sarcoma, a carcinoma, breast cancer, TNBC, gastric cancer, bladder cancer, cervical cancer, endometrial cancer, skin cancer, melanoma, stomach cancer, testicular cancer, esophageal cancer, bronchioloalveolar cancer, prostate cancer, CRPC, colorectal cancer, ovarian cancer, cervical epidermoid cancer, pancreatic cancer, lung cancer, NSCLC, SCLC, renal cancer, head and neck cancer, head and neck squamous cell carcinoma, gastric cancer, ovarian cancer, HCC, cholangiocarcinoma, or any of the solid cancers or precancerous proliferative disorders disclosed herein.

The method according to any preceding aspect, wherein the cancer includes/is a hematological cancer such as leukemia, acute leukemia, acute myeloid leukemia (AML), acute promyelocytic leukemia, chronic myelogenous leukemia (CML; a/k/a chronic myeloid leukemia), hairy cell leukemia, myelodysplastic syndrome (MDS), myeloproliferative neoplasms (MPNs), acute lymphoblastic leukemia (ALL), chronic lymphocytic leukemia (CLL), lymphoma, Hodgkin lymphoma, non-Hodgkin lymphoma, mantle cell lymphoma, diffuse large B-cell lymphoma (DLBCL), cutaneous T-cell lymphoma (CTCL), peripheral T-cell lymphoma (CTCL), Burkitt lymphoma, follicular lymphoma, high-grade B-cell lymphoma, Waldenstrom's macroglobulinaemia (WM), or multiple myeloma.

The method according to any preceding aspect, wherein the radioconjugated NKG2DL targeting agent includes a radiolabel selected from ¹³¹I, ¹²⁵I, ¹²³I, ⁹⁰Y, ¹⁷⁷Lu, ¹⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or any combination thereof and, for example, the radioconjugated NKG2DL targeting agent is for therapeutic use.

The method according to any preceding aspect, wherein the radioconjugated NKG2DL targeting agent includes a radiolabel selected from ¹³¹I, ⁹⁰Y, ¹⁷⁷Lu, ²²⁵Ac, ²¹³Bi, ²¹¹At, ²²⁷Th, or ²¹²Pb, or any combination thereof, and, for example, the radioconjugated NKG2DL targeting agent is for therapeutic use.

The method according to any preceding aspect, wherein the radioconjugated NKG2DL targeting agent is ²²⁵Ac-, ¹⁷⁷Lu-, or ¹³¹I-labeled.

The method according to any preceding aspect, wherein the effective amount of the radioconjugated NKG2DL targeting agent is a maximum tolerated dose (MTD) or a minimum effective dose (MED).

The method according to any preceding aspect, wherein the radioconjugated NKG2DL targeting agent is a recombinant soluble NKG2D receptor, a monoclonal antibody, antigen-binding antibody fragment, small protein, peptide, or small molecule against (that binds) one or more NKG2DL, such as one or more of MICA, MICB, ULBP1, ULBP2, ULBP3, ULBP4, ULBP5, and ULBP6, such as human forms thereof.

The method according to any preceding aspect, wherein the radioconjugated NKG2DL targeting agent binds to MICA and optionally also to MICB and/or ULBP1, such as to the human forms of these proteins.

The method according to any preceding aspect, wherein the radioconjugated NKG2DL targeting agent binds the alpha-3 domain of MICA, and/or blocks cleavage at the alpha-3 domain of MICA.

The method according to any preceding aspect, wherein the therapeutically effective amount of the radioconjugated NKG2DL targeting agent includes a single dose that delivers less than 2Gy, or less than 8 Gy, such as doses of 2 Gy to 8 Gy, to the subject.

The method according to any preceding aspect, wherein the radioconjugated NKG2DL targeting agent is ²²⁵Ac-labeled, and the effective amount of the ²²⁵Ac-labeled NKG2DL targeting agent includes a dose of 0.1 to 50 μCi/kg body weight of the subject, or 0.2 to 20 μCi/kg body weight of the subject, or 0.5 to 10 μCi/kg subject body weight.

The method according to any preceding aspect, wherein the radioconjugated NKG2DL targeting agent is a recombinant soluble NKG2D receptor, such as a homodimeric NKG2D-Fc fusion protein, or full-length antibody against NKG2DL that is ²²⁵Ac-labeled, and the effective of the ²²⁵Ac-labeled NKG2DL targeting agent includes less than 5 μCi/kg body weight of the subject, such as 0.1 to 5 μCi/kg body weight of the subject.

The method according to any preceding aspect, wherein the radioconjugated NKG2DL targeting agent is an antibody fragment, such an Fab or Fab2 fragment, or an scFv molecule, or a minibody or a nanobody against an NKG2DL that is ²²⁵Ac-labeled, and the effective of the ²²⁵Ac-labeled NKG2DL targeting agent includes greater than 5 μCi/kg body weight of the subject, such as 5 to 20 μCi/kg body weight of the subject.

The method according to any preceding aspect, wherein the radioconjugated NKG2DL targeting agent is ²²⁵Ac-labeled, and the effective amount of the ²²⁵Ac-labeled NKG2DL targeting agent includes 2 μCi to 2 mCi, or 2 μCi to 250 μCi, or 75 μCi to 400 μCi.

The method according to any preceding aspect, wherein the radioisotope labeled NKG2DL targeting agent is ¹⁷⁷Lu-labeled and the effective amount of the NKG2DL targeting agent includes a dose of less than 1000 μCi/kg body weight of the subject, such as a dose of 1 to 900 μCi/kg body weight of the subject, or 5 to 250 μCi/kg body weight of the subject or 50 to 450 μCi/kg body weight.

The method according to any preceding aspect, wherein the radioisotope labeled NKG2DL targeting agent is ¹⁷⁷Lu-labeled, and the effective amount of the ¹⁷⁷Lu-labeled NKG2DL targeting agent includes a dose of 10 mCi to at or below 30 mCi, or from at least 100 μCi to at or below 3 mCi, or from 3 mCi to at or below 30 mCi.

The method according to any preceding aspect, wherein the radioconjugated NKG2DL targeting agent is ¹³¹I-labeled, and the effective amount of the ¹³¹I-labeled NKG2DL targeting agent includes a dose of less than 1200 mCi, such as a dose of 25 to 1200 mCi, or 100 to 400 mCi, or 300 to 600 mCi, or 500 to 1000 mCi.

The method according to any preceding aspect, wherein the radioconjugated NKG2DL targeting agent is ¹³¹I-labeled, and the effective amount of the ¹³¹I-labeled NKG2DL targeting agent includes a dose of less than 200 mCi, such as a dose of 1 to 200 mCi, or 25 to 175 mCi, or 50 to 150 mCi.

The method according to any preceding aspect, wherein the effective amount of the NKG2DL targeting agent includes a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

The method according to any preceding aspect, wherein the therapeutically effective amount of the radioconjugated NKG2DL targeting agent is an amount effective to deplete or ablate NKG2DL-positive cells in a solid tumor or in a solid tumor cancer metastasis or in a solid tumor precancerous lesion, or of a hematological malignancy, independent of ADCC and optionally the depletion or ablation is not mediated by ADCC.

The method according to any preceding aspect, wherein the therapeutically effective amount of the radioconjugated NKG2DL targeting agent is an amount at least 10-fold lower than an unconjugated NKG2DL targeting agent, or an amount at least 20-fold lower than the unconjugated NKG2DL targeting agent, or an amount at least 30-fold lower than the unconjugated NKG2DL targeting agent.

The method according to any preceding aspect, wherein the cancer includes a solid tumor and the therapeutically effective amount of the radioisotope labeled NKG2DL targeting agent is an amount effective to increase expression of NKG2DL on cells within a tumor, or the cancer includes a hematological cancer and the therapeutically effective amount of the radioisotope labeled NKG2DL targeting agent is an amount effective to increase expression of NKG2DL on the hematological cancer cells.

The method according to any preceding aspect, wherein the NKG2DL targeting agent is administered according to a dosing schedule selected from the group consisting of once every 7, 10, 12, 14, 20, 24, 28, 36, and 42 days throughout a treatment period, wherein the treatment period includes at least two doses.

The method according to any preceding aspect, wherein the NKG2DL targeting agent is a peptide or small molecule.

The method according to any preceding aspect, wherein the NKG2DL targeting agent is an antibody mimetic protein, DARPin, anticalin, affimer, or aptamer.

The method according to any preceding aspect, further including administering to the subject a therapeutically effective amount of an immune checkpoint therapy, a chemotherapeutic agent, a DNA damage response inhibitor (DDRi), a targeting agent against a non-NGK2DL cancer associated antigen (e.g., radiolabeled, drug-conjugated, or naked with therapeutic activity), a CD47 blockade, an HDAC inhibitor, an LSD1 inhibitor, an adoptive cell therapy, or any combination thereof.

The method according to any preceding aspect, wherein the immune checkpoint therapy includes an antibody or small molecule inhibitor directed to CTLA-4, PD-1, TIM-3, VISTA, BTLA, LAG-3, TIGIT, A2aR, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or any combination thereof.

The method according to any preceding aspect, wherein the immune checkpoint therapy includes an antibody or small molecule inhibitor directed to PD-1, PD-L1, PD-L2, CTLA-4, CD137, A2aR, or any combination thereof.

The method according to any preceding aspect, wherein the DDRi includes a poly(ADP-ribose) polymerase inhibitor (PARPi), an ataxia telangiectasia mutated inhibitor (ATMi), an ataxia talangiectasia mutated and Rad-3 related inhibitor (ATRi), or a Wee1 inhibitor.

The method according to any preceding aspect, wherein the PARPi includes one or more of olaparib, niraparib, rucaparib and talazoparib.

The method according to any preceding aspect, wherein the ATMi includes one or more of KU-55933, KU-59403, wortmannin, CP466722, or KU-60019.

The method according to any preceding aspect, wherein the ATRi includes one or more of Schisandrin B, NU6027, NVP-BEA235, VE-821, VE-822, AZ20, or AZD6738.

The method according to any preceding aspect, wherein the Wee1 inhibitor includes AZD-1775 (i.e., adavosertib).

The method according to any preceding aspect, wherein the CD47 blockade includes: a monoclonal antibody against CD47, and/or a monoclonal antibody against SIRPα; and/or a SIRPα-Fc fusion protein that prevents CD47 binding to SIRPα; and/or an agent that modulates CD47 expression such as MBT-001; and/or a small molecule inhibitor such as RRx-001 or a pharmaceutically acceptable salt thereof.

The method according to any preceding aspect, wherein the CD47 blockade includes one or more of magrolimab, lemzoparlimab, AO-176, TTI-621, TTI-622, RRx-001 or a pharmaceutically acceptable salt thereof, an agent that modulates CD47 expression includes phosphorodiamidate morpholino oligomers (PMO) that block translation of CD47 such as MBT-001, or any combination thereof

The method according to any preceding aspect, wherein the therapeutically effective amount of the CD47 blockade includes 0.05 to 5 mg/Kg patient weight.

The method according to any preceding aspect, wherein the NKG2DL targeting agent is administered before an immune checkpoint therapy, DDRi, or CD47 blockade; or wherein an immune checkpoint therapy, DDRi, or CD47 blockade administered before the NKG2DL targeting agent.

The method according to any preceding aspect, wherein the NKG2DL targeting agent is administered with one of the immune checkpoint therapy or the DDRi or the CD47 blockade, and the others of the immune checkpoint therapy or the DDRi or the CD47 blockade are administered either before or after the NKG2DL targeting agent.

The method according to any preceding aspect, wherein the NKG2DL targeting agent is administered simultaneously with the immune checkpoint therapy and/or the DDRi and/or the CD47 blockade.

The method according to any preceding aspect, wherein the NKG2DL targeting agent is a multispecific antibody, wherein the multispecific antibody includes: a first target recognition component that specifically binds to an epitope of a NKG2DL, and a second target recognition component that specifically binds to a different epitope of the NKG2DL than the first target recognition component, or to a different NKG2DL, or to an epitope of a different, non-NKG2DL antigen such as a cancer associated antigen, such as any of those disclosed herein.

Another aspect of the invention provides a therapeutic composition for the treatment of cancer in a mammalian subject such as a human, the composition including: an 22′Ac-labeled NKG2DL targeting agent, such as an ²²⁵Ac-labeled NKG2DL antibody or recombinant soluble NKG2D receptor (such as an NKG2D-Fc fusion protein), provided in a patient specific dose, and a pharmaceutically acceptable carrier, wherein the patient specific dose includes a protein dose of 0.001 to 3.0 mg/kg subject body weight, and a radiation dose of 0.1 to 50 μCi/kg subject body weight, wherein each of the protein dose and the radiation dose are selected based on patient specific characteristics including any one or more of a patient weight, gender, age, or health status.

Another aspect provides the therapeutic composition according to the previous aspect, wherein the protein dose is from 0.01 to 1 mg/kg subject body weight, and the radiation dose is from 0.1 to 5 μCi/kg subject body weight, or 5 to 20 μCi/kg subject body weight; or wherein the protein dose is from 0.01 to 1 mg/kg subject body weight, and the radiation dose is from 2 μCi to 2 mCi, or 2 μCi to 250 μCi, or 75 μCi to 400 μCi.

Another aspect provides the therapeutic composition according to any previous composition aspect, wherein the NKG2DL targeting agent includes a monoclonal antibody against MICA, such as a monoclonal antibody against MICA targets an epitope on alpha-3 domain of MICA, or blocks cleavage at the alpha-3 domain of MICA.

The NKG2D targeting agent that is radiolabeled in any of the preceding aspects, may for example, include a monoclonal antibody against MICA (such as against human MICA), such as any of those disclosed herein, such as clones 1D5, 14B4, 16A8. The radiolabeled NKG2DL targeting agent for use in any of the aspects of the invention may, for example, be an anti-MICA antibody that recognizes human MICA, which antibody includes:
(a) CDR-L1 (SEQ ID NO:1), CDR-L2 (SEQ ID NO:2), and CDR-L3 (SEQ ID NO: 3) and/or CDR-H1 (SEQ ID NO:4), CDR-H2 (SEQ ID NO:5), and CDR-H3 (SEQ ID NO:6);
(b) immunoglobulin light chain variable region (SEQ ID NO:7) and/or immunoglobulin heavy chain variable region (SEQ ID NO:8);
(c) immunoglobulin light chain variable region (SEQ ID NO:9) and/or immunoglobulin heavy chain variable region (SEQ ID NO:10);
(d) CDR-L1 (SEQ ID NO:11), CDR-L2 (SEQ ID NO:12), and CDR-L3 (SEQ ID NO:13) and/or CDR-H1 (SEQ ID NO:14), CDR-H2 (SEQ ID NO:15), and CDR-H3 (SEQ ID NO:16);
(e) immunoglobulin light chain variable region (SEQ ID NO:17) and/or immunoglobulin heavy chain variable region (SEQ ID NO:18);
(f) an scFv molecule including SEQ ID NO: 19;
(g) CDR-L1 (SEQ ID NO:20), CDR-L2 (SEQ ID NO:21), and CDR-L3 (SEQ ID NO:22) and/or CDR-H1 (SEQ ID NO:23, 24 or 25), CDR-H2 (SEQ ID NO:26 or 27), and CDR-H3 (SEQ ID NO:28);
(h) immunoglobulin light chain variable region (SEQ ID NO:29) and/or immunoglobulin heavy chain variable region (SEQ ID NO:30);
(i) CDR-L1 (SEQ ID NO:31), CDR-L2 (SEQ ID NO:32), and CDR-L3 (SEQ ID NO:33) and/or CDR-H1 (SEQ ID NO:34, 35 or 36), CDR-H2 (SEQ ID NO:37 or 38), and CDR-H3 (SEQ ID NO:39);
(j) immunoglobulin light chain variable region (SEQ ID NO:40) and/or immunoglobulin heavy chain variable region (SEQ ID NO:41); or
(k) CDR-L1 containing sequence (SEQ ID NO:42), CDR-L2 containing sequence (SEQ ID NO:43), and CDR-L3 containing sequence (SEQ ID NO:44) and/or CDR-H1 containing sequence (SEQ ID NO:45), CDR-H2 containing sequence (SEQ ID NO:46) and CDR-H3 containing sequence (SEQ ID NO:47), wherein light chain CDRs are designated CDR-L1-3 and heavy chain CDRs are designated CDR-H1-3.

Any and all publications, patents, patent applications and other documents cited in this application are hereby incorporated by reference in their entireties for all purposes to the same extent as if each individual publication, patent, patent application or other document were individually indicated to be incorporated by reference for all purposes.

It should be understood that wherever in this disclosure an aspect or embodiment of the invention or an element or step thereof is described in terms of “including,” “include(s),” “comprising,” or “comprise(s),” corresponding aspects, embodiments, elements or steps thereof expressed, instead, in terms of “consisting essentially of” or “consisting of” are also intended to be disclosed and provided by this disclosure.

While various specific embodiments have been illustrated and described herein, it will be appreciated that various changes can be made without departing from the spirit and scope of the invention(s). Moreover, features described in connection with one aspect or embodiment of the invention may be used in conjunction with other aspects or embodiments, even if not explicitly exemplified in combination within.

### REFERENCES

Bagley S J, Desai A S, Linette G P, June C H, O'Rourke D M. CAR T cell therapy for glioblastoma: recent clinical advances and future challenges. Neuro Oncol. 2018; 20(11):1429-1438.
Barsheshet Y, Wildbaum G, Levy E, et al. NKG2DL+FOXp3+ Treg cells as master drivers of immune regulation. Proc Natl Acad Sci. 2017; 114(23):6086-6091.
Cao X, Cai S F, Fehniger T A, et al. Granzyme B and Perforin Are Important for Regulatory T Cell-Mediated Suppression of Tumor Clearance. Immunity. 2007; 27(4):635-646.
Couper K N, Lanthier P A, Perona-Wright G, et al. Anti-CD25 Antibody-Mediated Depletion of Effector T Cell Populations Enhances Susceptibility of Mice to Acute but Not Chronic Toxoplasma gondii Infection. J Immunol. 2009; 182(7):3985-3994.
Dar T B, Henson R M, Shiao S L. Targeting Innate Immunity to Enhance the Efficacy of Radiation Therapy. Front Immunol. 2019; 9(JAN):1-11.
Dawicki W, et al. Daratumumab-225 Actinium conjugate demonstrates greatly enhanced antitumor activity against experimental multiple myeloma tumors. Oncoimmunology. 2019; 8(8):e1607673.
Dépis F, Hu C, Weaver J, et al. Abstract 4532: Preclinical evaluation of JTX-1811, an anti-NKG2D L antibody with enhanced ADCC activity, for preferential depletion of tumor-infiltrating regulatory T cells. In: Cancer Research. Vol 80. American Association for Cancer Research (AACR); 2020:4532-4532.
De Simone M, Arrigoni A, Rossetti G, et al. Transcriptional Landscape of Human Tissue Lymphocytes Unveils Uniqueness of Tumor-Infiltrating T Regulatory Cells. Immunity. 2016; 45(5):1135-1147.
Drago J Z, Modi S, Chandarlapaty S. Unlocking the potential of antibody-drug conjugates for cancer therapy. Nat Rev Clin Oncol. Published online Feb. 8, 2021.
Epperly R, Gottschalk S, Velasquez M P. A Bump in the Road: How the Hostile AML Microenvironment Affects CAR T Cell Therapy. Front Oncol. 2020; 10:262.
Eyquem, et al. Targeting a CAR to the TRAC locus with CRISPR/Cas9 enhances tumour rejection. Nature. 2017, 543:113-117.
Fridman W H, Pages F, Sauis-Fridman C, Galon J. The immune contexture in human tumours: Impact on clinical outcome. Nat Rev Cancer. 2012; 12(4):298-306.
Han G, et al. Tim-3: an activation marker and activation limiter of innate immune cells. Front Immunol. 2013; 4:449.
Haberkorn U, Giesel F, Morgenstern A, Kratochwil C. The future of radioligand therapy: α, β, or Both? J Nucl Med. 2017; 58(7):1017-1018.
Hellmann M D, Ciuleanu T-E, Pluzanski A, et al. Nivolumab plus Ipilimumab in Lung Cancer with a High Tumor Mutational Burden. N Engl J Med. 2018; 378(22):2093-2104.
Hoelzinger D B, Smith S E, Mirza N, Dominguez A L, Manrique S Z, Lustgarten J. Blockade of CCL1 Inhibits T Regulatory Cell Suppressive Function Enhancing Tumor Immunity without Affecting T Effector Responses. J Immunol. 2010; 184(12):6833-6842.
Kawashima A, Kanazawa T, Yoshida T, et al. MP18-02 identification of NKG2D L as a specific marker of tumor tissue-infiltrating regulatory t cells and its possibility as a therapeutic target in renal cell carcinoma. J Urol. 2020; 203:e234.
Lake A, Warren M, Das S, et al. 726 SRF114 is a fully human, NKG2D L selective IgG1 antibody that induces destruction of tumor Tregs through ADCC. J Immunother Cancer. 2020; 8(Suppl 3):A769-A769.
Lamichhane P, Amin N, Agarwal M, Lamichhane N. Checkpoint Inhibition: Will Combination with Radiotherapy and Nanoparticle-Mediated Delivery Improve Efficacy? Medicines. 2018; 5, 114.
Lammermann T, Kastenmuller W. Concepts of GPCR—controlled navigation in the immune system. Immunol Rev. 2019; 289(1):205-231.
Lan R, Jhatakia A, Campbell J, et al. Abstract 6694: Highly selective anti-NKG2D L antibody-mediated depletion of regulatory T cells leads to potent antitumor activity alone and in combination with anti-PD-1 in preclinical models. In: Cancer Research. Vol 80. American Association for Cancer Research (AACR); 2020:6694-6694.
Lu S, Hu S, Gan X, et al. 711 HBM1022, a novel anti-NKG2D L antibody depletes tumor-infiltrating regulatory T cells via enhanced ADCC activity, mediates potent anti-tumor activity with Keytruda. J Immunother Cancer. 2020; 8(Suppl 3):A753-A753.
Magee M S, Kraft C L, Abraham T S, et al. GUCY2C-directed CAR-T cells oppose colorectal cancer metastases without autoimmunity. Oncoimmunology. 2016; 5(10):e1227897.
Magnuson A M, Kiner E, Ergun A, et al. Identification and validation of a tumor-infiltrating Treg transcriptional signature conserved across species and tumor types. Proc Natl Acad Sci USA. 2018; 115(45):E10672-E10681.
Maleki Vareki S, Garrigós C, Duran I. Biomarkers of response to PD-1/P D-L1 inhibition. Crit Rev Oncol Hematol. 2017; 116:116-124.
Marabelle A, Kohrt H, Sagiv-Barfi I, et al. Depleting tumor-specific Tregs at a single site eradicates disseminated tumors. J Clin Invest. 2013; 123(6):2447-2463.
Mardiana S, Solomon B J, Darcy P K, Beavis P A. Supercharging adoptive T cell therapy to overcome solid tumor-induced immunosuppression. Sci Transl Med. 2019; 11(495):2293.
Melaiu O, Lucarini V, Cifaldi L, Fruci D. Influence of the Tumor Microenvironment on N K Cell Function in Solid Tumors. Front Immunol. 2020; 10:3038.
Nelson B, Andersson J, Wuest F. Targeted Alpha Therapy: Progress in Radionuclide Production, Radiochemistry, and Applications. Pharmaceutics. 2020; 13(1):49.
Neti P V S V, Howell R W. Log normal distribution of cellular uptake of radioactivity: implications for biologic responses to radiopharmaceuticals. J Nucl Med. 2006; 47(6):1049-1058.
Nishikawa H, Sakaguchi S. Regulatory T cells in tumor immunity. Int J Cancer. 2010; 127(4):759-767.
Ogbomo H, Cinatl J, Mody C H, Forsyth P A. Immunotherapy in gliomas: Limitations and potential of natural killer (N K) cell therapy. Trends Mol Med. 2011; 17(8):433-441.
Ohue Y, Nishikawa H. Regulatory T (Treg) cells in cancer: Can Treg cells be a new therapeutic target? Cancer Sci. 2019; 110(7):2080-2089.
Onizuka S, Tawara I, Shimizu J, Sakaguchi S, Fujita T, Nakayama E. Tumor rejection by in vivo administration of anti-CD25 (interleukin-2 receptor a) monoclonal antibody. Cancer Res. 1999; 59(13):3128-3133.
Papazahariadou, et al. Involvement of N K cells against tumors and parasites. Int. J. Biol. Markers. 2007; 22:144-53.
Paschen, et al. Differential Clinical Significance of Individual NKG2D Ligands in Melanoma: Soluble ULBP2 as an Indicator of Poor Prognosis Superior to S100B. Clin. Cancer Res. 2009; 15:5208-15.
Peggs K S, Quezada S A, Chambers C A, Korman A J, Allison J P. Blockade of CTLA-4 on both effector and regulatory T cell compartments contributes to the antitumor activity of anti-CTLA-4 antibodies. J Exp Med. 2009; 206(8):1717-1725.
Plitas G, Konopacki C, Wu K, et al. Regulatory T Cells Exhibit Distinct Features in Human Breast Cancer. Immunity. 2016; 45(5):1122-1134.
Rankin A, Naik E. 861 Development of FPA157, an anti-NKG2D L depleting antibody engineered to preferentially eliminate tumor-infiltrating T regulatory cells. J Immunother Cancer. 2020; 8(Suppl 3):A914-A914.
Ren, et. al. Multiplex Genome Editing to Generate Universal CART Cells Resistant to PD1 Inhibition. Clin. Cancer Res. 2017, 23:2255-2266.
Saleh R, Elkord E. FoxP3+T regulatory cells in cancer: Prognostic biomarkers and therapeutic targets. Cancer Lett. 2020; 490:174-185.
Santoni, et al. Natural Killer (N K) Cells from Killers to Regulators: Distinct Features Between Peripheral Blood and Decidual N K Cells. Am. J. Reprod Immunol. 2007; 58:280-88.
Sharabi A B, Lim M, DeWeese T L, Drake C G. Radiation and checkpoint blockade immunotherapy: radiosensitisation and potential mechanisms of synergy. Lancet Oncol. 2015; 16(13):e498-509.
Shimizu J, Yamazaki S, Sakaguchi S. Induction of tumor immunity by removing CD25+CD4+ T cells: a common basis between tumor immunity and autoimmunity, J. Immunol 1999; 163(10):5211-8.
Simpson T R, Li F, Montalvo-Ortiz W, et al. Fc-dependent depletion of tumor-infiltrating regulatory t cells co-defines the efficacy of anti-CTLA-4 therapy against melanoma. J Exp Med. 2013; 210(9):1695-1710.
Takimoto C H, Chao M P, Gibbs C, et al. The Macrophage “Do not eat me” signal, CD47, is a clinically validated cancer immunotherapy target. Ann Oncol. 2019; 30(3):486-489.
Tanaka A, Sakaguchi S. Targeting Treg cells in cancer immunotherapy. Eur J Immunol. 2019; 49(8):eji.201847659.
Twyman-Saint Victor C et al. Radiation and dual checkpoint blockade activate non-redundant immune mechanisms in cancer. Nature. 2015; 520:373-377.
Van Damme H, Dombrecht B, Kiss M, et al. Therapeutic depletion of NKG2DL+tumor-infiltrating regulatory T cells elicits antitumor immunity and synergizes with anti-PD-1 therapy. J Immunother Cancer. 2021; 9(2):e001749.
Vignali D A A, Collison L W, Workman C J. How regulatory T cells work. Nat Rev Immunol. 2008; 8(7):523-532.
Villarreal D O, L'Huillier A, Armington S, et al. Targeting NKG2DL induces protective antitumor immunity and enhances vaccine-induced responses in colon cancer. Cancer Res. 2018; 78(18):5340-5348.
Wang Z, Chen W, Zhang X, Cai Z, Huang W. A long way to the battlefront: CAR T cell therapy against solid cancers. J Cancer. 2019; 10(14):3112-3123.
Wang Y, Liu Z-G, Yuan H, et al. The Reciprocity between Radiotherapy and Cancer Immunotherapy. Clin Cancer Res. 2019; 25(6):1709-1717.
Zheng C, Zheng L, Yoo J K, et al. Landscape of Infiltrating T Cells in Liver Cancer Revealed by Single-Cell Sequencing. Cell. 2017; 169(7):1342-1356.e16.

1. A method for treating a cancer in a mammalian subject, the method comprising:
administering to the subject a therapeutically effective amount of a radioconjugated NKG2DL targeting agent,
wherein the NKG2DL targeting agent binds one or more of MICA, MICB, ULBP1, ULBP2, ULBP3, ULBP4, ULBP5, and ULBP6, and
wherein the radioconjugated NKG2DL targeting agent comprises a radionuclide label selected from ²¹⁵Ac, ¹⁷⁷Lu, ⁹⁰Y, ²¹³Bi, ²¹¹At, ²²⁷Th, ²¹²Pb, or any combination thereof,
wherein the radioconjugated NKG2DL targeting agent comprises a radioconjugated anti-NKG2DL monoclonal antibody, a radioconjugated NKG2DL-binding fragment of a monoclonal antibody, or a soluble, recombinant NK2GD receptor,
wherein the radioconjugated NKG2DL targeting agent comprises a chemically conjugated chelator and the chelator chelates the radionuclide label,
wherein the cancer is a solid or hematological cancer, and
wherein the therapeutically effective amount of the radioconjugated NKG2DL targeting agent is an amount effective to deplete or ablate NKG2DL-positive solid cancer or hematological cancer cells independent of depletion or ablation mediated by ADCC.

2. The method of claim 1, wherein the solid or hematological cancer is selected from a carcinoma, a sarcoma, osteosarcoma, Ewing's sarcoma, breast cancer, TNBC, gastric cancer, bladder cancer, cervical cancer, endometrial cancer, skin cancer, stomach cancer, testicular cancer, esophageal cancer, bronchioloalveolar cancer, prostate cancer, castration-resistant prostate cancer, hepatocellular carcinoma, cholangiocarcinoma, colorectal cancer, ovarian cancer, cervical epidermoid cancer, pancreatic cancer, lung cancer, NSCLC, SCLC, renal cancer, head and neck cancer, leukemia, acute myeloid leukemia, chronic myeloid leukemia, acute lymphoblastic leukemia, lymphoma, Hodgkin lymphoma, non-Hodgkin lymphoma, multiple myeloma, or any combination thereof.

3. (canceled)

4. The method of claim 1, wherein the therapeutically effective amount of the radioconjugated NKG2DL targeting agent is an amount at least 10-fold lower than an unconjugated NKG2DL targeting agent, or an amount at least 20-fold lower than the unconjugated NKG2DL targeting agent, or an amount at least 30-fold lower than the unconjugated NKG2DL targeting agent.

5. The method of claim 1, wherein the cancer comprises a solid tumor and the therapeutically effective amount of the radioisotope labeled NKG2DL targeting agent is an amount effective to increase expression of NKG2DL on solid cancer or hematological cancer cells.

6. The method of claim 1, wherein the radioconjugated NKG2DL targeting agent binds to MICA.

7. The method of claim 6, wherein the NKG2DL targeting agent binds an epitope of MICA on an alpha-3 domain and/or blocks cleavage at the alpha-3 domain of MICA.

8. The method of claim 1, wherein the radioconjugated NKG2DL targeting agent comprises a soluble, recombinant NKG2D receptor protein.

9. The method of claim 1, wherein the radioconjugated NKG2DL targeting agent is an ²²⁵Ac-labeled NKG2DL targeting agent, and the therapeutically effective amount of the ²²⁵Ac-labeled NKG2DL targeting agent comprises:
a protein dose of less than 3 mg/kg subject body weight, such as from 0.001 mg/subject body weight to 3.0 mg/kg subject body weight, or from 0.005 mg/kg subject body weight to 2.0 mg/kg subject body weight, or from 0.01 mg/kg subject body weight to 1 mg/kg subject body weight, or from 0.1 mg/kg subject body weight to 0.6 mg/kg subject body weight, or 0.3 mg/kg subject body weight, or 0.4 mg/kg subject body weight, or 0.5 mg/kg subject body weight, or 0.6 mg/kg subject body weight; and
a radiation dose of 0.1 to 50 μCi/kg subject body weight, or 0.1 to 5 μCi/kg subject body weight, or 5 to 20 μCi/kg subject body weight.

10. The method of claim 1, wherein the radioconjugated NKG2DL targeting agent is an ²²⁵Ac-labeled NKG2DL targeting agent, and the therapeutically effective amount of the ²²⁵Ac-labeled NKG2DL targeting agent comprises:
a protein dose of less than 3 mg/kg subject body weight, such as from 0.001 mg/kg patient weight to 3.0 mg/kg subject body weight, or from 0.005 mg/kg subject body weight to 2.0 mg/kg subject body weight, or from 0.01 mg/kg subject body weight to 1 mg/kg subject body weight, or from 0.1 mg/kg subject body weight to 0.6 mg/kg subject body weight, or 0.3 mg/kg subject body weight, or 0.4 mg/kg subject body weight, or 0.5 mg/kg subject body weight, or 0.6 mg/kg subject body weight; and
a radiation dose of 2 μCi to 2 mCi, or 2 μCi to 250 μCi, or 75 μCi to 400 μCi.

11. The method of claim 1, wherein the therapeutically effective amount of the radioconjugated NKG2DL targeting agent is administered as a single dose.

12. The method of claim 1, wherein the radioconjugated NKG2DL targeting agent is administered according to a dosing schedule selected from the group consisting of once every 7, 10, 12, 14, 20, 24, 28, 36, and 42 days throughout a treatment period, wherein the treatment period includes at least two doses.

13. The method of claim 1, further comprising:
administering to the subject one or both of a therapeutically effective amount of an immune checkpoint therapy and a therapeutically effective amount of CD47 blockade.

14. The method of claim 13, wherein the immune checkpoint therapy comprises an antibody or small molecule inhibitor directed against PD-1, PD-L1, PD-L2, CTLA-4, TIM3, LAG3, VISTA, A2aR, or any combination thereof.

15. The method of claim 13, wherein the CD47 blockade comprises one or more of an anti-CD47 antibody, an anti-SIRPα antibody, a SIRPα-Fc fusion protein, magrolimab, lemzoparlimab, AO-176, TTI-621, TTI-622, ALX-148, RRx-001 and MBT-001.

16. (canceled)

17. (canceled)

18. (canceled)

19. The method of claim 1, further comprising, before administering the therapeutically effective amount of the radioconjugated NKG2DL targeting agent:
diagnosing the subject with NKG2DL-positive cells; and
if the subject has NKG2DL-positive cells, proceeding with administering to the subject the therapeutically effective amount of a NKG2DL targeting agent.

20. The method of claim 19, wherein the diagnosing comprises:
administering an NKG2DL targeting agent to the subject, wherein the NKG2DL targeting agent comprises a radiolabel selected from the group comprising ¹⁸F, ¹¹C, ⁶⁸Ga ⁶⁴Cu, ⁸⁹Zr, ¹²⁴I, ^{99m}Tc, or ¹¹¹In;
after a time sufficient for the administered NKG2DL targeting agent to accumulate at a tissue site, imaging the tissues with a non-invasive imaging technique to detect presence or absence of NKG2DL-positive cells,
wherein the non-invasive imaging technique comprises positron emission tomography (PET imaging) for ¹⁸F, ¹¹C, ⁶⁸Ga, ⁶⁴Cu, ⁸⁹Zr, or ¹²⁴I labeled NKG2DL targeting agents or single photon emission computed tomography (SPECT imaging) for ^{99m}Tc or ¹¹¹In labeled NKG2DL targeting agents.

21. (canceled)

22. (canceled)

23. The method of claim 1, wherein the chelator comprises DOTA or a DOTA derivative.

24. The method of claim 23, wherein the radioconjugated NKG2DL targeting agent is the product of reacting the NKG2DL targeting agent with p-SCN-Bn-DOTA and chelating a radionuclide to the DOTA moiety.

25. The method of claim 1, wherein the radioconjugated NKG2D targeting agent is a radiolabeled anti-(human MICA) antibody comprising:
(a) CDR-L1 (SEQ ID NO:1), CDR-L2 (SEQ ID NO:2), and CDR-L3 (SEQ ID NO:3) and CDR-H1 (SEQ ID NO:4), CDR-H2 (SEQ ID NO:5), and CDR-H3 (SEQ ID NO:6);
(b) immunoglobulin light chain variable region (SEQ ID NO:7) and immunoglobulin heavy chain variable region (SEQ ID NO:8);
(c) immunoglobulin light chain variable region (SEQ ID NO:9) and immunoglobulin heavy chain variable region (SEQ ID NO:10);
(d) CDR-L1 (SEQ ID NO:11), CDR-L2 (SEQ ID NO:12), and CDR-L3 (SEQ ID NO: 13) and CDR-H1 (SEQ ID NO:14), CDR-H2 (SEQ ID NO:15), and CDR-H3 (SEQ ID NO:16);
(e) immunoglobulin light chain variable region (SEQ ID NO:17) and immunoglobulin heavy chain variable region (SEQ ID NO:18);
(f) an scFv molecule including SEQ ID NO: 19;
(g) CDR-L1 (SEQ ID NO:20), CDR-L2 (SEQ ID NO:21), and CDR-L3 (SEQ ID NO:22) and CDR-H1 (SEQ ID NO:23, 24 or 25), CDR-H2 (SEQ ID NO:26 or 27), and CDR-H3 (SEQ ID NO:28);
(h) immunoglobulin light chain variable region (SEQ ID NO:29) and immunoglobulin heavy chain variable region (SEQ ID NO:30);
(i) CDR-L1 (SEQ ID NO:31), CDR-L2 (SEQ ID NO:32), and CDR-L3 (SEQ ID NO:33) and CDR-H1 (SEQ ID NO:34, 35 or 36), CDR-H2 (SEQ ID NO:37 or 38), and CDR-H3 (SEQ ID NO:39);
(j) immunoglobulin light chain variable region (SEQ ID NO:40) and immunoglobulin heavy chain variable region (SEQ ID NO:41); or
(k) CDR-L1 containing sequence (SEQ ID NO:42), CDR-L2 containing sequence (SEQ ID NO:43), and CDR-L3 containing sequence (SEQ ID NO:44) and CDR-H1 containing sequence (SEQ ID NO:45), CDR-H2 containing sequence (SEQ ID NO:46) and CDR-H3 containing sequence (SEQ ID NO:47).

26. The method of claim 1, wherein the mammalian subject is human.

27. A therapeutic composition for the treatment of an NKG2D-expressing solid cancer or hematological cancer in a mammalian subject, the composition comprising:
a therapeutically effective amount of a radionuclide-labeled NKG2DL targeting agent and
a pharmaceutically acceptable carrier,
wherein the radionuclide comprises one or more of ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, and ²¹²Pb, and
wherein the radioconjugated NKG2DL targeting agent comprises a radioconjugated anti-NKG2DL monoclonal antibody, a radioconjugated NKG2DL-binding fragment of a monoclonal antibody, or a soluble, recombinant NK2GD receptor.

28. The therapeutic composition of claim 27, wherein the radionuclide is ²²⁵Ac, the protein dose is from 0.01 to 1 mg/kg subject body weight, and the radiation dose is from 0.1 to 5 μCi/kg subject body weight, or 5 to 20 μCi/kg subject body weight.

29. The therapeutic composition of claim 27, wherein the radionuclide is ²²⁵Ac, the protein dose is from 0.01 to 1 mg/kg subject body weight, and the radiation dose is from 2 μCi to 2 mCi, or 2 μCi to 250 μCi, or 75 μCi to 400 μCi.

30. The therapeutic composition of claim 27, wherein the NKG2DL targeting agent binds to MICA.

31. The therapeutic composition of claim 30, wherein the NKG2DL targeting agent binds an epitope of MICA on an alpha-3 domain, or blocks cleavage at the alpha-3 domain of MICA.

32. (canceled)

33. The composition of claim 27, wherein the radioconjugated NKG2DL targeting agent is chemically conjugated to a chelator and the chelator chelates a radionuclide.

34. The composition of claim 33, wherein the chelator is DOTA or a DOTA derivative.

35. The composition of claim 34, wherein the radioconjugated NKG2DL targeting agent is the product of reacting the NKG2DL targeting agent with p-SCN-Bn-DOTA and chelating a radionuclide to the DOTA moiety.

36. (canceled)

37. (canceled)

38. The composition of claim 27, wherein the radiolabeled NKG2D targeting agent includes a radiolabeled anti-(human MICA) antibody comprising:
(a) CDR-L1 (SEQ ID NO: 1), CDR-L2 (SEQ ID NO:2), and CDR-L3 (SEQ ID NO:3) and CDR-H1 (SEQ ID NO:4), CDR-H2 (SEQ ID NO:5), and CDR-H3 (SEQ ID NO:6);
(b) immunoglobulin light chain variable region (SEQ ID NO:7) and immunoglobulin heavy chain variable region (SEQ ID NO:8);
(c) immunoglobulin light chain variable region (SEQ ID NO:9) and immunoglobulin heavy chain variable region (SEQ ID NO:10);
(d) CDR-L1 (SEQ ID NO:11), CDR-L2 (SEQ ID NO:12), and CDR-L3 (SEQ ID NO:13) and CDR-H1 (SEQ ID NO:14), CDR-H2 (SEQ ID NO:15), and CDR-H3 (SEQ ID NO:16);
(e) immunoglobulin light chain variable region (SEQ ID NO:17) and immunoglobulin heavy chain variable region (SEQ ID NO:18);
(f) an scFv molecule including SEQ ID NO: 19;
(g) CDR-L1 (SEQ ID NO:20), CDR-L2 (SEQ ID NO:21), and CDR-L3 (SEQ ID NO:22) and CDR-H1 (SEQ ID NO:23, 24 or 25), CDR-H2 (SEQ ID NO:26 or 27), and CDR-H3 (SEQ ID NO:28);
(h) immunoglobulin light chain variable region (SEQ ID NO:29) and immunoglobulin heavy chain variable region (SEQ ID NO:30);
(i) CDR-L1 (SEQ ID NO:31), CDR-L2 (SEQ ID NO:32), and CDR-L3 (SEQ ID NO:33) and CDR-H1 (SEQ ID NO:34, 35 or 36), CDR-H2 (SEQ ID NO:37 or 38), and CDR-H3 (SEQ ID NO:39);
(j) immunoglobulin light chain variable region (SEQ ID NO:40) and immunoglobulin heavy chain variable region (SEQ ID NO:41); or
(k) CDR-L1 containing sequence (SEQ ID NO:42), CDR-L2 containing sequence (SEQ ID NO:43), and CDR-L3 containing sequence (SEQ ID NO:44) and CDR-H1 containing sequence (SEQ ID NO:45), CDR-H2 containing sequence (SEQ ID NO:46) and CDR-H3 containing sequence (SEQ ID NO:47).

39. (canceled)


SEQUENCE LISTING (90 sequences)

SEQ ID NO: 1 - protein, 11 aa, Artificial Sequence
  Antibody component sequence
SASQDISNYLN

SEQ ID NO: 2 - protein, 7 aa, Artificial Sequence
  Antibody component sequence
DTSILHL

SEQ ID NO: 3 - protein, 9 aa, Artificial Sequence
  Antibody component sequence
QQYSKFPRT

SEQ ID NO: 4 - protein, 5 aa, Artificial Sequence
  Antibody component sequence
NYAMN

SEQ ID NO: 5 - protein, 17 aa, Artificial Sequence
  Antibody component sequence
WINTHTGDPTYADDFKG

SEQ ID NO: 6 - protein, 9 aa, Artificial Sequence
  Antibody component sequence
TYGNYAMDY

SEQ ID NO: 7 - protein, 107 aa, Artificial Sequence
  Antibody component sequence
DIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIK

SEQ ID NO: 8 - protein, 191 aa, Artificial Sequence
  Antibody component sequence
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSAKTTAPSVYPLAPVCGDTTGSSVTLGCLVKGYFPEPVTLTWNSGSLSSGVHTFPAVLQSDLYTLSSSVTVTSS

SEQ ID NO: 9 - protein, 109 aa, Artificial Sequence
  Antibody component sequence
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIKR

SEQ ID NO: 10 - protein, 120 aa, Artificial Sequence
  Antibody component sequence
  misc_feature (20)..(20): Xaa can be any naturally occurring amino acid
  misc_feature (35)..(35): Xaa can be any naturally occurring amino acid
QVQLVQSGAEVKKPGSSVRXSCRASGGSSTTYAFXWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 11 - protein, 11 aa, Artificial Sequence
  Antibody component sequence
QASQDIGNNLI

SEQ ID NO: 12 - protein, 7 aa, Artificial Sequence
  Antibody component sequence
YATNLAN

SEQ ID NO: 13 - protein, 7 aa, Artificial Sequence
  Antibody component sequence
QQWSSNP

SEQ ID NO: 14 - protein, 5 aa, Artificial Sequence
  Antibody component sequence
NYYMS

SEQ ID NO: 15 - protein, 17 aa, Artificial Sequence
  Antibody component sequence
NIYGGNGGTGYNQKFKG

SEQ ID NO: 16 - protein, 8 aa, Artificial Sequence
  Antibody component sequence
GDLYAMDY

SEQ ID NO: 17 - protein, 98 aa, Artificial Sequence
  Antibody component sequence
VLTQSPSSMSASLGDRVTITCQASQDIGNNLIWFQQKPGKSPRPMIYYATNLANGVPSRFSGSGSGTSYSLTISSMEAEDAATYYCQQWSSNPYTFGG

SEQ ID NO: 18 - protein, 106 aa, Artificial Sequence
  Antibody component sequence
AELVKPGASVKLSCKTSGYTFSNYYMSWLKQMPGQNIEWIGNIYGGNGGTGYNQKFKGKATLTVDKSSSTAYMQLSSLTSEDSAVYFCARGDLYAMDYWGQGTTVT

SEQ ID NO: 19 - protein, 245 aa, Artificial Sequence
  Antibody component sequence
MAQVQLQQSGAELVKPGASVKLSCKTSGYTFSNYYMSWLKQMPGQNIEWIGNIYGGNGGTGYNQKFKGKATLTVDKSSSTAYMQLSSLTSEDSAVYFCARGDLYAMDYWGQGTTVTVSSGGGGSGGGGSGGGGSDIVLTQSPSSMSASLGDRVTITCQASQDIGNNLIWFQQKPGKSPRPMIYYATNLANGVPSRFSGSGSGTSYSLTISSMEAEDAATYYCQQWSSNPYTFGGGTKLEIKRAAA

SEQ ID NO: 20 - protein, 11 aa, Artificial Sequence
  Antibody component sequence
RASQNIDTSIH

SEQ ID NO: 21 - protein, 7 aa, Artificial Sequence
  Antibody component sequence
YASESIS

SEQ ID NO: 22 - protein, 9 aa, Artificial Sequence
  Antibody component sequence
QQSNYWPLT

SEQ ID NO: 23 - protein, 5 aa, Artificial Sequence
  Antibody component sequence
SYWMN

SEQ ID NO: 24 - protein, 6 aa, Artificial Sequence
  Antibody component sequence
GYSFTS

SEQ ID NO: 25 - protein, 10 aa, Artificial Sequence
  Antibody component sequence
GYSFTSYWMN

SEQ ID NO: 26 - protein, 17 aa, Artificial Sequence
  Antibody component sequence
MIHPSDSETRLNQKFKD

SEQ ID NO: 27 - protein, 10 aa, Artificial Sequence
  Antibody component sequence
MIHPSDSETR

SEQ ID NO: 28 - protein, 9 aa, Artificial Sequence
  Antibody component sequence
EMGPYTLDY

SEQ ID NO: 29 - protein, 127 aa, Artificial Sequence
  Antibody component sequence
MSVPTQVLGLLLLWLTDARCDILLTQSPAILSVSPGARVSFSCRASQNIDTSIHWYQQRTNGSPRLLIKYASESISGIPSRFSGSGSGTDFTLSINSVESEDIADYYCQQSNYWPLTFGAGTKLELK

SEQ ID NO: 30 - protein, 141 aa, Artificial Sequence
  Antibody component sequence
MEWSWVFLFFLSVTTGVHSQVQLQQPGAELVRPGASVKLSCKASGYSFTSYWMNWMKQRPGQGLEWIGMIHPSDSETRLNQKFKDKATLTVDKSSSTAYMQLNSPTSEDSAVYYCAREMGPYTLDYWGQGTSVTVSSASTK

SEQ ID NO: 31 - protein, 16 aa, Artificial Sequence
  Antibody component sequence
KSSQSLLNSSNQKNYL

SEQ ID NO: 32 - protein, 7 aa, Artificial Sequence
  Antibody component sequence
FASTRES

SEQ ID NO: 33 - protein, 9 aa, Artificial Sequence
  Antibody component sequence
QQHYSTPPT

SEQ ID NO: 34 - protein, 5 aa, Artificial Sequence
  Antibody component sequence
RYAMS

SEQ ID NO: 35 - protein, 6 aa, Artificial Sequence
  Antibody component sequence
GFTFSR

SEQ ID NO: 36 - protein, 10 aa, Artificial Sequence
  Antibody component sequence
GFTFSRYAMS

SEQ ID NO: 37 - protein, 15 aa, Artificial Sequence
  Antibody component sequence
TIFSGGSYTYYPDSV

SEQ ID NO: 38 - protein, 8 aa, Artificial Sequence
  Antibody component sequence
TIFSGGSY

SEQ ID NO: 39 - protein, 9 aa, Artificial Sequence
  Antibody component sequence
PNWERTFDY

SEQ ID NO: 40 - protein, 133 aa, Artificial Sequence
  Antibody component sequence
MESQTQVLMFLLLWVSGACTDIVMTQSPSSLAMSVGQKVTMSCKSSQSLLNSSNQKNYLAWYQQKPGQSPKLLVYFASTRESGVPDRFMGSGSGTDFTLTISSVQAEDLADYFCQQHYSTPPTFGGGTKLEIK

SEQ ID NO: 41 - protein, 133 aa, Artificial Sequence
  Antibody component sequence
MRVLILLWLFTAFPGLLSDVQLQESGPGLVKPSQSLSLTCTVTGYSITSDYAWNWIRQFPGNKLEWMGFVSYSGTTKYNPSLKSRISITRDTSENQFFLQLNSVTSEDTATYYCARGYGFDYWGQGTTLTVSS

SEQ ID NO: 42 - protein, 38 aa, Artificial Sequence
  Antibody component sequence
EIILTQSPTTMAASPGEKITITCSASSSISSHYLHWYQ

SEQ ID NO: 43 - protein, 40 aa, Artificial Sequence
  Antibody component sequence
QKSGFSPKLLYRTSNLASGVPARESGSGSGTSYSLTIGTM

SEQ ID NO: 44 - protein, 29 aa, Artificial Sequence
  Antibody component sequence
EAEDVATYYCQQGSSLPLTFGAGTKVEIK

SEQ ID NO: 45 - protein, 41 aa, Artificial Sequence
  Antibody component sequence
EIQLQQSGPELVKPGASVKVSCKASGYAFTSQNIYWVKQSH

SEQ ID NO: 46 - protein, 40 aa, Artificial Sequence
  Antibody component sequence
GKSLEWIGYEPYNVVPMYNPKFKGKATLTVDKSSSSAYIH

SEQ ID NO: 47 - protein, 35 aa, Artificial Sequence
  Antibody component sequence
LNSLTSEDSAIYYCARSGSSNFDYWGQGTTLTVSS

SEQ ID NO: 48 - protein, 330 aa, Homo sapiens [sequence omitted]

SEQ ID NO: 49 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 50 - protein, 4 aa, Artificial Sequence
  fusion protein linker sequence
IEGR

SEQ ID NO: 51 - protein, 2 aa, Artificial Sequence
  fusion protein N-terminal component sequence
MD

SEQ ID NO: 52 - protein, 345 aa, Artificial Sequence
  Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 345 aa, Artificial Sequence; identical to SEQ ID NO: 52
  Fusion protein

SEQ ID NO: 54 - protein, 347 aa, Artificial Sequence
  Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - DNA, 25 bp, Artificial Sequence
  Morpholino
cgtcacaggcaggacccactgccca

SEQ ID NO: 56 - protein, 5 aa, Artificial Sequence
  Description of Artificial Sequence Synthetic peptide
DYAMS

SEQ ID NO: 57 - protein, 17 aa, Artificial Sequence
  Description of Artificial Sequence Synthetic peptide
TISDGGTYTYYPDSVKG

SEQ ID NO: 58 - protein, 10 aa, Artificial Sequence
  Description of Artificial Sequence Synthetic peptide
EWGDYDGFDY

SEQ ID NO: 59 - protein, 11 aa, Artificial Sequence
  Description of Artificial Sequence Synthetic peptide
RASQEISGYLS

SEQ ID NO: 60 - protein, 7 aa, Artificial Sequence
  Description of Artificial Sequence Synthetic peptide
AASTLDS

SEQ ID NO: 61 - protein, 9 aa, Artificial Sequence
  Description of Artificial Sequence Synthetic peptide
LQYDSYPYT

SEQ ID NO: 62 - protein, 119 aa, Artificial Sequence
  Description of Artificial Sequence Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 63 - protein, 107 aa, Artificial Sequence
  Description of Artificial Sequence Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 64 - protein, 471 aa, Artificial Sequence
  Description of Artificial Sequence Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 236 aa, Artificial Sequence
  Description of Artificial Sequence Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 451 aa, Artificial Sequence
  mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 121 aa, Artificial Sequence
  mAb
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 68 - protein, 5 aa, Artificial Sequence
  mAb
NYGMN

SEQ ID NO: 69 - protein, 17 aa, Artificial Sequence
  mAb
WINTYTGEPTYTDDFKG

SEQ ID NO: 70 - protein, 12 aa, Artificial Sequence
  mAb
GGFGSSYWYFDV

SEQ ID NO: 71 - protein, 214 aa, Artificial Sequence
  mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 110 aa, Artificial Sequence
  mAb
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTV

SEQ ID NO: 73 - protein, 11 aa, Artificial Sequence
  mAb
KASQDVSIAVA

SEQ ID NO: 74 - protein, 7 aa, Artificial Sequence
  mAb
SASYRYT

SEQ ID NO: 75 - protein, 9 aa, Artificial Sequence
  mAb
QQHYITPLT

SEQ ID NO: 76 - protein, 451 aa, Artificial Sequence
  mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 121 aa, Artificial Sequence
  mAb
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 78 - protein, 5 aa, Artificial Sequence
  mAb
TAGMQ

SEQ ID NO: 79 - protein, 17 aa, Artificial Sequence
  mAb
WINTHSGVPKYAEDFKG

SEQ ID NO: 80 - protein, 12 aa, Artificial Sequence
  mAb
SGFGSSYWYFDV

SEQ ID NO: 81 - protein, 214 aa, Artificial Sequence
  mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 110 aa, Artificial Sequence
  mAb
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTV

SEQ ID NO: 83 - protein, 11 aa, Artificial Sequence
  mAb
KASQDVSTAVA

SEQ ID NO: 84 - protein, 7 aa, Artificial Sequence
  mAb
SASYRYT

SEQ ID NO: 85 - protein, 9 aa, Artificial Sequence
  mAb
QQHYITPLT

SEQ ID NO: 86 - protein, 450 aa, Artificial Sequence
  Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 214 aa, Artificial Sequence
  Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 448 aa, Artificial Sequence
  Antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 214 aa, Artificial Sequence
  Antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 244 aa, Homo sapiens
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR